SEQUENCE LISTING (130 sequences)

SEQ ID NO: 1 - DNA, 1374 bp, Saccharomyces cerevisiae
  CDS (1)..(1374): phosphatidate cytidylyltransferase (YBR029C)
atgtctgacaaccctgagatgaaaccacatggtacgagcaaggagattgtggagtcggttactgacgccacctcaaaggcgattgataaattgcaagaagaactccacaaggacgccagcgaatccgtcacgccggtgaccaaggaaagcactgctgctacaaaggaaagcaggaaatacaactttttcattagaacagtttggacgtttgttatgatcagtggtttcttcatcaccttagcatcgggtcatgcatggtgtatagtgctgattttgggctgccaaattgctacttttaaagagtgtattgccgtaacaagtgcatctggtcgcgaaaagaatttgccattgacaaagacgttgaactggtaccttctcttcaccactatctattacttagatgggaaatcactcttcaagtttttccaagctactttttacgagtatcctgtattgaactttatcgtaacaaatcataagtttatctgctattgtctctatcttatgggatttgttctgttcgtttgtagtttaagaaagggatttttgaaattccagttcggatcattgtgcgttactcatatggttcttcttctggtagtatttcaggctcatttgatcatcaaaaacgtgctcaatgggctattctggttcttattaccatgcgggttggtaattgttaatgatatcttcgcctacctgtgcggcattacattcggtaagactaaactaatagaaatctcgcccaagaaaactttagaaggtttccttggtgcctggtttttcactgctctagctagtattatattaacaaggattctgagcccttatacttacttgacgtgccctgttgaagatctccataccaattttttctctaatttgacatgtgaactaaacccagttttccttccacaagtttacagacttccacccatctttttcgataaagttcaaatcaattcaattacagtaaaaccaatttatttccatgctttaaacttagctacctttgcatcattatttgcgccatttggaggctttttcgcatctggtctaaagagaacttttaaagttaaagactttggccactccatcccaggccatggtggtatcacagacagggttgattgccaattcataatgggttcttttgccaatttgtactatgaaacattcatcagcgaacacagaataacagtagatacagttttatccactattttaatgaacctgaacgacaagcagattatagaattgattgatatcttgattagattcctatctaaaaaaggtataatatcagcgaagaattttgaaaagttggctgacatctttaatgtgaccaagaaatcattgaccaatcactcttga

SEQ ID NO: 2 - protein, 457 aa, Saccharomyces cerevisiae
MSDNPEMKPHGTSKEIVESVTDATSKAIDKLQEELHKDASESVTPVTKESTAATKESRKYNFFIRTVWTFVMISGFFITLASGHAWCIVLILGCQIATFKECIAVTSASGREKNLPLTKTLNWYLLFTTIYYLDGKSLFKFFQATFYEYPVLNFIVTNHKFICYCLYLMGFVLFVCSLRKGFLKFQFGSLCVTHMVLLLVVFQAHLIIKNVLNGLFWFLLPCGLVIVNDIFAYLCGITFGKTKLIEISPKKTLEGFLGAWFFTALASIILTRILSPYTYLTCPVEDLHTNFFSNLTCELNPVFLPQVYRLPPIFFDKVQINSITVKPIYFHALNLATFASLFAPFGGFFASGLKRTFKVKDFGHSIPGHGGITDRVDCQFIMGSFANLYYETFISEHRITVDTVLSTILMNLNDKQIIELIDILIRFLSKKGIISAKNFEKLADIFNVTKKSLTNHS

SEQ ID NO: 3 - DNA, 1104 bp, Brassica napus
  CDS (1)..(1104): phosphatidate cytidylyltransferase (BN04MC30805)
atggaggaggagagcaacgtgaccagcaccagcccgagcaccccggtgcagaggctgaggcacaggaagaggagcagcaccgaggtgctggacggcgacaaggtgaacgccagcccgctgctggtgaacgacaggaacaagtacaagagcttcatggtgaggacctacagcaccctgtggatgatcgccggcttcgtgatggtggtgtacatgggccacctgtacatcaccgccatggtgctggtgatccagatcttcatggccaaggagctgttcaacctgctgaggaaggccccggaggacaagtgcctgccgggcatcaagcagctgaactggcacttcttcttcaccgccatgctgttcgtgtacggcaggatcctgagccagaggctggccaacaccgtgaccgccgaccagttcctgtacaggctggtgagcggcctgatcaagtaccacatggccatctgctacttcctgaacatcatcggcttcatgtggttcatcctgaccctgaagaagaagatgtacaagtaccagttcggccagtacgcctggacccacatgatcctgatcgtggtgttcacccagagcagcttcaccgtggccaacatcttcgagggcatcttctggttcctgctgccggccagcctgatcatcatcaacgacatcttcgcctacatcttcggcttcttcttcggcaggaccccgctgatcaagctgagcccgaagaagacctgggagggcttcatcggcgccagcgtgaccaccatcatcagcgccttcgtgctggccaacgtgctgggcaggttcccgtggctgacctgcccgaggcaggacctgagcaccggctggctgcagtgcgacgccgacccgctgttcaagccggagccgttcaccctgccggcctggatcccgggctggttcccgtggaaggagatggaggtgctgccggtgcagtggcacgccctgtgcctgggcctgttcgccagcatcatcgccccgttcggcggcttcttcgccagcggcttcaagagggccttcaagatcaaggacttcggcgacagcatcccgggccacggcggcatcaccgacaggatggactga

SEQ ID NO: 4 - protein, 367 aa, Brassica napus
MEEESNVTSTSPSTPVQRLRHRKRSSTEVLDGDKVNASPLLVNDRNKYKSFMVRTYSTLWMIAGFVMVVYMGHLYITAMVLVIQIFMAKELFNLLRKAPEDKCLPGIKQLNWHFFFTAMLFVYGRILSQRLANTVTADQFLYRLVSGLIKYHMAICYFLNIIGFMWFILTLKKKMYKYQFGQYAWTHMILIVVFTQSSFTVANIFEGIFWFLLPASLIIINDIFAYIFGFFFGRTPLIKLSPKKTWEGFIGASVTTIISAFVLANVLGRFPWLTCPRQDLSTGWLQCDADPLFKPEPFTLPAWIPGWFPWKEMEVLPVQWHALCLGLFASIIAPFGGFFASGFKRAFKIKDFGDSIPGHGGITDRMD

SEQ ID NO: 5 - DNA, 1278 bp, Zea mays
  CDS (1)..(1278): phosphatidate cytidylyltransferase (ZM06MC30283)
atgcagagggacaccagcagcagcgacgtgagcgccagccacgtgggcagggtgaggaggaggaagcacccgagcgaggccagcaccgacggcaacagggccaacggccagccgctgctggtgaacgaccagaacaagtacaagagcatgctgatcaggacctacagcaccgtgtggatgatcggcggcttcgccttcatcatctacgtgggccacctgtacatctgggccatggtggtggtgatccagatctacatggccagggagctgttcaacctgctgaggaagagcagcgaggagaagcagctgccgggcttcaggctgctgaactggcacttcttcttcaccgccatgctgtacacctacggcaggttcctgagcaggcagctggtgaacaccgtgaccagcgaccacctgctgtacaaggtggtgagcggcctgatcaagtaccagatgttcatctgctacttcctgtacatcgccggcttcgtgtggttcatcctgaccctgaagaagaagacctacaagtaccagttcaagcagtacgcctggacccacatgatcctgctgaccgtgttcgcccagagcgccttcaccgtggccaacatcttcgagggcatcttctggttcctgctgccggccagcctgatcgtgatcaacgacatcttcgcctacctgttcggcttcttcctgggcaggaccccgctgatcaagctgagcccgaagaagacctgggagggcttcatcggcgccagcgtgaccaccatcatcagcgccttcctgctggccaacgtgatgggcaggttccagtggctgacctgcccgaggaaggacctgagcaccggctggctgtactgcgacccgggcccgatgttcaagccggagcactacagcctgggcgagagcgtgccgcactggttcccgtggaaggacctggccatcatgccggtgcagtggcacgccctggccctgggcctgttcgccagcatcatcgccccgttcggcggcttcttcgccagcggcttcaagagggccttcaagatcaaggacttcggcgacagcatcccgggccacggcggcatcaccgacaggatggactgccagatggtgatggccgtgttcgcctacatctaccaccagagcttcatcgccccgcagaacttcagcgtggagatcatcctggaccagatcctgaggaacctgacctacgaggagcagaagtacctgtacgagcagctgggcgagatgttccacgagaggcagctgggccagagctga

SEQ ID NO: 6 - protein, 425 aa, Zea mays
MQRDTSSSDVSASHVGRVRRRKHPSEASTDGNRANGQPLLVNDQNKYKSMLIRTYSTVWMIGGFAFIIYVGHLYIWAMVVVIQIYMARELFNLLRKSSEEKQLPGFRLLNWHFFFTAMLYTYGRFLSRQLVNTVTSDHLLYKVVSGLIKYQMFICYFLYIAGFVWFILTLKKKTYKYQFKQYAWTHMILLTVFAQSAFTVANIFEGIFWFLLPASLIVINDIFAYLFGFFLGRTPLIKLSPKKTWEGFIGASVTTIISAFLLANVMGRFQWLTCPRKDLSTGWLYCDPGPMFKPEHYSLGESVPHWFPWKDLAIMPVQWHALALGLFASIIAPFGGFFASGFKRAFKIKDFGDSIPGHGGITDRMDCQMVMAVFAYIYHQSFIAPQNFSVEIILDQILRNLTYEEQKYLYEQLGEMFHERQLGQS

SEQ ID NO: 7 - DNA, 378 bp, Saccharomyces cerevisiae
  CDS (1)..(378): acyl-carrier protein (YKL192C)
atgtttagatccgtttgccgcatttcttcccgcgtggcaccttctgcgtaccgcactataatgggccgttccgttatgtccaacaccatactcgcacaaagattttattctgcaaacttgagcaaagatcaggtttctcaaagggtcattgatgttatcaaggcgtttgataagaactctcccaacattgccaacaagcaaatctccagcgatacccaatttcacaaggatttggggttggactccttggacactgtcgagctgctcgtagctattgaagaagaatttgatattgaaatccctgacaaagtggctgatgagttgagaagtgttggtgaaacggtcgattatatcgcttccaatcccgacgcaaactaa

SEQ ID NO: 8 - protein, 125 aa, Saccharomyces cerevisiae
MFRSVCRISSRVAPSAYRTIMGRSVMSNTILAQRFYSANLSKDQVSQRVIDVIKAFDKNSPNIANKQISSDTQFHKDLGLDSLDTVELLVAIEEEFDIEIPDKVADELRSVGETVDYIASNPDAN

SEQ ID NO: 9 - DNA, 354 bp, Brassica napus
  CDS (1)..(354): acyl-carrier protein (BN1004MS43616414)
atgcactgcatcaggagcagcatcctgcagcacctgaggctgagggtgccggtgaggagcgtgctgctgctggagaaggagaacgtgctgatcagcaagatgaacttcaccagcggcggcggccaggaccaggtgctgagcaaggtgatcgagctggtgaagaagtacgacaccaccagcgccagcaaggtgaccgagaccgccgacttcaagaaggacctgagcctggacagcctggacagggtggagatcgtgatggccatcgaggaggagttcagcgtggagatcccggacgagaaggccgacaagctgacctgctgcgccgacatcgccagcttcatcgtgagcgagtga

SEQ ID NO: 10 - protein, 117 aa, Brassica napus
MHCIRSSILQHLRLRVPVRSVLLLEKENVLISKMNFTSGGGQDQVLSKVIELVKKYDTTSASKVTETADFKKDLSLDSLDRVEIVMAIEEEFSVEIPDEKADKLTCCADIASFIVSE

SEQ ID NO: 11 - DNA, 384 bp, Glycine max
  CDS (1)..(384): acyl-carrier protein (GM06MC07589)
atgcaaagcataaggaaatctatcttgactcgtgtgaatttgaggagatcaaccgaaagatggtttttgaccagggatgaggttgtgcatatgcaattgagatgttggtgctcttcaacagctgtcagctctgatcaaatactggaccgagtgattgcactggccaagaaatatgataaaattgatggctcaaaggtcactgaaacagctgattttcaaaaagacttgaacctggacagtttggaccgagtggaactcattatggcccttgaagaagaattttctattgaaatccctgatgagaaggctgataagcttgcttgctgtgctgatattgcaaaatacatagcagaggttgatcagaaaaacctggaaaagccctga

SEQ ID NO: 12 - protein, 127 aa, Glycine max
MQSIRKSILTRVNLRRSTERWFLTRDEVVHMQLRCWCSSTAVSSDQILDRVIALAKKYDKIDGSKVTETADFQKDLNLDSLDRVELIMALEEEFSIEIPDEKADKLACCADIAKYIAEVDQKNLEKP

SEQ ID NO: 13 - DNA, 360 bp, Helianthus annuus
  CDS (1)..(360): acyl-carrier protein (HA1004MS66693619)
atggccgccaggaacgccctgctgaagtacctgagggtgaacgtgaccccggccctgcagagcagcagcgcccagaacccgagcgccatcggcggcggcctgatccagctgttcaggaggcacttcagcgaggaggtgaggggcagcttcctggacaagagcgaggtgaccgacagggtggtgacctgcgtgaagaacttccagaaggtggacccgagcaaggtgaccccgaccgcccacttccagaacgacctgggcctggacagcctggacaccgtggaggtggtgatggccctggaggaggagttcggcttcgagatcccggacaacgaggccgacaagatcagcagcatcgagtga

SEQ ID NO: 14 - protein, 119 aa, Helianthus annuus
MAARNALLKYLRVNVTPALQSSSAQNPSAIGGGLIQLFRRHFSEEVRGSFLDKSEVTDRVVTCVKNFQKVDPSKVTPTAHFQNDLGLDSLDTVEVVMALEEEFGFEIPDNEADKISSIE

SEQ ID NO: 15 - DNA, 1191 bp, Saccharomyces cerevisiae
  CDS (1)..(1191): acyltransferase (YDR018C)
atgaagcattcccaaaaataccgtaggtatggaatttatgaaaagactggtaatccctttataaaagggttgcaaaggctgcttatcgcttgcttgttcatttcaggctcgctgagtattgtcgtttttcagatctgtctacaggtgcttctcccttggagcaagattagatttcaaaatggtataaatcaaagtaagaaggcttttatcgttttattatgcatgatcttgaacatggtggctccctcttctttgaatgtcacttttgaaacatcgcggccattgaagaactcttctaacgccaagccatgctttagatttaaagacagggctataataattgcaaatcatcaaatgtatgcagactggatttatctctggtggctttcctttgtttcaaatttgggtggtaacgtttatatcatcctgaagaaagctctgcagtacataccattactgggatttggcatgcgaaattttaagtttatatttttaagtaggaactggcaaaaggatgagaaagctttaacaaatagtttggtttctatggacttaaacgcgaggtgcaaggggccccttacaaattataagagttgttattccaagacaaatgaatccattgccgcttataatttaatcatgttccctgagggtacaaatctaagcctcaagacaagagaaaaaagcgaggcattctgtcaaagagcacatttggaccatgtccaattaagacatttgttattaccgcactctaaaggcttgaagtttgcagtagaaaaactagctcctagtttagatgctatctacgatgtcactattggatattctcccgccttgagaacggaatacgtcggcaccaaattcaccttgaagaaaatattcttaatgggtgtctatccggagaaagtagatttttatattagggaatttagagttaatgagatccctttgcaagatgacgaagtttttttcaattggttactgggcgtgtggaaagaaaaagatcaactgctagaagactactacaacacaggccaatttaaaagtaatgctaaaaatgacaaccaatccatcgttgttacgacacaaacgactggatttcagcacgaaacattgacaccccgtatcctttcatattacgggttcttcgcttttcttattcttgtatttgtgatgaaaaaaaatcattga

SEQ ID NO: 16 - protein, 396 aa, Saccharomyces cerevisiae
MKHSQKYRRYGIYEKTGNPFIKGLQRLLIACLFISGSLSIVVFQICLQVLLPWSKIRFQNGINQSKKAFIVLLCMILNMVAPSSLNVTFETSRPLKNSSNAKPCFRFKDRAIIIANHQMYADWIYLWWLSFVSNLGGNVYIILKKALQYIPLLGFGMRNFKFIFLSRNWQKDEKALTNSLVSMDLNARCKGPLTNYKSCYSKTNESIAAYNLIMFPEGTNLSLKTREKSEAFCQRAHLDHVQLRHLLLPHSKGLKFAVEKLAPSLDAIYDVTIGYSPALRTEYVGTKFTLKKIFLMGVYPEKVDFYIREFRVNEIPLQDDEVFFNWLLGVWKEKDQLLEDYYNTGQFKSNAKNDNQSIVVTTQTTGFQHETLTPRILSYYGFFAFLILVFVMKKNH

SEQ ID NO: 17 - DNA, 1155 bp, Glycine max
  CDS (1)..(1155): acyltransferase (GM06MC27072)
atgaccgccgtggtggtggtgccgctgggcctgctgttcttcgccagcggcctgatcgtgaacctgatccaggccatctgctacgtggtggtgaggccggtgagcaagaacctgtacaggaggatgaacagggtggtggccgagctgctgtggctggagctggtgtggatcatcgactggtgggccggcgtgaaggtgcaggtgttcaccgacccggagaccttccacagcatgggcaaggagcacgccctggtgatcagcaaccacaggagcgacatcgactggctggtgggctgggtgctggcccagaggagcggctgcctgggcagcaccctggccgtgatgaagaagagcagcaagttcctgccggtgatcggctggagcatgtggttcagcgagtacctgttcctggagaggagctgggccaaggacgagaggaccctgaagagcggcctgcagcagctgagggacttcccgctgccgttctggctggccctgttcgtggagggcaccaggttcacccaggccaagctgctggccgcccaggagtacgccgccagcgccggcctgccggtgccgaggaacgtgctgatcccgaggaccaagggcttcgtgagcgccgtgaaccacatgaggagcttcgtgccggccatctacgacgtgaccgtggccatcccgaagagcagcccggccccgaccatgctgaggctgttcaggggcaagagcagcctggtgcacgtgcacatcaagaggcacgccatgaaggacctgccggaggaggacgaggccgtggcccagtggtgcagggacgtgttcgtggccaaggacgccctgctggacaagcacatcgccgaggacaccttcagcgaccaggagctgcaggacaccggcaggccgatcaagagcctgctggtggtgatcagctgggtgtgcctggtggtggccggcagcgtgaagttcctgcagaggagcagcctgctgagcagctggaagggcgtggccttcagcgccttcggcctggccgtggtgaccgccctgatgcagatcctgatccagttcagccagagcgagaggagcaacccggccaagatcgtgccggccaagagcaagaacaagggcggccagctggaggccaggaacgacaagcagcagtga

SEQ ID NO: 18 - protein, 384 aa, Glycine max
MTAVVVVPLGLLFFASGLIVNLIQAICYVVVRPVSKNLYRRMNRVVAELLWLELVWIIDWWAGVKVQVFTDPETFHSMGKEHALVISNHRSDIDWLVGWVLAQRSGCLGSTLAVMKKSSKFLPVIGWSMWFSEYLFLERSWAKDERTLKSGLQQLRDFPLPFWLALFVEGTRFTQAKLLAAQEYAASAGLPVPRNVLIPRTKGFVSAVNHMRSFVPAIYDVTVAIPKSSPAPTMLRLFRGKSSLVHVHIKRHAMKDLPEEDEAVAQWCRDVFVAKDALLDKHIAEDTFSDQELQDTGRPIKSLLVVISWVCLVVAGSVKFLQRSSLLSSWKGVAFSAFGLAVVTALMQILIQFSQSERSNPAKIVPAKSKNKGGQLEARNDKQQ

SEQ ID NO: 19 - DNA, 999 bp, Zea mays
  CDS (1)..(999): acyltransferase (ZM06MC04863)
atggagtgcagcaacagcaccagcagccagggccaccacgtgggcaagtggaaccaggtgcagatcatcgacccgagcatcgtggagatgggcccgagccacctgccgctgagcttcatgaggaggtgcaggggcgtgctgtgcctggtgatcatgatcctgaccgccttcatgatgatggtgtacctgagcccggtgaccaccttcctggtgaggctgttcagcgtgcactacagcaggaagagcacctgcttcctgttcggcatgtggctggccatgtggccgttcctgttcgagaagatcaacaagaccaggttcgtgttcagcggcgagagcgtgccggccaaggagagggtgctgctgttcgccaaccacaggaccgaggtggactggatgtacctgtgggacttcgccctgaggaagggcaggctgcagtgcatcaagtacatcctgaagaagagcctgatgaagctgccggtgttcaactgggccttccacatcatcgagttcatcccggtggagaggaagtgggagatcgacgaggccatcatcaggagcaggctgagcgagttcaagaacccgaaggacccgctgtggctggccgtgttcccggagggcaccgactacaccgagaagaagtgcatcaagagccaggagtacgccgccgagcacggcctgccggtgctgaagaacgtgctgctgccgaagaccaagggcttcaactgctgcctgcaggtgctgaggagcaccatcgacgccgtgtacgacatcaccatcgcctacaagcacaggccgccgaccttcctggacaacgtgtacggcgtgggcctgaggaagagcagcagcaccagcgccgccagcaggagcccgacctacaggaggccgaagaccggctggctggccggctggtggagcggcagcggcagcaggaccagctgctgcccggccagcccgaggtgggccaccagcccgaccaagggcccgccgaggggcacctga

SEQ ID NO: 20 - protein, 332 aa, Zea mays
MECSNSTSSQGHHVGKWNQVQIIDPSIVEMGPSHLPLSFMRRCRGVLCLVIMILTAFMMMVYLSPVTTFLVRLFSVHYSRKSTCFLFGMWLAMWPFLFEKINKTRFVFSGESVPAKERVLLFANHRTEVDWMYLWDFALRKGRLQCIKYILKKSLMKLPVFNWAFHIIEFIPVERKWEIDEAIIRSRLSEFKNPKDPLWLAVFPEGTDYTEKKCIKSQEYAAEHGLPVLKNVLLPKTKGFNCCLQVLRSTIDAVYDITIAYKHRPPTFLDNVYGVGLRKSSSTSAASRSPTYRRPKTGWLAGWWSGSGSRTSCCPASPRWATSPTKGPPRGT

SEQ ID NO: 21 - DNA, 2142 bp, Escherichia coli
  CDS (1)..(2142): bifunctional anaerobic fatty acid oxidation complex protein (B2341)
atggaaatgacatcagcgtttacccttaatgttcgtctggacaacattgccgttatcaccatcgacgtaccgggtgagaaaatgaataccctgaaggcggagtttgcctcgcaggtgcgcgccattattaagcaactccgtgaaaacaaagagttgcgaggcgtggtgtttgtctccgctaaaccggacaacttcattgctggcgcagacatcaacatgatcggcaactgcaaaacggcgcaagaagcggaagctctggcgcggcagggccaacagttgatggcggagattcatgctttgcccattcaggttatcgcggctattcatggcgcttgcctgggtggtgggctggagttggcgctggcgtgccacggtcgcgtttgtactgacgatcctaaaacggtgctcggtttgcctgaagtacaacttggattgttacccggttcaggcggcacccagcgtttaccgcgtctgataggcgtcagcacagcattagagatgatcctcaccggaaaacaacttcgggcgaaacaggcattaaagctggggctggtggatgacgttgttccgcactccattctgctggaagccgctgttgagctggcaaagaaggagcgcccatcttcccgccctctacctgtacgcgagcgtattctggcggggccgttaggtcgtgcgctgctgttcaaaatggtcggcaagaaaacagaacacaaaactcaaggcaattatccggcgacagaacgcatcctggaggttgttgaaacgggattagcgcagggcaccagcagcggttatgacgccgaagctcgggcgtttggcgaactggcgatgacgccacaatcgcaggcgctgcgtagtatcttttttgccagtacggacgtgaagaaagatcccggcagtgatgcgccgcctgcgccattaaacagcgtggggattttaggtggtggcttgatgggcggcggtattgcttatgtcactgcttgtaaagcggggattccggtcagaattaaagatatcaacccgcagggcataaatcatgcgctgaagtacagttgggatcagctggagggcaaagttcgccgtcgtcatctcaaagccagcgaacgtgacaaacagctggcattaatctccggaacgacggactatcgcggctttgcccatcgcgatctgattattgaagcggtgtttgaaaatctcttgaaacaacagatggtggcggaagttgagcaaaattgcgccgctcataccatctttgcttcgaatacgtcatctttaccgattggtgatatcgccgctcacgccacgcgacctgagcaagttatcggcctgcatttcttcagtccggtggaaaaaatgccgctggtggagattattcctcatgcggggacatcggcgcaaaccatcgctaccacagtaaaactggcgaaaaaacagggtaaaacgccaattgtcgtgcgtgacaaagccggtttttacgtcaatcgcatcttagcgccttacattaatgaagctatccgcatgttgacccaaggtgaacgggtagagcacattgatgccgcgctagtgaaatttggttttccggtaggcccaatccaacttttggatgaggtaggaatcgacaccgggactaaaattattcctgtactggaagccgcttatggagaacgttttagcgcgcctgcaaatgttgtttcttcaattttgaacgacgatcgcaaaggcagaaaaaatggccggggtttctatctttatggtcagaaagggcgtaaaagcaaaaaacaggtcgatcccgccatttacccgctgattggcacacaagggcaggggcgaatctccgcaccgcaggttgctgaacggtgtgtgatgttgatgctgaatgaagcagtacgttgtgttgatgagcaggttatccgtagcgtgcgtgacggggatattggcgcggtatttggcattggttttccgccatttctcggtggaccgttccgctatatcgattctctcggcgcgggcgaagtggttgcaataatgcaacgacttgccacgcagtatggttcccgttttaccccttgcgagcgtttggtcgagatgggcgcgcgtggggaaagtttttggaaaacaactgcaactgacctgcaataa

SEQ ID NO: 22 - protein, 713 aa, Escherichia coli
MEMTSAFTLNVRLDNIAVITIDVPGEKMNTLKAEFASQVRAIIKQLRENKELRGVVFVSAKPDNFIAGADINMIGNCKTAQEAEALARQGQQLMAEIHALPIQVIAAIHGACLGGGLELALACHGRVCTDDPKTVLGLPEVQLGLLPGSGGTQRLPRLIGVSTALEMILTGKQLRAKQALKLGLVDDVVPHSILLEAAVELAKKERPSSRPLPVRERILAGPLGRALLFKMVGKKTEHKTQGNYPATERILEVVETGLAQGTSSGYDAEARAFGELAMTPQSQALRSIFFASTDVKKDPGSDAPPAPLNSVGILGGGLMGGGIAYVTACKAGIPVRIKDINPQGINHALKYSWDQLEGKVRRRHLKASERDKQLALISGTTDYRGFAHRDLIIEAVFENLLKQQMVAEVEQNCAAHTIFASNTSSLPIGDIAAHATRPEQVIGLHFFSPVEKMPLVEIIPHAGTSAQTIATTVKLAKKQGKTPIVVRDKAGFYVNRILAPYINEAIRMLTQGERVEHIDAALVKFGFPVGPIQLLDEVGIDTGTKIIPVLEAAYGERFSAPANVVSSILNDDRKGRKNGRGFYLYGQKGRKSKKQVDPAIYPLIGTQGQGRISAPQVAERCVMLMLNEAVRCVDEQVIRSVRDGDIGAVFGIGFPPFLGGPFRYIDSLGAGEVVAIMQRLATQYGSRFTPCERLVEMGARGESFWKTTATDLQ

SEQ ID NO: 23 - DNA, 2172 bp, Zea mays
  CDS (1)..(2172): bifunctional anaerobic fatty acid oxidation complex protein (ZM06MC04303)
atggccgccaaggtgaggaccgagctggacgtgggcgccgacggcgtggccgtgatcaccatctacaacccgccggtgaacagcctgagcatcgacgtgctgcacagcctgaaggagagctacgaggaggccctgaggaggaacgacgtgaaggccatcgtggtgaccggcaagggcggcaagttcagcggcggcttcgacatcagcagcttcggcggcgtgcagggcggccagaccatgcagccgaaggtgggctacatcgccatcgacatcctgaccgacaccgtggaggccgccaccaagccgagcgtggccgccatcgacggcctggccctgggcggcggcctggaggtggccatggcctgccacgccaggatcgccaccccgaccgcccagctgggcctgccggagctgcagctgggcatcatcccgggcttcggcggcacccagaggctgccgaggctggtgggcctgaccaagagcctggagatgatgctgctgagcaagccgatcaagggcggcgaggcccaccagctgggcctggtggacgccctggtgagcccgaacgacctggtgaacaccgccaggcagtgggccctggacatctacgagtgcaggaggccgtggatcaagagcctgtacaagaccgacaagctggagccgctgggcgaggccagggagatcctgaagttcgccagggcccaggcccagaagcaggccgccaacctgcaccacccgctggtgtgcatcgacgtgatcgaggagggcatcgtggccggcccgagggccggcctgtggaaggaggccaccagcttccaggagctgctgttcagcgacacctgcaagagcctggtgcacgtgttcttcagccagagggccaccagcaagatcccgggcgccaccgacctgggcctgatgccgaggaagatcaccaaggtggccatcctgggcggcggcctgatgggcagcggcatcgccaccgccatggtgctgagcaactacccggtgctgctgaaggaggtgaacgagaagttcctgaccgccggcatcaacaggatccaggccaacctgcagagcagggtgaagaagggcaagatgaccgaggagaggtacgagaaggccatgagcctggtgaccggcgtgctggactacgagaggttcaaggacgtggacctggtgatcgaggccgtgatcgagaacgtgaagctgaagcagcagatcttcagcgacctggagaagtactgcccgagccactgcatcctggccaccaacaccagcaccatcgacctgaacctgatcggcgagaagaccaaggcccaggacaggatcgccggcgcccacttcttcagcccggcccacgtgatgccgctgctggagatcgtgaggacccagcacaccagcccgcaggtggtggtggacctgctggacgtgggcaagaagatcaagaagaccccgatcgtggtgggcaactgcaccggcttcgccgtgaacaggatgttcttcccgtacacccagagcgccctgttctacgtggacctgggcatggacgtgtacaagatcgacagggcctgcaccaagttcggcatgccgatgggcccgttcaggctggccgacctggtgggcttcggcgtggccgtggccaccggcatgcagtacctggagaacttcccggagagggtgtacaagagcatgctgctgccgctgatgatggagggcaacagggccggcgaggccacccagaagggcttctacaagtacgagggcaagaggaaggccaccccggacccggagatcatgaagtacatcgagaagagcaggagcatggccggcgtgaccccggacccggagctgatgaagctgagcgagaaggacatcgtggagatggtgttcttcccggtgatcaacgaggcctgcagggtgctggacgagggcatcgccgtgaaggccagcgacctggacatcgccagcatcttcggcatgggcttcccgccgtacaggggcggcgtgatgcactgggccgacagcatcggcgccaagtacatccacggcaagctggaggagtggaccaagaggtacggcggcttcttcaagccgtgcagctacctggccgagagggccgccaagggcatcccgctgagcgccccgaccaagaaggtgcaggccaggctgtga

SEQ ID NO: 24 - protein, 723 aa, Zea mays
MAAKVRTELDVGADGVAVITIYNPPVNSLSIDVLHSLKESYEEALRRNDVKAIVVTGKGGKFSGGFDISSFGGVQGGQTMQPKVGYIAIDILTDTVEAATKPSVAAIDGLALGGGLEVAMACHARIATPTAQLGLPELQLGIIPGFGGTQRLPRLVGLTKSLEMMLLSKPIKGGEAHQLGLVDALVSPNDLVNTARQWALDIYECRRPWIKSLYKTDKLEPLGEAREILKFARAQAQKQAANLHHPLVCIDVIEEGIVAGPRAGLWKEATSFQELLFSDTCKSLVHVFFSQRATSKIPGATDLGLMPRKITKVAILGGGLMGSGIATAMVLSNYPVLLKEVNEKFLTAGINRIQANLQSRVKKGKMTEERYEKAMSLVTGVLDYERFKDVDLVIEAVIENVKLKQQIFSDLEKYCPSHCILATNTSTIDLNLIGEKTKAQDRIAGAHFFSPAHVMPLLEIVRTQHTSPQVVVDLLDVGKKIKKTPIVVGNCTGFAVNRMFFPYTQSALFYVDLGMDVYKIDRACTKFGMPMGPFRLADLVGFGVAVATGMQYLENFPERVYKSMLLPLMMEGNRAGEATQKGFYKYEGKRKATPDPEIMKYIEKSRSMAGVTPDPELMKLSEKDIVEMVFFPVINEACRVLDEGIAVKASDLDIASIFGMGFPPYRGGVMHWADSIGAKYIHGKLEEWTKRYGGFFKPCSYLAERAAKGIPLSAPTKKVQARL

SEQ ID NO: 25 - DNA, 2184 bp, Zea mays
  CDS (1)..(2184): bifunctional anaerobic fatty acid oxidation complex protein (ZM06MC15742)
atggccgccggcagcatcagggtgaccatggaggtgggcgccgacggcgtggccctgatcaccatcgccaacccgccggtgaacgccctgcacccgatcatcatcgccggcctgaaggacaagtacgccgaggccctgaggagggacgacgtgaaggccatcgtgctgaccggcgccggcggcaagttctgcggcggcttcgacatcaacgtgttcaccaaggtgcaccagaccggcgacgtgagcctgatgccggacgtgagcgtggagctggtgagcaacatgatggaggagggcaagaagccgagcgtggccgccatccagggcctggccctgggcggcggcctggagctgaccatgggctgccacgccaggatcagcaccccggaggcccagctgggcctgccggagctgaccctgggcatcatcccgggcttcggcggcacccagaggctgccgaggctggtgggcctgccgaaggccatcgagatgatgctgcagagcaagttcatcaccgccaaggagggcaaggagaggggcctgatcgacgccctgtgcagcccggacgagctgatcaagaccagcaggctgtgggccctggagatcgccaactgcaggaagccgtggatgaggagcctgggcaggaccgacaggctgggcccgctgagcgaggccagggccgtgctgaacgccgccaggcagcaggccatgaagatcgccccgaacatgccgcagaaccaggcctgcctggacgtgatggaggagggcatcctgtgcggcggccaggccggcgtgctgaaggaggccatggtgttcaaggagctggtgatcgccccgaccagcaaggccctggtgcacgtgttcttcgcccagaggagcaccaccaaggtgccgggcgtgaccgacgtgcagctgaagccgaggccgatcaggaaggtggccgtgatcggcggcggcctgatgggcagcggcatcgccaccagcctgctggtgagcaacatcagcgtggtgctgaaggaggtgaacccgcagttcctgcagaggggcgagaagaccatcgccggcaacctggagggcctggtgaagaggggcagcccgaccaaggacaggatgcacaaggccatggccctgctgaagggcgccctggactacagcgacttcaaggacgtggacatggtgatcgaggccgtgatcgagaagatcccgctgaagcagagcatcttcgccgacatcgagaagatctgcccgaagcactgcatcctggccaccaacaccagcaccatcgacctgaacgtggtgggcaagaagaccaacagccaggacaggatcatcggcgcccacttcttcagcccggcccacatcatgccgctgctggagatcgtgaggaccgagaagaccagcccgcaggccatcctggacctgatcaccatcggcaagatcatcaagaaggtgccgatcgtggtgggcaactgcaccggcttcgccgtgaacctgaccttcttcccgtacacccagggcagccacctgctggtgagcctgggcatcgacgtgttcaggatcgacagggtgatcagcaccttcggcatgccgatgggcccgttccagctgcaggacgtggccggctacggcgtggccctggccgtgaaggacatctacgccgacgccttcggcgagaggaacctggacagcgacctggtggacctgatggtgaaggacggcaggcagggcaaggtgaacggcaagggctactacatctacgagaagggcggcaagccgaagccggacccgagcgtgaagcacgtgatcgaggagtacaggaagcacgccaacaccatgccgggcggcaagccggtgaccctgaccgaccaggacatcctggagatgatcttcttcccggtggtgaacgaggcctgcagggtgatggacgagaacgtggtgatcagggccagcgacctggacatcgccagcgtgctgggcatgggcttcccgaagtacaggggcggcctggtgttctgggccgacaccgtgggcgccccgtacatccacagcaagctgagcaagtgggccgagatctacggcccgttcttcaagccgagcagctacctggagcagagggccaagagcggcgtgccgctgagcgccccgggcgccagccagcagggcagcgccaggagcaggatgtga

SEQ ID NO: 26 - protein, 727 aa, Zea mays
MAAGSIRVTMEVGADGVALITIANPPVNALHPIIIAGLKDKYAEALRRDDVKAIVLTGAGGKFCGGFDINVFTKVHQTGDVSLMPDVSVELVSNMMEEGKKPSVAAIQGLALGGGLELTMGCHARISTPEAQLGLPELTLGIIPGFGGTQRLPRLVGLPKAIEMMLQSKFITAKEGKERGLIDALCSPDELIKTSRLWALEIANCRKPWMRSLGRTDRLGPLSEARAVLNAARQQAMKIAPNMPQNQACLDVMEEGILCGGQAGVLKEAMVFKELVIAPTSKALVHVFFAQRSTTKVPGVTDVQLKPRPIRKVAVIGGGLMGSGIATSLLVSNISVVLKEVNPQFLQRGEKTIAGNLEGLVKRGSPTKDRMHKAMALLKGALDYSDFKDVDMVIEAVIEKIPLKQSIFADIEKICPKHCILATNTSTIDLNVVGKKTNSQDRIIGAHFFSPAHIMPLLEIVRTEKTSPQAILDLITIGKIIKKVPIVVGNCTGFAVNLTFFPYTQGSHLLVSLGIDVFRIDRVISTFGMPMGPFQLQDVAGYGVALAVKDIYADAFGERNLDSDLVDLMVKDGRQGKVNGKGYYIYEKGGKPKPDPSVKHVIEEYRKHANTMPGGKPVTLTDQDILEMIFFPVVNEACRVMDENVVIRASDLDIASVLGMGFPKYRGGLVFWADTVGAPYIHSKLSKWAEIYGPFFKPSSYLEQRAKSGVPLSAPGASQQGSARSRM

SEQ ID NO: 27 - DNA, 861 bp, Escherichia coli
  CDS (1)..(861): Acyl-CoA thioesterase (B0452)
atgagtcaggcgctaaaaaatttactgacattgttaaatctggaaaaaattgaggaaggactctttcgcggccagagtgaagatttaggtttacgccaggtgtttggcggccaggtcgtgggtcaggccttgtatgctgcaaaagagaccgtccctgaagagcggctggtacattcgtttcacagctactttcttcgccctggcgatagtaagaagccgattatttatgatgtcgaaacgctgcgtgacggtaacagcttcagcgcccgccgggttgctgctattcaaaacggcaaaccgattttttatatgactgcctctttccaggcaccagaagcgggtttcgaacatcaaaaaacaatgccgtccgcgccagcgcctgatggcctcccttcggaaacgcaaatcgcccaatcgctggcgcacctgctgccgccagtgctgaaagataaattcatctgcgatcgtccgctggaagtccgtccggtggagtttcataacccactgaaaggtcacgtcgcagaaccacatcgtcaggtgtggatccgcgcaaatggtagcgtgccggatgacctgcgcgttcatcagtatctgctcggttacgcttctgatcttaacttcctgccggtagctctacagccgcacggcatcggttttctcgaaccggggattcagattgccaccattgaccattccatgtggttccatcgcccgtttaatttgaatgaatggctgctgtatagcgtggagagcacctcggcgtccagcgcacgtggctttgtgcgcggtgagttttatacccaagacggcgtactggttgcctcgaccgttcaggaaggggtgatgcgtaatcacaattaa

SEQ ID NO: 28 - protein, 286 aa, Escherichia coli
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 29 - DNA, 747 bp, Brassica napus
  CDS (1)..(747): Acyl-CoA thioesterase (BN42634969)
atgaagttagtccatagtttacacgcctatttccttctttctggagatactaatattcccatcatatatgaagttagccgcttacgcgatggcaacaattttgccacccgaagagtagatgcaagacagaaaggaaaaaccatattcaccttgttcgcatcatttcagagagagcaacaaggttttgatcaccaggagtcgaacatgcctcatatgctacctcctgaaacgcttgtatcaagggatgaaatgcttcaacggcgtatgactgaccatctgatacctaggtattaccgaaataaagttgcaacccaatatactgccccactgcctatagatattcgattttgtgagccaaattactctacagaagagagaaagtctccttcaagattgaactattggtttaaggcaaggggaaaactttctgatgaccaagctttacaccgatgtgtggttgcatttgcttcagatctgatatttgcatgtatcggtttaaaccctcaccgtagaaagggcatgagtgcagctgctcttagcctagaccactcgttgtggttccaccgacctctaagagctgatgactggcttctctttgtgatggtgaatcccacatcattccagagtcgtggtctgaccactggagaaatgttcaacagaaaaggagagctagtggtatcattgacgcaagaagcattgctaaaagaagcggtgacgattaagcccatcttcggcgccaagctctaa

SEQ ID NO: 30 - protein, 248 aa, Brassica napus
MKLVHSLHAYFLLSGDTNIPIIYEVSRLRDGNNFATRRVDARQKGKTIFTLFASFQREQQGFDHQESNMPHMLPPETLVSRDEMLQRRMTDHLIPRYYRNKVATQYTAPLPIDIRFCEPNYSTEERKSPSRLNYWFKARGKLSDDQALHRCVVAFASDLIFACIGLNPHRRKGMSAAALSLDHSLWFHRPLRADDWLLFVMVNPTSFQSRGLTTGEMFNRKGELVVSLTQEALLKEAVTIKPIFGAKL

SEQ ID NO: 31 - DNA, 639 bp, Brassica napus
  CDS (1)..(639): Acyl-CoA thioesterase (BNP5302_30)
atgtctcatcattgttctttgcttgacaccaaccctgtctccgatccagctaaagatcttgagaccccctgtcttgtggaacgcattttgtctctccatccattatatttgactctttttcgaggtttcactatacccaatgctataacctttggcaaggtttttggacgacaattagttggacaggcacttgccgcaccgacaaacactgatgaatctaccaagattgttcatagtttacactcctatttcctactcgttggagatataactattcccatcctatacgaagttaaccacttacgtgatggcaacaactttgccacccgaagagttgatgctagacagaaaggcaaaaccattttcatcttgtttgcttcatttcagagagatcaacaaggtttcgatcaccaagagtcgaacatgcctcttatgtcacctcctgaaacgcttgtaacaagagaggagatgattgaacggcgtatgactgaccctctgctacctagggattaccgaaacaaaattgcagctgaaaaaatcctcacatggcctatagacattcgattttgtgagccaagttattatacagaacatacaaagtctcctccaagattgaactattggtttaaggcaaggtga

SEQ ID NO: 32 - protein, 212 aa, Brassica napus
MSHHCSLLDTNPVSDPAKDLETPCLVERILSLHPLYLTLFRGFTIPNAITFGKVFGRQLVGQALAAPTNTDESTKIVHSLHSYFLLVGDITIPILYEVNHLRDGNNFATRRVDARQKGKTIFILFASFQRDQQGFDHQESNMPLMSPPETLVTREEMIERRMTDPLLPRDYRNKIAAEKILTWPIDIRFCEPSYYTEHTKSPPRLNYWFKAR

SEQ ID NO: 33 - DNA, 594 bp, Glycine max
  CDS (1)..(594): Acyl-CoA thioesterase (GMsae90f11)
atggcttcatttcaaaaagaagaatcagggatggtccaccaagaggtagctataccatctgtccctgctccagataagcttctgccgatggaagagctacgggagagacgtcttactgaccctcgtttaccaataacctatcggaacaaagtagctacatctcaattcatcccatggcccatagagatacgattatgtgaatatgaaactgcaacaaatatgacaaaatctcctcccagtttgagatactggtttagagccaagggaaaactttcagatgatcaagccttgcataggtgtgtggtagcatatacatcagatctaatcttccttcaagtgagtttgaaccccaaccgtaggaagggaaggaaggctcgtgctgtgagtctggaccactccatgtggtttcacagacctttaagagctgatgattggatactatttgtgatctttagtcctactgccaataatgcccgcggctatgtcactggccaaatgttcaatcagaagggagagcatcttgtgtctgtggttcaagaaggtgtaatgagggaagttatttctgctaagtcagccatcaaatctaatctatga

SEQ ID NO: 34 - protein, 197 aa, Glycine max
MASFQKEESGMVHQEVAIPSVPAPDKLLPMEELRERRLTDPRLPITYRNKVATSQFIPWPIEIRLCEYETATNMTKSPPSLRYWFRAKGKLSDDQALHRCVVAYTSDLIFLQVSLNPNRRKGRKARAVSLDHSMWFHRPLRADDWILFVIFSPTANNARGYVTGQMFNQKGEHLVSVVQEGVMREVISAKSAIKSNL

SEQ ID NO: 35 - DNA, 888 bp, Saccharomyces cerevisiae
  CDS (1)..(888): 2,4-dienoyl-CoA reductase (YNL202W)
atggatactatgaatacagcaaacactttggacggcaaattcgttactgagggttcttggagacctgatttatttaagggtaaagtggcatttgtcactggtggagctggcacgatatgtcgggtacagacagaagccttggttcttcttggctgtaaggcagctattgtcggcagggaccaagaaagaacagaacaggcagcaaaaggtatttcgcagttagcaaaggacaaagatgcagtcttggctattgcaaacgtcgatgttcgcaattttgaacaagtggagaatgcggttaaaaagaccgtagagaagttcggtaagatcgatttcgtcattgccggtgctgctggaaattttgtgtgcgattttgcgaacctctctccaaacgccttcaaatctgttgttgacatagatttactaggaagttttaataccgccaaggcatgcttgaaggagttaaaaaagtcaaaaggttccatcctttttgtctctgccactttccattattatggtgtgccgtttcaaggacatgtgggcgctgctaaagcgggaatagacgcattggcaaagaatttagcggttgagttggggcctctgggtatacgttcgaattgcattgctccaggtgctatcgataatacggaaggcttgaagagattagccggcaaaaaatataaggaaaaggccttggcaaagataccattacaaaggcttggatctacacgggatatagccgaatctactgtttatattttttcacccgccgcatcgtatgtcaccggtgccgtcttagttgttgacggtggtatgtggcatttagggacctattttggacatgagttgtatccagaagccttaataaagagtatgacatctaaattataa

SEQ ID NO: 36 - protein, 295 aa, Saccharomyces cerevisiae
MDTMNTANTLDGKFVTEGSWRPDLFKGKVAFVTGGAGTICRVQTEALVLLGCKAAIVGRDQERTEQAAKGISQLAKDKDAVLAIANVDVRNFEQVENAVKKTVEKFGKIDFVIAGAAGNFVCDFANLSPNAFKSVVDIDLLGSFNTAKACLKELKKSKGSILFVSATFHYYGVPFQGHVGAAKAGIDALAKNLAVELGPLGIRSNCIAPGAIDNTEGLKRLAGKKYKEKALAKIPLQRLGSTRDIAESTVYIFSPAASYVTGAVLVVDGGMWHLGTYFGHELYPEALIKSMTSKL

SEQ ID NO: 37 - DNA, 795 bp, Helianthus annuus
  CDS (1)..(795): 2,4-dienoyl-CoA reductase (HA66688442)
atggcgcttccatcgctgtcatgggacgtcgcaaaaccgtcgttgactctgccgactcgtccctccagtcacttggtattccggctgtcggcttttgcgggtgatgttcgcaagcaggaagatgcaaagagagtgatagaatcaaccgttaaacactttggaaagctcgatattcttgtgaactctgcagccgggaattttctggtttcacccgaagatctatctccaaatggctttaaaacagtgatggacatcgactcggttggtacgtttaccatgtgccatgaagcaatcaattatctaaagaaaggaggacccggaagaagctcagatgacagtggtggaataatactgaatatcagtgccaccctgcactacactgctacttggtaccaaattcatgtagctgctgctaaggcagcagtggatgctattacaagaaatctggcactggaatggggaacggattatgatatacgcgtcaatgggattgcacctggaccgataggcgacactgctggcgtgaggaaacttgggccagctgagataaaaagcgggaccagggagataatgccgctttttaaacttggagaaaagtgggatattgcggttgcagccgtgtatcttgcatcagatgcgggtaaatttgtgaatgggacgacgcttgttgttgatggaggacagtggttaagtggtccgcgtcatatgcccaaggaggaagtgaaaatgctttctagggtagtagaaaaacgcgctcgtgttgcacctactggggtaccttctagcaaactctag

SEQ ID NO: 38 - protein, 264 aa, Helianthus annuus
MALPSLSWDVAKPSLTLPTRPSSHLVFRLSAFAGDVRKQEDAKRVIESTVKHFGKLDILVNSAAGNFLVSPEDLSPNGFKTVMDIDSVGTFTMCHEAINYLKKGGPGRSSDDSGGIILNISATLHYTATWYQIHVAAAKAAVDAITRNLALEWGTDYDIRVNGIAPGPIGDTAGVRKLGPAEIKSGTREIMPLFKLGEKWDIAVAAVYLASDAGKFVNGTTLVVDGGQWLSGPRHMPKEEVKMLSRVVEKRARVAPTGVPSSKL

SEQ ID NO: 39 - DNA, 1647 bp, Saccharomyces cerevisiae
  CDS (1)..(1647): sterol esterase (YKL140W)
atgtacttcccctttttaggcagattatcgataacagattacattatagtcgtcttggtatacatagaaagcattatttcatcagtccttaaacttataccacaaccaatgattaaccttttcgaatggctaataaatttctcaacgtcctccgatgacaataccatcgaagaaaagttaaggtcagctccaactatccatgaaatgtgtgcaatttttgatatatctgtcgaggatcatttggtaagaattgaagacaattatatcttgacactgcatagaatcccaccgatttctaaaaacagatttaacaataaagtggtatacttacatcacggtcttttgatgtgttctgatgtttggtgttgcaatattgaaagacataaaaacttaccgtttgtattgcatgatttaggttacgacgtctggatgggaaataatagaggtaataaatattcaactgcccacttgaacaaaccaccaaaatcgaacaagttttgggatttttctatcgacgaatttgcgttctttgacattccaaactcaattgaattcatcttagatataacaaaagtggacaaggtcatctgcatcggattctctcaaggctctgctcaaatgtttgctgcattttcgttgagtgaaaaattgaatcgaaaagtctcccattttatagccatagcacccgctatgactccaaaggggttgcacaacagaattgtcgataccttggccaaatcatcgcccggctttatgtatcttttctttggtaggaaaattgtgttaccttcagctgtcatttggcaaagaactttacatccaacacttttcaatttgtgtatcgatatcgcaaacaagatactgttcaattggaagtcgtttaacattctaccgagacaaaagattgcttcttacgcaaaactttattcaacgaccagtgtaaaatccattgttcactggttccaaatattaagatctcagaaatttcaaatgtttgaagagtctgataacatgctaaattccttaactaggccttaccaaattgctaattttcctactagaacaaatatcaagattcccattcttttaatttatggtggcatagattctttagttgatattgatgtgatgaaaaaaaatctacccttcaactccgtctttgatgttaaagttgacaattatgaacacctggatttgatttggggcaaagatgccgataccttggtcattgctaaagtcttgaggtttattgaattttttaaccctggtaatgtttcagtgaagactaaccagttactaccatcagcaagtctggttgaggaattaccaagcacgacatggaagacaactcatccaacgcatggtctcagttatagaactcactcggcagaccgttctccgttgtctgtacaagctgatgaagcggatgaggtccacaatgctgacaattcaaggttcttaagacgagtgttttctactagtgccatagacgaggacaacgaaaatgagcaccaggatgatacagaagatcaaatccataaggagcagcaaagacggttaagtgtctatctggaatcatccaaagatttacgacaactagatgccaactcttcgacaactgcgctggatgctctaaataaagaatga

SEQ ID NO: 40 - protein, 548 aa, Saccharomyces cerevisiae
MYFPFLGRLSITDYIIVVLVYIESIISSVLKLIPQPMINLFEWLINFSTSSDDNTIEEKLRSAPTIHEMCAIFDISVEDHLVRIEDNYILTLHRIPPISKNRFNNKVVYLHHGLLMCSDVWCCNIERHKNLPFVLHDLGYDVWMGNNRGNKYSTAHLNKPPKSNKFWDFSIDEFAFFDIPNSIEFILDITKVDKVICIGFSQGSAQMFAAFSLSEKLNRKVSHFIAIAPAMTPKGLHNRIVDTLAKSSPGFMYLFFGRKIVLPSAVIWQRTLHPTLFNLCIDIANKILFNWKSFNILPRQKIASYAKLYSTTSVKSIVHWFQILRSQKFQMFEESDNMLNSLTRPYQIANFPTRTNIKIPILLIYGGIDSLVDIDVMKKNLPFNSVFDVKVDNYEHLDLIWGKDADTLVIAKVLRFIEFFNPGNVSVKTNQLLPSASLVEELPSTTWKTTHPTHGLSYRTHSADRSPLSVQADEADEVHNADNSRFLRRVFSTSAIDEDNENEHQDDTEDQIHKEQQRRLSVYLESSKDLRQLDANSSTTALDALNKE

SEQ ID NO: 41 - DNA, 1206 bp, Synechocystis sp. pcc6803
  CDS (1)..(1206): succinate-CoA ligase (SLL1023)
atggacttgttagagtaccaggccaaggaactatttcagcaagtgggcattcccattttgccttcccagactatccaaaacaccactgccctcaaacggttgcaaattccctaccccatggtgctcaaatcccaggtgcgggcggggggccggggtaaggccggcggggttaggtttgtggaaaataccattgatgccattgccgccgcttcggccattttccatttgcccattgcggatgagtacccagaggtgattttggctgaggcccgctacgatgcccagtcggaattatttttggcgatcgtgctggactaccaacggcagtgccctgtgttaatgggttccagcgaggggggcattgatgtggaaaccctgttggagcaaatgcagagcgtttccctgcggactaacttttctccctatctagcccgacggctagcggtgaaaatgggtttgaccgggcccctggtgactgcggtgagcggcatcatcggcaaaatgtatgaattgtttgtcacctatgacttggacgtgattgaaattaatcccctgggtattagtgctgacggggaggtgatggccctggacggcaaaattacagtcaacgatacggcgatcaaccggcatccggatttgatcaattggcgatcggagcaatggacggggcatagttggctgccggggaacttggcccagggacaaattggcctaatttgtaacagtgaagggctagccctgagtacgtgggacctgctcaattccttcggcatcaccggcgcctatttattggatgaaggtcgcagtgacataaccctgggggaacaactggaattggcctttaaccacctcagccaggcccccaatttaaaaggaatttttgttaacctggccaccagagcaacggacacctcggccttggcggaagatttacggtcttttttacccctgccccccaacctctccagtgaagaccgcagtttacgggggacgggccccagtttgccccagcgccaacgggttccccaacggcaaacctatacaggggaagtgctaccggtggttatccgatttagccagggaaatttagatcatctccagcagatgtacgacaacagccttgtccattggcacaatgatttggaaacggcgatcgccaagatgctgagtttgatgccccaagagacggcattggcccagggataa

SEQ ID NO: 42 - protein, 401 aa, Synechocystis sp. pcc6803
MDLLEYQAKELFQQVGIPILPSQTIQNTTALKRLQIPYPMVLKSQVRAGGRGKAGGVRFVENTIDAIAAASAIFHLPIADEYPEVILAEARYDAQSELFLAIVLDYQRQCPVLMGSSEGGIDVETLLEQMQSVSLRTNFSPYLARRLAVKMGLTGPLVTAVSGIIGKMYELFVTYDLDVIEINPLGISADGEVMALDGKITVNDTAINRHPDLINWRSEQWTGHSWLPGNLAQGQIGLICNSEGLALSTWDLLNSFGITGAYLLDEGRSDITLGEQLELAFNHLSQAPNLKGIFVNLATRATDTSALAEDLRSFLPLPPNLSSEDRSLRGTGPSLPQRQRVPQRQTYTGEVLPVVIRFSQGNLDHLQQMYDNSLVHWHNDLETAIAKMLSLMPQETALAQG

SEQ ID NO: 43 - DNA, 786 bp, Synechocystis sp.
  CDS (1)..(786): cobalt-precorrin-6A reductase (SLR0252)
atggtggatagcctggcaaccgtttggttaattggtggaacggtggatagtcgtgccgtagccgaagggttaattgcccagggaataaattgtctggtgacagtcaccacatcggaggccaagcatttatatccgattcatcaatgtttgacggttcacgttggagctttaaccccacaggaaataccgaaatttctaaaacgccatagcattgcagtcattgtcgatgcgtcccatccctttgccgcccaaattactaccacggtcacggcgatcgccaaagaacaacaaattccctatatccgatttgagcggccgcccctggctttgggaaaaaataccttagaagtacctgatattcagagtttaaccagagggaaatatcagccttacttgaggggaaaacgggtgctgttgaccgttggagcccgctggttaagccatttcagcctcctacaggatgaagcagttctatttgcccgtattctgccctatccccaagccctagcccaggcgatcgccgctgggtttacttccgaccgtatcattgctttgcgcccccccgtagccgaaccattggaaaaagccctgtggcaacaatggcaaattcaaggggtggtaactaaagcctccggtgcccaggggggagaattggttaagcaaaaagtggcggaagcgttgggggtaaatctgatcagaattgcccgtccccagactattccagggcaaataactgacgatttaagccagatcaaccaattttgccaaagacatttgccaagctaa

SEQ ID NO: 44 - protein, 261 aa, Synechocystis sp.
MVDSLATVWLIGGTVDSRAVAEGLIAQGINCLVTVTTSEAKHLYPIHQCLTVHVGALTPQEIPKFLKRHSIAVIVDASHPFAAQITTTVTAIAKEQQIPYIRFERPPLALGKNTLEVPDIQSLTRGKYQPYLRGKRVLLTVGARWLSHFSLLQDEAVLFARILPYPQALAQAIAAGFTSDRIIALRPPVAEPLEKALWQQWQIQGVVTKASGAQGGELVKQKVAEALGVNLIRIARPQTIPGQITDDLSQINQFCQRHLPS

SEQ ID NO: 45 - DNA, 1182 bp, Escherichia coli
  CDS (1)..(1182): uroporphyrin III C-methyltransferase (b3803)
atgacggaacaagaaaaaacctccgccgtggttgaagagaccagggaggccgtggacaccacgtcacaacctgtcgcaacagaaaaaaagagtaagaacaataccgcattgattctcagcgcggtggctatcgctattgctctggcggcgggcatcggtttgtatggctggggtaaacaacaggccgtcaatcagaccgccaccagcgatgccctggctaaccaactgacggcattgcaaaaagcccaggagagccaaaaagccgagctggaaggcattattaagcaacaagctgcacaacttaagcaggcgaatcgtcagcaagaaacgctggcaaaacagttggatgaagtccaacaaaaggtcgccaccatttccggcagcgatgctaaaacctggctgctggctcaggccgattttctggtgaaactcgccggacggaagctgtggagcgatcaggacgtcacgaccgctgcagcgttgctgaaaagtgcagacgccagcctggcggatatgaatgacccgagtctgattaccgttcgtcgggcaattaccgatgatatcgccagcctttctgcagtatcgcaggtggattatgacggcatcatccttaagcttaatcagctttcaaatcaggtagataacctgcgtctggccgataatgacagcgatggttcgccgatggattcagacggtgaagagctttccagttccatcagcgaatggcgtatcaatctgcaaaaaagctggcagaactttatggacaacttcattacgattcgccgtcgtgatgacaccgccgtaccgctgttagcgccaaatcaggatatctatctgcgcgaaaatattcgctctcgcctgctggtcgcagcacaagctgtaccgcgtcaccaggaagagacttatcgccaggcgctggagaacgtctccacctgggtacgtgcttactacgatactgatgatgccaccaccaaagcgttcctcgacgaggtggaccagttaagccagcaaaatatctcgatggatcttccggaaaccctgcaaagccaggcgatgctggaaaaactgatgcagactcgcgtgcgtaacctgctggcacaaccggcagcggggacaacggaagctaaacctgcacctgcaccgcaagctgatactccggcagccgcgccgcaaggagaataa

SEQ ID NO: 46 - protein, 393 aa, Escherichia coli
MTEQEKTSAVVEETREAVDTTSQPVATEKKSKNNTALILSAVAIAIALAAGIGLYGWGKQQAVNQTATSDALANQLTALQKAQESQKAELEGIIKQQAAQLKQANRQQETLAKQLDEVQQKVATISGSDAKTWLLAQADFLVKLAGRKLWSDQDVTTAAALLKSADASLADMNDPSLITVRRAITDDIASLSAVSQVDYDGIILKLNQLSNQVDNLRLADNDSDGSPMDSDGEELSSSISEWRINLQKSWQNFMDNFITIRRRDDTAVPLLAPNQDIYLRENIRSRLLVAAQAVPRHQEETYRQALENVSTWVRAYYDTDDATTKAFLDEVDQLSQQNISMDLPETLQSQAMLEKLMQTRVRNLLAQPAAGTTEAKPAPAPQADTPAAAPQGE

SEQ ID NO: 47 - DNA, 1107 bp, Brassica napus
  CDS (1)..(1107): uroporphyrin III C-methyltransferase (BN51286476)
atggctcttgtccagcggattcccagtttctcatccaatcttcgaaactggaagagcaccaattcgacaaaccataagcctgtttcttctctccattacaaaactcagatcactgcttcttcttcctcgcccttcacggagaagcactctgttgagagataccaaagagatcaatggctgtacaaagcatccactccatctccatctccatcgccgccgtcgaatcagcaagatgaagtctttgttagggaaaacgacatcgcatcgcagctgcctgagctgaagaagctcttggaggttctgagagagaagagagagagtggatgcagaggcggtgattgcggaccaggagatgtgtttctggtcgggacaggaccgggagatcctgagcttttgactttgaaagctgtcagagtcattcaaagcgctgatcttctgctttacgacaggcttgtctctaatgatgttctggagttggttgctcctgatgctaggcttctctatgtcggcaaaactgctggctatcatagcagaactcaggaggagatacatgaactactccttagttttgctgaagctggtgctactgttgtgaggcttaaaggtggagatcctttggtttttggacgaggtggggaagagatggactttctgcaacaacaagggattcgagttaaagttatcccagggattacagcggcgtctgggatagcagcagagcttgggattcccttaacgcatcgaggtgttgcaactagtgtgaggttcctcactggtcattccaggaaaggagggaccgatcctctcttcgtcgcagagaatgcagctgatcctgacacgacacttgttgtttatatgggtttgggaactttaccttctcttgcgcagaaactgatggaccacggtctaccttgtgacacaccagctgttgcggttgaacgtggaaccactcctcttcagcgtaatgtttttgctgagcttaaagactttgcgactgagattcaggcagctggattggtgtcaccaacgctcatcatcatagggaaagtcgtcgagctatctcctttatggcctcattgcacgaaggaatactttttgcttgtttaa

SEQ ID NO: 48 - protein, 368 aa, Brassica napus
MALVQRIPSFSSNLRNWKSTNSTNHKPVSSLHYKTQITASSSSPFTEKHSVERYQRDQWLYKASTPSPSPSPPSNQQDEVFVRENDIASQLPELKKLLEVLREKRESGCRGGDCGPGDVFLVGTGPGDPELLTLKAVRVIQSADLLLYDRLVSNDVLELVAPDARLLYVGKTAGYHSRTQEEIHELLLSFAEAGATVVRLKGGDPLVFGRGGEEMDFLQQQGIRVKVIPGITAASGIAAELGIPLTHRGVATSVRFLTGHSRKGGTDPLFVAENAADPDTTLVVYMGLGTLPSLAQKLMDHGLPCDTPAVAVERGTTPLQRNVFAELKDFATEIQAAGLVSPTLIIIGKVVELSPLWPHCTKEYFLLV

SEQ ID NO: 49 - DNA, 1092 bp, Glycine max
  CDS (1)..(1092): uroporphyrin III C-methyltransferase (GM59791864)
atggctctcctccacaagctcgcacctttcacctctctctacaccaataccccccattccccaccaccccgccgccacgtcatcatctccttctccgtctcacccttcaccgagaagcactccacagagcgctaccagagggaccaatgggtctaccagagcaccactcaagagggtcaacctcaaacgacgccgtttcctcctctcccgtgcgattcagcgtccttaagagacgacgacatagcgttgcagctgccagagctgaagaagctgcttcaggttctgagggagaagagggagtgtatcaatggagagggttgtgagccggggaatgtgttcttggtgggaacgggacctggggaccctgagcttctcactctcaaggctgtgagagtcatcaagagtgctgatcttttgctctatgacaggttggtgtccaatgatgtgctggatttggttggccccggtgctaggcttctctatgtgggcaagactgctgggtatcatagcagaacacaggaggagatacatgagcttcttttgagttttgcagaagctggggcaactgttgtgagacttaaagggggtgatccattggtgtttgggaggggtggggaagaaatggattttttgcaacagcaaggtatccaagtgaaagttattccaggtataactgctgcatctggaatagcagcagagcttggaattccattaactcaccgtggcattgcaaatagtgtgagattcctcacagggcattcaaggaaaggaggatccgatcctctctttgtatcggaaaatgctgctgatccggattctaccttggtggtttacatgggcttggcgaccttcccttcacttgcacagaagctaatgcatcatggtctgtcccctcagaccccagctgcagccattgagcgaggaaccacacttcatcagcgcacggtgtttgctgaactaaaggaccttcacgagaaaattacatctgctcagctggtgtcaccaacgctgattattataggaaaagttgttgaactatcaccattttggccaattcctacaaaagaagaatcatgtttaatgcaggcatga

SEQ ID NO: 50 - protein, 363 aa, Glycine max
MALLHKLAPFTSLYTNTPHSPPPRRHVIISFSVSPFTEKHSTERYQRDQWVYQSTTQEGQPQTTPFPPLPCDSASLRDDDIALQLPELKKLLQVLREKRECINGEGCEPGNVFLVGTGPGDPELLTLKAVRVIKSADLLLYDRLVSNDVLDLVGPGARLLYVGKTAGYHSRTQEEIHELLLSFAEAGATVVRLKGGDPLVFGRGGEEMDFLQQQGIQVKVIPGITAASGIAAELGIPLTHRGIANSVRFLTGHSRKGGSDPLFVSENAADPDSTLVVYMGLATFPSLAQKLMHHGLSPQTPAAAIERGTTLHQRTVFAELKDLHEKITSAQLVSPTLIIIGKVVELSPFWPIPTKEESCLMQA

SEQ ID NO: 51 - DNA, 1140 bp, Zea mays
  CDS (1)..(1140): uroporphyrin III C-methyltransferase (ZMBFb0243J04)
atggctctcgccgtccgcacgcctcggttccagcctcgaccggcctccatttctgcgcccacaacacccgcatcgacctccctcgccgccgggaacgcgaggccccgcccctgcgccggcgccgccgttcgcgcctcgcccttcaccgaggccacctcctcctcgcgctaccgccgcgacgcctggtcatacgccgccgatggctcctccaactccgcatccccgtcctccgctgccgctgctgccgcgggccgtcgggatgacgagatcgcgctgcagctgccggaactgcggaggctcctggacgtgctgagggcgtccagggggaggggcgccgaggagggcggcggacccgggagggtggcactggtgggcacagggcccggggaccccgagctgctcacgctcaaggcggtccgggccatcgaggcggcggacctggtgctctacgacaggctggtgtctaacgacgtgttggacttggtcggggagggcgccaggctgctctatgtcggcaagaccgcgggataccacagccgcacccaggaggagattcacgagctgctgctgagctttgcggaggccggtgccaatgttgtgcgcttgaaaggtggcgatcctctggtttttggaaggggtggagaagagatggatttcttacagcaacagggaattaaagttgaagttatcccaggaattacttctgcttcgggtattgcagcagagctaggcattccgttaacccatagaggtgttgctaccagtgtcagatttttaactggccactccagaaatggtgggacagatccactatatgtagctggaaatgcggctgatccagacactactttggttgtgtatatgggtttgtcaacacttccatcacttgctccaaagttaatgaagcacggcctgccgccagatacccctgccgttgcagtagagcgcgggactacacctcaacaacgaacggtgttcgcctcgttgaaggatcttgtggatgaagtcaagtcagccgatctagtttctccaactctaataattattgggaaagtggtatctttgtcacccttctgggttgagtcatctgaacatgatgcactgaaagttcagagctcgtatgcaaatgaggccagatga

SEQ ID NO: 52 - protein, 379 aa, Zea mays
MALAVRTPRFQPRPASISAPTTPASTSLAAGNARPRPCAGAAVRASPFTEATSSSRYRRDAWSYAADGSSNSASPSSAAAAAAGRRDDEIALQLPELRRLLDVLRASRGRGAEEGGGPGRVALVGTGPGDPELLTLKAVRAIEAADLVLYDRLVSNDVLDLVGEGARLLYVGKTAGYHSRTQEEIHELLLSFAEAGANVVRLKGGDPLVFGRGGEEMDFLQQQGIKVEVIPGITSASGIAAELGIPLTHRGVATSVRFLTGHSRNGGTDPLYVAGNAADPDTTLVVYMGLSTLPSLAPKLMKHGLPPDTPAVAVERGTTPQQRTVFASLKDLVDEVKSADLVSPTLIIIGKVVSLSPFWVESSEHDALKVQSSYANEAR

SEQ ID NO: 53 - DNA, 663 bp, Escherichia coli
  CDS (1)..(663): isoprenoid biosynthesis protein (b3209)
atgatcacaatgaagaaaattggcgtaattctgagcggatgcggcgtctatgacggttctgaaattcatgaagcggtgttgacgttgttagctatttcacgcagcggtgcgcaggcggtctgctttgcaccggataagcagcaggttgatgttatcaaccatttaactggcgaagcgatgacggaaacgcgcaatgtgctgattgaagcggcacgaataacgcgcggtgaaatccgtcctctggcccaggccgatgccgctgaactggatgcgttgattgtgccgggggggtttggcgcggcgaagaatttaagcaattttgccagtcttggtagcgaatgcaccgttgaccgtgaattaaaggcgctggcacaagcgatgcatcaggccggaaaaccgcttggttttatgtgtattgccccggcgatgctgccgaaaattttcgatttcccgctgcgtttgaccatcggtactgatatcgataccgcagaagtgctggaagagatgggcgcggagcatgtgccgtgtcctgtcgatgatatcgtggttgatgaagacaataagattgtcaccaccccagcatatatgctggcgcagaacattgcagaagcggcgagcggcattgataagctggtttcccgcgtgctggttcttgctgaatga

SEQ ID NO: 54 - protein, 220 aa, Escherichia coli
MITMKKIGVILSGCGVYDGSEIHEAVLTLLAISRSGAQAVCFAPDKQQVDVINHLTGEAMTETRNVLIEAARITRGEIRPLAQADAAELDALIVPGGFGAAKNLSNFASLGSECTVDRELKALAQAMHQAGKPLGFMCIAPAMLPKIFDFPLRLTIGTDIDTAEVLEEMGAEHVPCPVDDIVVDEDNKIVTTPAYMLAQNIAEAASGIDKLVSRVLVLAE

SEQ ID NO: 55 - DNA, 693 bp, Glycine max
  CDS (1)..(693): isoprenoid biosynthesis protein (GMss34d01)
atggctccgaagaaggttcttctcctttgcggggacttcatggaagactatgaagccatggttccatttcaggcattgcaagccttcggtctcgccgtcgacgccgtctgtcccggaaagaaatccggcgatgtctgccgcaccgctgtccatgtacttgccggcgcccagacttatagtgagacggttggtcacaatttttcactcaatgcaacgtttgatgaagttgatgctgcaagctatgatgggttgtgggtgccaggtgggagggctccagagtatcttgctcatgttcctggtgttgtggaactggtgaccaagtttgtcagtttgggaaaacaaattgctagcatttgtcatggacagttgattctggctgctgctggagtggttaaaggccgcacgtgcacggcttttcctcctgttaaacccgtgctagttgctgctggtgctcattgggttgaacctgacactgaggcagcaacagtggtagatggtgatctcattactgcagctacttatgaggggcacccggagctcattcgccatttcgtgaaggcattaggaggcaaaataagtggttttgataagaaaatcctctttatttgtggggattacatggaagattatgaggtcaaggttccttttcagtcccttcaggctttaggatgccatgttgatgctgtttgcccc

SEQ ID NO: 56 - protein, 231 aa, Glycine max
MAPKKVLLLCGDFMEDYEAMVPFQALQAFGLAVDAVCPGKKSGDVCRTAVHVLAGAQTYSETVGHNFSLNATFDEVDAASYDGLWVPGGRAPEYLAHVPGVVELVTKFVSLGKQIASICHGQLILAAAGVVKGRTCTAFPPVKPVLVAAGAHWVEPDTEAATVVDGDLITAATYEGHPELIRHFVKALGGKISGFDKKILFICGDYMEDYEVKVPFQSLQALGCHVDAVCP

SEQ ID NO: 57 - DNA, 486 bp, Helianthus annuus
  CDS (1)..(486): isoprenoid biosynthesis protein (HA03MC1392)
atgatatgtggcgattacatggaagactctgaggcgatggttccgtttcaggcattgcaagcttacggattatccgtcgatgctgtttgccctggtaaaaagtccggcgatatttgtcgcactgctattcatcagggttctcctcaccagacatattccgagtcgcgtggtcacaacttcactctcaatgccacgtttgatgaaattgacgcaaacacatatgacggattggttctaccaggaggacgtgctccagaatatctcgctatgaatgaatcagttcttaacctggtcacacattttgcaaaatctggaaaacccattgcctccatttgtcatgggcaacttatattggcggctgctgctgctaaagctgatgtaataaaaggtcgaaaagttacagcatatgcgactgtgggacccgtagcttgctgctgctggagctcattgggtacaccccgagacattggcatcatgtaccggtga

SEQ ID NO: 58 - protein, 161 aa, Helianthus annuus
MICGDYMEDSEAMVPFQALQAYGLSVDAVCPGKKSGDICRTAIHQGSPHQTYSESRGHNFTLNATFDEIDANTYDGLVLPGGRAPEYLAMNESVLNLVTHFAKSGKPIASICHGQLILAAAAAKADVIKGRKVTAYATVGPVACCCWSSLGTPRDIGIMYR

SEQ ID NO: 59 - DNA, 588 bp, Escherichia coli
  CDS (1)..(588): LysE-type translocator (b2578)
atgacaccgacccttttaagtgctttttggacttacaccctgattaccgctatgacgccaggaccgaacaatattctcgcccttagctctgctacgtcgcatggatttcgtcaaagtacccgcgtgctggcagggatgagtctgggatttttgattgtgatgttactgtgtgcgggcatttcattttcactggcagtgattgacccggcagcggtacaccttttgagttgggcgggggcggcatatattgtctggctggcgtggaaaatcgccaccagcccaacaaaggaagacggacttcaggcaaaaccaatcagcttttgggccagctttgctttgcagtttgtgaacgtcaaaatcattttgtacggtgttacggcactgtcgacgtttgttctgccgcaaacacaggcgttaagctgggtagttggcgtcagcgttttgctggcgatgattgggacgtttggcaatgtgtgctgggcgctggcggggcatctgtttcagcgattgtttcgccagtatggtcgccagttaaatatcgtgcttgccctgttgctggtctattgcgcggtacgcattttctattaa

SEQ ID NO: 60 - protein, 195 aa, Escherichia coli
MTPTLLSAFWTYTLITAMTPGPNNILALSSATSHGFRQSTRVLAGMSLGFLIVMLLCAGISFSLAVIDPAAVHLLSWAGAAYIVWLAWKIATSPTKEDGLQAKPISFWASFALQFVNVKIILYGVTALSTFVLPQTQALSWVVGVSVLLAMIGTFGNVCWALAGHLFQRLFRQYGRQLNIVLALLLVYCAVRIFY

SEQ ID NO: 61 - DNA, 738 bp, Escherichia coli
  CDS (1)..(738): LIV-E family branched-chain amino acid transporter (b2382)
atggaaagccctactccacagcctgctcctggttcggcgaccttcatggaaggatgcaaagacagtttaccgattgttattagttatattccggtggcctttgcgttcggtctgaatgcgacccgtctgggattctctcctctcgaaagcgtttttttctcctgcatcatttatgcaggcgcgagccagttcgtcattaccgcgatgctggcagccgggagtagtttgtggattgctgcactgaccgtcatggcaatggatgttcgccatgtgttgtatggcccgtcactgcgtagccgtattattcagcgtctgcaaaaatcgaaaaccgccctgtgggcgtttggcctgacggatgaggtttttgccgccgcaaccgcaaaactggtacgcaataatcgccgctggagcgagaactggatgatcggcattgccttcagttcatggtcatcgtgggtatttggtacggtaataggggcattctccggcagcggcttgctgcaaggttatcccgccgttgaagctgcattaggttttatgcttccggcactctttatgagtttcctgctcgcctctttccagcgcaaacaatctctttgcgttaccgcagcgttagttggtgcccttgcaggcgtaacgctattttctattcccgtcgccattctggcaggcattgtctgtggctgcctcactgcgttaatccaggcattctggcaaggagcgcccgatgagctataa

SEQ ID NO: 62 - protein, 245 aa, Escherichia coli
MESPTPQPAPGSATFMEGCKDSLPIVISYIPVAFAFGLNATRLGFSPLESVFFSCIIYAGASQFVITAMLAAGSSLWIAALTVMAMDVRHVLYGPSLRSRIIQRLQKSKTALWAFGLTDEVFAAATAKLVRNNRRWSENWMIGIAFSSWSSWVFGTVIGAFSGSGLLQGYPAVEAALGFMLPALFMSFLLASFQRKQSLCVTAALVGALAGVTLFSIPVAILAGIVCGCLTALIQAFWQGAPDEL

SEQ ID NO: 63 - DNA, 309 bp, Escherichia coli
  CDS (1)..(309): DNA-binding protein (b3285)
atgacggaaccggaagaaattctggaaaacttgcaatttggattgcactggttgccagacgcccctgaaaattcattttattcaccagatcagcaagacgtggcattgccatttcctgagctcctggctaacgtaggagatgaggtaacacctgttgacgtcgtcgctgaacgtgccggccaacctgtgccagaggtagttactcaactactcgaactggagttagcaggatggatcgcagctgtacccggcggctatgtccgattgaggagggcatgccatgttcgacgtactaatgtatttgtttaa

SEQ ID NO: 64 - protein, 102 aa, Escherichia coli
MTEPEEILENLQFGLHWLPDAPENSFYSPDQQDVALPFPELLANVGDEVTPVDVVAERAGQPVPEVVTQLLELELAGWIAAVPGGYVRLRRACHVRRTNVFV

SEQ ID NO: 65 - DNA, 1659 bp, Escherichia coli
  CDS (1)..(1659): FLiF protein (b1938)
atgaatgcgactgcagcccagacaaaatctcttgagtggcttaatcgcctgcgtgcgaatccgaaaattccattgattgttgccggttccgcggcagtggcggtcatggtcgcactgatcctgtgggcgaaagcccccgactaccgcacattattcagcaatctttccgatcaggatggtggcgcaattgtcagccaactgacgcaaatgaatattccttaccgcttcagcgaagccagcggcgctattgaagttccggcagataaagttcacgaactgcgtctgcgcctggcacaacaaggtttgccaaaaggcggcgcggtcggtttcgaactgcttgatcaggaaaagtttggtatcagccagttcagcgaacaggtgaattatcagcgggcgctggaaggcgagctttctcgtaccatcgaaactatcggcccggtaaaaggggcgcgcgtacatctggcaatgccgaaaccgtctttattcgtccgtgaacaaaaatccccttctgcatcggtgacggtaaatctgttacccggccgcgcactcgatgaagggcaaattagcgccattgtgcatctggtttccagcgccgttgctggtctgccgccgggaaacgtcacgctggtggatcagggcggacatctgttaacccagtccaataccagcgggcgcgatcttaatgacgctcagttgaaatatgccagcgatgtcgaaggccgtattcagcggcgtattgaagcgatcctgtcgcctattgttggtaacggtaatattcacgcccaggttacggcgcagctggacttcgccagtaaagaacaaacggaagaacagtatcgccctaacggtgatgaatctcatgcggcgcttcgttcacgccagcttaatgagagcgagcaaagcggttccggttatccgggcggcgtaccgggggcgttgtcgaatcaaccggcacctgcgaataacgcgccaatcagcacgcctccggcaaatcaaaataaccgccagcagcaggcgagcaccaccagcaatagtgggccgcgtagcacacagcggaatgaaaccagtaactacgaagtcgatcgcaccattcgtcataccaaaatgaacgtgggcgatgtgcaacgtctgtcagtcgcggtcgtggtgaattacaaaaccttgccagatggcaaaccgttgcctctcagcaacgaacagatgaagcaaattgaagatctgacccgcgaggcgatgggcttttctgaaaaacgcggtgactcgctcaatgtcgttaactcgccgttcaatagcagtgacgaaagcggcggagaactgccattctggcaacagcaagcgtttatcgatcagttacttgctgccggtcgctggttgctggtactgctggtggcgtggctgctgtggcggaaagcggtacgtccgcagctaacacgtcgcgctgaggcgatgaaagctgtacagcaacaggcgcaggcccgcgaggaagtggaagatgcggtggaagtccgcctgagcaaagacgaacaactacaacaacggcgcgctaaccaacgtctgggggcagaagtcatgagccagcgtatccgtgaaatgtctgataacgatccgcgcgtggtggcgctggtcattcgccagtggataaataacgatcatgagtaa

SEQ ID NO: 66 - protein, 552 aa, Escherichia coli
MNATAAQTKSLEWLNRLRANPKIPLIVAGSAAVAVMVALILWAKAPDYRTLFSNLSDQDGGAIVSQLTQMNIPYRFSEASGAIEVPADKVHELRLRLAQQGLPKGGAVGFELLDQEKFGISQFSEQVNYQRALEGELSRTIETIGPVKGARVHLAMPKPSLFVREQKSPSASVTVNLLPGRALDEGQISAIVHLVSSAVAGLPPGNVTLVDQGGHLLTQSNTSGRDLNDAQLKYASDVEGRIQRRIEAILSPIVGNGNIHAQVTAQLDFASKEQTEEQYRPNGDESHAALRSRQLNESEQSGSGYPGGVPGALSNQPAPANNAPISTPPANQNNRQQQASTTSNSGPRSTQRNETSNYEVDRTIRHTKMNVGDVQRLSVAVVVNYKTLPDGKPLPLSNEQMKQIEDLTREAMGFSEKRGDSLNVVNSPFNSSDESGGELPFWQQQAFIDQLLAAGRWLLVLLVAWLLWRKAVRPQLTRRAEAMKAVQQQAQAREEVEDAVEVRLSKDEQLQQRRANQRLGAEVMSQRIREMSDNDPRVVALVIRQWINNDHE

SEQ ID NO: 67 - DNA, 1671 bp, Synechocystis sp. PCC 6803
  CDS (1)..(1671): GTP cyclohydrolase II (SLL1894)
atgtttgatgccattgatgcagccctagctgacattaaagcgggcaaggccgtggtggtggtggacgacgaaaatcgagaaaatgaaggggatttaatctgtgcggcccaatttgccacccctgctctagttaactttatggcagtggaagcccggggcttgatttgcctagccatgactggtgatcgcctggacgaattggatctgcccttgatggtcagtaaaaacaccgacagcaaccaaaccgctttcaccgtcagcattgatgcagccccccatttgggcgtaaccacagggatttccgccgaagaccgggcccggacgatccagattgccattaaccccgtcacccgcccggaggacctttcccgacctggccatattttccctctgcgggccaaaactgggggagtactgaaacgagccggccacaccgaggcggcagtcgatctttctcgcctagcaggactctatccagcgggggtaatttgcgaaattcaaaatgccgatggctccatggcccgcctaccggaattggtggagtatgcccgcaaatatgacctcaaactgatcagcattgccgatttaatcagttaccgactccaacatgaccgctttgtgcaacgggaaaccatctgtgaatttcccagccagtttggggagtttaaactctacgcctaccgcaatttattggatcaaacagagcacattgccattgtgaagggtgatcctagtgaatttggtcaacagccggtgatggtgcggatgcattcagaatgtttaacgggggatgccctgggttctctccgttgtgattgccgcatgcaactccaggcggccctcaaaatgctggaaaaccatggtttgggggtggtggtttatctccgccaggaaggccggggtattggtttggtcaataagctcaaggcctattccctacaggatttgggctatgacaccgtggaagcgaatgaaagattaggttttccggcggatctgcgggactacggcatgggggcgcaaatgctcaacgatttaggggtgaaacaaatccgtttgatcaccaacaatccccgtaaaattgccggactgaaaggctacggtctagaaattgtcgaacgggtgcccctgttaattgaggccaacgactataactcccattacctgaccaccaaggcagaaaaactagggcactggttagtcaaaaactatctgctggcgatcgccattaaatttactcccaacgttgcttcggcccaacaacggtacgaaaaattggaaaaaatccgggccctgttacagggaacccccctcatggtgcatgaagacaaccgccccgtggcgatcgccctatttggcaaaaatagcctaattttccacgtcggcctagaccaaaacttaccatcgggccagccttggcaaaagcacgaccattctccctaccccgctttggtaaaggattttctcgtacagctcaaacaatggccagacctacaggctctagcattcctcatcgcccaagggaaagaccccatggaaactctccaagtcagtctggaccgggaaaatgtatcctttgccgacctcaccgccgacgccctaaaccaatggcagccccaaacggtgtacacctacagcaagcaaggcagcggagaaatgactaacagataa

SEQ ID NO: 68 - protein, 556 aa, Synechocystis sp. PCC 6803
MFDAIDAALADIKAGKAVVVVDDENRENEGDLICAAQFATPALVNFMAVEARGLICLAMTGDRLDELDLPLMVSKNTDSNQTAFTVSIDAAPHLGVTTGISAEDRARTIQIAINPVTRPEDLSRPGHIFPLRAKTGGVLKRAGHTEAAVDLSRLAGLYPAGVICEIQNADGSMARLPELVEYARKYDLKLISIADLISYRLQHDRFVQRETICEFPSQFGEFKLYAYRNLLDQTEHIAIVKGDPSEFGQQPVMVRMHSECLTGDALGSLRCDCRMQLQAALKMLENHGLGVVVYLRQEGRGIGLVNKLKAYSLQDLGYDTVEANERLGFPADLRDYGMGAQMLNDLGVKQIRLITNNPRKIAGLKGYGLEIVERVPLLIEANDYNSHYLTTKAEKLGHWLVKNYLLAIAIKFTPNVASAQQRYEKLEKIRALLQGTPLMVHEDNRPVAIALFGKNSLIFHVGLDQNLPSGQPWQKHDHSPYPALVKDFLVQLKQWPDLQALAFLIAQGKDPMETLQVSLDRENVSFADLTADALNQWQPQTVYTYSKQGSGEMTNR

SEQ ID NO: 69 - DNA, 1635 bp, Glycine max
  CDS (1)..(1635): GTP cyclohydrolase II (GM08000037)
atggcttctttcaatctctcttattcttcctcaccagccctctcacgtccccgggcaagcaaacacttcaaattcttcaatgatgtgccttttgtccgaatgaattctaaatttccctcaagtgataatgcttttggtagaatcaaagctatgttgatctctggagggggtgaactactttcccatccaaatggcaatgacattgtgataggtacaaatctggttggagatgaatctgttcaaattgaggtacagcctaatggaacactggcagatgacacagggctaacaagcaatgttttctctatggacaacaatgaatttgattcggacagcccctcagcaggttttgcttccattcctgaggcaattgaagacattcgccagggaaagatggtagtggttgtggacgatgaagatcgagaaaatgaaggagacttgataatggcagcacagttggcaacacccgacgcaatggcttttattgtaaagcatggaactggtatagtttgtgtaagcatgaaagaggaagatctggatagattggaacttcctttgatggtgaacagtaaggacaatgatgagaaactctgtacggcattcactgtgacagtggatgctaaacatggtacaaccactggggtgtcagctcacgatagggcaacaacagtcttggcccttgcgtctagagattctactccaggcgatttcaatcgcccgggccatattttcccactaaaatacagggaaggtggtgtcttgaagagagccggacatacagaagcttctgttgatcttgctgtacttgctggtttggatccagtggcagttctgtgtgagattgtagatgatgatggttccatggctagactgccaaagcttcgccagtttgcagagcatgaaaatttgaaaattgtatctattgctgacttaataagatatagaagaaaaagggataagttagtggatcgctcttctgctgcacagatacctacaatgtggggaccattcacagcttactgttacagatcacttttagacgggattgagcatattgcaatggttaagggtgaaattggagatggacaagatgttcttgtgagagtacactcagagtgtctgacaggagacatatttggatctgccagatgtgactgcggaaatcagcttgcacttgcaatgcaacagattgaggctgctggtaggggtgtgctggtatatctccgtggacacgaaggaaggggtattggattgggccacaagcttcgtgcttataacctacaggatgatggacgagatactgttgaagccaatgaggaattgggattgccagttgactccagggagtatggcattggtgcacagatgttgagggacttgggtgtacgatctatgaagctgatgacaaacaatccggcaaaatatattgggctcaaaggttatggtttgacagtttcgggtaggatcccattgttaactcttatcacttcggagaacaagagatatttagagaccaaacgtgtgaaaatgggtcacatatatggcacggaatttaacggccgattgagcactcatgacagtggtaatggtaatgccaccaggattgaggactctaatgctgttactggcttataa

SEQ ID NO: 70 - protein, 544 aa, Glycine max
MASFNLSYSSSPALSRPRASKHFKFFNDVPFVRMNSKFPSSDNAFGRIKAMLISGGGELLSHPNGNDIVIGTNLVGDESVQIEVQPNGTLADDTGLTSNVFSMDNNEFDSDSPSAGFASIPEAIEDIRQGKMVVVVDDEDRENEGDLIMAAQLATPDAMAFIVKHGTGIVCVSMKEEDLDRLELPLMVNSKDNDEKLCTAFTVTVDAKHGTTTGVSAHDRATTVLALASRDSTPGDFNRPGHIFPLKYREGGVLKRAGHTEASVDLAVLAGLDPVAVLCEIVDDDGSMARLPKLRQFAEHENLKIVSIADLIRYRRKRDKLVDRSSAAQIPTMWGPFTAYCYRSLLDGIEHIAMVKGEIGDGQDVLVRVHSECLTGDIFGSARCDCGNQLALAMQQIEAAGRGVLVYLRGHEGRGIGLGHKLRAYNLQDDGRDTVEANEELGLPVDSREYGIGAQMLRDLGVRSMKLMTNNPAKYIGLKGYGLTVSGRIPLLTLITSENKRYLETKRVKMGHIYGTEFNGRLSTHDSGNGNATRIEDSNAVTGL

SEQ ID NO: 71 - DNA, 495 bp, Synechocystis sp. PCC 6803
  CDS (1)..(495): lumazine synthase (SLL1282)
atgacagtttatgaagggtcgtttactcctccggcccggccatttcgttttgcccttgtaattgcccgtttcaatgatttagtaacggaaaagctactttctggttgccaagattgcctcaaacgtcatggcattgatgtggaccccgccggtacccaggtggactatatttgggttcccggtagctttgaggtgccattggtaacccgtaagttggcagtgtcggggcagtatgatgccattatctgcctaggggcggtgattcggggacaaaccccccattttgattttgtggccggagaagcggccaagggcattgcggcgatcgccagccagactggggtaccggtaatttttggcattttaaccacggataccatgcaacaggccctagaaagggcggggattaagagtaaccacggctggggctacgccatgaatgcattggaaatggctagtttgatgcgggccatggctcctttgacggaaggttaa

SEQ ID NO: 72 - protein, 164 aa, Synechocystis sp. PCC 6803
MTVYEGSFTPPARPFRFALVIARFNDLVTEKLLSGCQDCLKRHGIDVDPAGTQVDYIWVPGSFEVPLVTRKLAVSGQYDAIICLGAVIRGQTPHFDFVAGEAAKGIAAIASQTGVPVIFGILTTDTMQQALERAGIKSNHGWGYAMNALEMASLMRAMAPLTEG

SEQ ID NO: 73 - DNA, 687 bp, Glycine max
  CDS (1)..(687): lumazine synthase (GM06MC29296)
atggcttcatttgtttctacccactgtatccttcctagcggggttggatttgtgcatgaccaagatcgttcttgtggtagaactaacactttgcatcttcaaaatgctcctaaaagccctagctctctctcctttttgctctccgtgcgagaaagtagatcagatgccgttgttgttcagagcaagggtcgatcgtctttttctcaaacagctgctgtgaggcatcttacaggttctgttaccagagcccatggacttcgttttgctgtggttgttgctagatttaatgaaattgttaccaaaccgctcttggaaggtgctttgggtactttcaataattattcagttcaagatgaggatatcgatgttgtgtgggtacctggtagttttgaaattggtgttgttgcagaaaggcttggaaaatcaggcaaatatgatgcaatcgtatgcataggagctgtggtacgaggagatacaacccactatgatgcagttgctaattcagcagcatcaggagttctttcagccagtctaaactcaggtgttccatgcatatttggtgtcctaacatgtgatgatatggatcaggctctaaatagagctggtggaaagtctgggaataagggtgctgaggctgcattgactgctattgagatggcatctttgttcgagcaccatctgaagtag

SEQ ID NO: 74 - protein, 228 aa, Glycine max
MASFVSTHCILPSGVGFVHDQDRSCGRTNTLHLQNAPKSPSSLSFLLSVRESRSDAVVVQSKGRSSFSQTAAVRHLTGSVTRAHGLRFAVVVARFNEIVTKPLLEGALGTFNNYSVQDEDIDVVWVPGSFEIGVVAERLGKSGKYDAIVCIGAVVRGDTTHYDAVANSAASGVLSASLNSGVPCIFGVLTCDDMDQALNRAGGKSGNKGAEAALTAIEMASLFEHHLK

SEQ ID NO: 75 - DNA, 648 bp, Zea mays
  CDS (1)..(648): lumazine synthase (ZM07MC00430)
atggcaactgctcctgtgacatcctccatggctacgaactccacctgtgtgcggctgcctagcatgcccctgaggcgtgcacccgccatcgtctcctttgcctctcgtccatggtcaaccgcaatgacctcatgtactggattgtcgcggcactctgatgtggttgccgccgcggggcaccagaagttgatggggtctttgaacaacaacgagggactcatgtttggggtggttgtggcacggttcaacgaggtcgtgacaaatttgttactacagggtgccctggaaactttcgagcgatactctgttaaagcagaaaatataacagttgttagtgttcctggaagctttgaagttcctataacggcacaaaagcttgggaaatctggaaaatttgatgcaattctgtgcattggagctgtgattagaggtgacacaacccactatgatgccgttgcaaactcagctgcatcaggtgtactcaatgctggattatctgctggtgttccttgtgtatttggtgttctaacctgtgaagacatggatcaggcactgaaccgtgctggtggcaaggctggaaacaaaggagctgaagctgctcttactgccattgagatggcctcgctgttccggcatcacttaggctga

SEQ ID NO: 76 - protein, 215 aa, Zea mays
MATAPVTSSMATNSTCVRLPSMPLRRAPAIVSFASRPWSTAMTSCTGLSRHSDVVAAAGHQKLMGSLNNNEGLMFGVVVARFNEVVTNLLLQGALETFERYSVKAENITVVSVPGSFEVPITAQKLGKSGKFDAILCIGAVIRGDTTHYDAVANSAASGVLNAGLSAGVPCVFGVLTCEDMDQALNRAGGKAGNKGAEAALTAIEMASLFRHHLG

SEQ ID NO: 77 - DNA, 654 bp, Zea mays
  CDS (1)..(654): lumazine synthase (ZM07MC23187)
atgccggcgatggcaactgctcctgtgaaattctctgtggctacgaactccccatgtgtccagcctagcttgcccctgaggcgtacacctgccgccatctccttcccttgtcgtccatggtcagccgcaatgaactcatgtactggattgttgaggcgctctgacgtggttgcagccgcggggcaccagaagttgatgggatctttgaccactaacgagggactcaggtttggggtggttgtggcacggttcaacgaggtcgtgacaaatttgttactacagggtgccctggaaactttcgagcgatactctgttaaagaagaaaatataacagttgttagtgttcccggaagctttgaggttcctataacagcacaaaagcttgggaaatctgggaaatttgatgcaattctgtgcattggagctgtgattagaggtgacacaacccactatgatgctgttgcaaactcagctgcatcaggtgtactcaatgctggattatctgctggtgttccttgtgtatttggtgttctaacctgtgaagacatggatcaggcgctaaaccgcgctggtggcaaggctggaaacaagggtgctgaagctgctcttactgccattgagatggcctctctgttccggcatcacttaggctga

SEQ ID NO: 78 - protein, 217 aa, Zea mays
MPAMATAPVKFSVATNSPCVQPSLPLRRTPAAISFPCRPWSAAMNSCTGLLRRSDVVAAAGHQKLMGSLTTNEGLRFGVVVARFNEVVTNLLLQGALETFERYSVKEENITVVSVPGSFEVPITAQKLGKSGKFDAILCIGAVIRGDTTHYDAVANSAASGVLNAGLSAGVPCVFGVLTCEDMDQALNRAGGKAGNKGAEAALTAIEMASLFRHHLG

SEQ ID NO: 79 - DNA, 864 bp, Escherichia coli
  CDS (1)..(864): pdxY (b1636)
atgatgaaaaatattctcgctatccagtctcacgttgtttatggtcatgcgggtaacagtgcggcagagtttccgatgcgccgcctgggcgcgaacgtctggccgctgaacaccgttcaattttctaatcacacccaatacggcaaatggactggctgcgtgatgccgcccagccatttaaccgaaattgtgcaaggcattgccgccattgataaattacacacctgtgatgccgtattaagtggctatctgggatcggcggagcagggtgaacatatcctcggtatcgtccgtcaggtgaaagccgcgaatccgcaggcgaaatatttttgcgatccggtaatgggtcatccggaaaaaggctgtatcgttgcaccgggtgtcgcagagtttcatgtgcggcacggtttgcctgccagcgatatcattgcgccaaatctggttgagctggaaatactctgtgagcatgcggtaaataacgtcgaagaagcggttctggcagcgcgcgaactcattgcgcaagggccacaaattgtgttggttaaacacctggcgcgagctggctacagccgtgaccgttttgaaatgctgctggtcaccgccgatgaagcctggcatatcagccgtccgctggtggattttggtatgcgccagccggtaggtgttggtgatgtgacgagcggtttactgctggtgaaactgcttcagggggcaacgctgcaggaggcgctggaacatgtgaccgctgcagtctacgaaatcatggtgaccaccaaagcaatgcaggaatatgagctgcaagtggtggctgctcaggatcgtattgccaaaccagaacattacttcagcgcaacaaagctctga

SEQ ID NO: 80 - protein, 287 aa, Escherichia coli
MMKNILAIQSHVVYGHAGNSAAEFPMRRLGANVWPLNTVQFSNHTQYGKWTGCVMPPSHLTEIVQGIAAIDKLHTCDAVLSGYLGSAEQGEHILGIVRQVKAANPQAKYFCDPVMGHPEKGCIVAPGVAEFHVRHGLPASDIIAPNLVELEILCEHAVNNVEEAVLAARELIAQGPQIVLVKHLARAGYSRDRFEMLLVTADEAWHISRPLVDFGMRQPVGVGDVTSGLLLVKLLQGATLQEALEHVTAAVYEIMVTTKAMQEYELQVVAAQDRIAKPEHYFSATKL

SEQ ID NO: 81 - DNA, 1143 bp, Escherichia coli
  CDS (308)..(964): Pyridoxal 5′-phosphate synthase (pdxH)
atgcaaaccgatacgctggaataccagtgtgatgaaaaaccgttgacggtcaaactgaataatccgcgccaggaggtcagttttgtttacgataatcaactactgcatctcaaacagggcatttcagcctctggcgcgcgttacactgacggaatctatgttttctggtcgaaaggcgatgaagcgactgtctataaacgcgaccgcatcgtcttgaataactgtcagttacaaaatccacagcgttgagatttttccaggggcggcgcacaatagcgtcacccactgacaatccgtaaagaaaaccatgtctgataacgacgaattgcagcaaatcgcgcatctgcgccgtgaatacaccaaaggcgggttacgccgccgcgatcttcccgccgatccattaaccctttttgaacgttggctctctcaggcttgtgaagccaaactggcggaccctaccgcgatggtggtcgctaccgtggatgaacatggtcagccttatcagcgcatcgttttactcaaacattacgacgaaaaaggcatggtgttttacaccaacctcggcagccgtaaagcacatcaaatcgaaaataatccgcgcgttagcctgctgttcccgtggcatacccttgagcgccaggtgatggtgatcggtaaagcagaacgactttcgactctcgaagtgatgaaatattttcatagccgcccgcgtgatagccagattggtgcatgggtttcgaagcagtccagtcgcatttctgcccgcggtatccttgaaagtaaattcctggagctgaagcagaagtttcaacagggcgaagtgccattgccgagcttttggggcggttttcgcgtcagccttgaacagattgagttctggcagggtggtgagcatcgcctgcatgaccgctttttgtaccagcgtgaaaatgatgcgtggaagattgatcgtcttgcaccctgaaaagatgcaaaaatcttgctttaatcgctggtactcctgattctggcactttattctatgtctctttcgcatctggcgaaaagtcgtgtaccggcaaaggtgcagtcgttatatacatggagattttgatggcaagcagtaacttgattaaacaattgcaagagcgggggctggtatga

SEQ ID NO: 82 - protein, 218 aa, Escherichia coli
MSDNDELQQIAHLRREYTKGGLRRRDLPADPLTLFERWLSQACEAKLADPTAMVVATVDEHGQPYQRIVLLKHYDEKGMVFYTNLGSRKAHQIENNPRVSLLFPWHTLERQVMVIGKAERLSTLEVMKYFHSRPRDSQIGAWVSKQSSRISARGILESKFLELKQKFQQGEVPLPSFWGGFRVSLEQIEFWQGGEHRLHDRFLYQRENDAWKIDRLAP

SEQ ID NO: 83 - DNA, 852 bp, Escherichia coli
  CDS (1)..(852): pyridoxine /pyridoxal /pyridoxamine kinase (pdxK)
atgagcagcctgctgctgttcaacgacaagagcagggccctgcaggccgacatcgtggccgtgcagagccaggtggtgtacggcagcgtgggcaacagcatcgccgtgccggccatcaagcagaacggcctgaacgtgttcgccgtgccgaccgtgctgctgagcaacaccccgcactacgacaccttctacggcggcgccatcccggacgagtggttcagcggctacctgagggccctgcaggagagggacgccctgaggcagctgagggccgtgaccaccggctacatgggcaccgccagccagatcaagatcctggccgagtggctgaccgccctgaggaaggaccacccggacctgctgatcatggtggacccggtgatcggcgacatcgacagcggcatctacgtgaagccggacctgccggaggcctacaggcagtacctgctgccgctggcccagggcatcaccccgaacatcttcgagctggagatcctgaccggcaagaactgcagggacctggacagcgccatcgccgccgccaagagcctgctgagcgacaccctgaagtgggtggtgatcaccagcgccagcggcaacgaggagaaccaggagatgcaggtggtggtggtgagcgccgacagcgtgaacgtgatcagccacagcagggtgaagaccgacctgaagggcaccggcgacctgttctgcgcccagctgatcagcggcctgctgaagggcaaggccctgaacgacgccgtgcacagggccggcctgagggtgctggaggtgatgaggtacacccagcagcacgagagcgacgagctgatcctgccgccgctggccgaggcctga

SEQ ID NO: 84 - protein, 283 aa, Escherichia coli
MSSLLLFNDKSRALQADIVAVQSQVVYGSVGNSIAVPAIKQNGLNVFAVPTVLLSNTPHYDTFYGGAIPDEWFSGYLRALQERDALRQLRAVTTGYMGTASQIKILAEWLTALRKDHPDLLIMVDPVIGDIDSGIYVKPDLPEAYRQYLLPLAQGITPNIFELEILTGKNCRDLDSAIAAAKSLLSDTLKWVVITSASGNEENQEMQVVVVSADSVNVISHSRVKTDLKGTGDLFCAQLISGLLKGKALNDAVHRAGLRVLEVMRYTQQHESDELILPPLAEA

SEQ ID NO: 85 - DNA, 903 bp, Trypanosoma brucei
  CDS (1)..(903): pyridoxine /pyridoxal /pyridoxamine kinase (pdxK)
atgagcgagaagaccgtgctgagcatccagagcttcgtgacccacggctacgtgggcaacaaggccgccaccttcccgctgcagctgcacggcttcgacgtggacggcatcaacaccgtgtgcctgagcaaccacagcggctacccggtgatcaggggccacaggatgagcctgcaggagtacgacgagctgatggagggcgtgagggccaacaacttcctgagcaactacaggtacatcctgaccggctacatcaacaacgtggacatcatcggcaggatcagggacaccctgaaggaggtgagggagctgagggagaaggaggacaagaagctgaccttcatctgcgacccggtgatgggcgacgacggcatcatgtactgcaagaaggaggtgctggacgcctacagggagctggtgccgctggccgacatcgtgaccccgaactacttcgaggccagcctgctgagcggcgtgaccgtgaacgacctgagcagcgccatcctggccgccgactggttccacaactgcggcgtggcccacgtgatcatcaagagcttcagggagcaggagaacccgacccacctgaggttcctgtacagcgtgaaggagggcagcgaggccgccgtgaggaggttcagcggcgtggtgccgtaccacgagggcaggtacaccggcaccggcgacgtgttcgccgcctgcctgctggccttcagccacagccacccgatggacgtggccatcggcaagagcatggccgtgctgcaggagctgatcatcgccaccaggaaggagggcggcgacggcaagagcagcctgaagagcagggagctgagggtggtggccagcccgcaggtggtgctgcagccgagcaccgtggtggacgtgaagccgatcagctga

SEQ ID NO: 86 - protein, 300 aa, Trypanosoma brucei
MSEKTVLSIQSFVTHGYVGNKAATFPLQLHGFDVDGINTVCLSNHSGYPVIRGHRMSLQEYDELMEGVRANNFLSNYRYILTGYINNVDIIGRIRDTLKEVRELREKEDKKLTFICDPVMGDDGIMYCKKEVLDAYRELVPLADIVTPNYFEASLLSGVTVNDLSSAILAADWFHNCGVAHVIIKSFREQENPTHLRFLYSVKEGSEAAVRRFSGVVPYHEGRYTGTGDVFAACLLAFSHSHPMDVAIGKSMAVLQELIIATRKEGGDGKSSLKSRELRVVASPQVVLQPSTVVDVKPIS

SEQ ID NO: 87 - DNA, 966 bp, Caenorhabditis elegans
  CDS (1)..(966): pyridoxine /pyridoxal /pyridoxamine kinase (pdxK)
atgagcagcagcgagctgatcgccgagctggagagggagagggacaggagggtgctgagcatccagagccacgtggtgcacggctacgccggcaacaagtgcagcgtgttcccgctgcagctgcacggcttcgaggtggacttcatcaacagcgtgcagttcagcaaccacgccggcaacatcgagtacctgaccctgccgaccaggtacgagcacgtgaagggccagaagctgaccgagaaggagctggaggagctgtacgagggcctgaccctgaacaacatcaacaactacacccacgtgctgaccggctactgcggcaacgtgaccttcctgcagaagatcgccgacgtggtgaaggacctgaagaagaagaacggcaacaccaccttcgtgtgcgacccggtgatgggcgacaacggcaggtactacaccccgaaggagctgatgccggtgtacagggacctgatcatcccgctggccgacgtgctgaccccgaacgccttcgagctgggcgagctgaccggcagcccgatcgagaccgaggaggactgcctgagggccgtgaacgagctgcacgccaagggcgtgaagaccgtggtggtgaccagcggcgtgaccggcgcccagaccaacgagagcctgaggtgctacgccagcgtgaagggcagccacgtgtacaggttcaccttcccgaggctggtgggccagttcgtgggcaccggcgacaccttcaccagcctgctggtggtgtggctggacgagctgaacggcgacgtgagcgaggccgtgaagagggtgctggccagcatgcagtgcctgatcaggaagaccagcagctacgcccagctgcaggtggacaccaacagcagggccatgtgcgagctgaggctgatccagagcaggaaggacctgctgtggccgccgacctgcgaccagatccaggtggagaagatcggccagtga

SEQ ID NO: 88 - protein, 321 aa, Caenorhabditis elegans
MSSSELIAELERERDRRVLSIQSHVVHGYAGNKCSVFPLQLHGFEVDFINSVQFSNHAGNIEYLTLPTRYEHVKGQKLTEKELEELYEGLTLNNINNYTHVLTGYCGNVTFLQKIADVVKDLKKKNGNTTFVCDPVMGDNGRYYTPKELMPVYRDLIIPLADVLTPNAFELGELTGSPIETEEDCLRAVNELHAKGVKTVVVTSGVTGAQTNESLRCYASVKGSHVYRFTFPRLVGQFVGTGDTFTSLLVVWLDELNGDVSEAVKRVLASMQCLIRKTSSYAQLQVDTNSRAMCELRLIQSRKDLLWPPTCDQIQVEKIGQ

SEQ ID NO: 89 - DNA, 867 bp, Salmonella typhimurium
  CDS (1)..(867): pyridoxal kinase (yfei)
atgggccaggagagcgacatccagagcgtgctgttcgacgacaaccacagggccctgcagaccgacatcgtggccgtgcagagccaggtggtgtacggcagcgtgggcaacagcatcgccgtgccggccatcaaggcccagggcctgagggtgaccgccgtgccgaccgtgctgttcagcaacaccccgcactacaagaccttctacggcggcatcatcccggccgagtggttcgccggctacctgaccgccctgaacgagagggacgccctgagggagctgaaggccatcaccaccggctacatgggcagcgccgaccagatcgtgctgctgagcaagtggctgatggccatcagggccagccacccggaggtgtgcatcctggtggacccggtgatcggcgacaccgacagcggcatgtacgtgcaggccgagatcccgcaggcctacaggacccacctgctgccgcaggcccagggcctgaccccgaacgtgttcgagctggagatgctgagcggcaagccgtgcaggaccctggaggaggccgtggccgccgcccagagcctgctgagcgacaccctgaagtgggtggtgatcaccagcgccccgggcgagagcctggagaccatcaccgtggccgtggtgaccgcccaggtggtggaggtgttcgcccacccgagggtggccaccgagctgaagggcaccggcgacctgttctgcgccgagctggtgagcggcatcgtgcagggcaagaagctgaccaccgccgccaaggacgccgcccagagggtgctggaggtgatgacctggacccagcagtgcggctgcgacgagctgatcctgccgccggccggcgaggccaggtga

SEQ ID NO: 90 - protein, 288 aa, Salmonella typhimurium [sequence omitted]

SEQ ID NO: 91 - DNA, 633 bp, Haemophilus influenzae
  CDS (1)..(633): pdxH (yfei)
atggagctgcacaacatcagggacgagtacaccaagagggtgctgagccagcacgactgccacaagaacccgatcagccagttcgagcagtggcagaaggaggccatccacgcccaggtgaacgagccgaccgccatgaacatcgccaccgtggacgagcagggcaggccgaacagcaggatggtgctgctgaaggaggtgaacgagcagggcttcgtgttcttcaccaactacctgagcaggaagggcggctgcatcgagaggaacccgtacgtggccctgaccttcttctggccggagctggagaggcaggtgaggatcgagggcaaggccgtgaagatcccggccgagcagagcgacaagtacttcgccaccaggccgtacaccagcaggatcggcgcctgggccagcgagcagagcgccgtgatcagcaactacaagagcctgctggccaaggccgccctggtggccgccaagcacccgctgaacgtgccgaggccggactactggggcggctacctggtggtgccggagaccgtggagttctggcagggcaggccgagcaggctgcacgacaggatcaggtacaggaaggagagcgacaactggatcagggagaggctgagcccgtga

SEQ ID NO: 92 - protein, 210 aa, Haemophilus influenzae
MELHNIRDEYTKRVLSQHDCHKNPISQFEQWQKEAIHAQVNEPTAMNIATVDEQGRPNSRMVLLKEVNEQGFVFFTNYLSRKGGCIERNPYVALTFFWPELERQVRIEGKAVKIPAEQSDKYFATRPYTSRIGAWASEQSAVISNYKSLLAKAALVAAKHPLNVPRPDYWGGYLVVPETVEFWQGRPSRLHDRIRYRKESDNWIRERLSP

SEQ ID NO: 93 - DNA, 939 bp, Saccharomyces cerevisiae
  CDS (1)..(939): (Yn8fp)
atgccgaggctgctggccacccagagccacgtggtgcacggctacgtgggcaacaaggccgccaccttcccgctgcagtgcctgggctgggacgtggactgctgcaacagcgtgcagttcagcaaccacaccggctacggcctggacaaggtgttcggcaccatcaccagggagaccgacctgaaggagctgctgagcggcctgttcgacaacttcagccaggactaccaggccctgctgagcggctacctgccgaacaagaacagcgtgaggtgcatgggcacctactacgccaagttcaaggaggccaacccggagatgatctggctgatggacccggtgatgggcgacgagggccagctgtacgtgagcgaggacgtgatcccggagtacaggaagctggccctgagcccgaagcagctggtggacatcatcaccccgaaccagttcgagctggagatcctgtacggcggcgagatcaagaccaaggagcacctgaagaaggccctgaagaagctgcaccagaccatcccggtgatcatcgtgaccagctgcgactgcaagatgttcgacgacaaggacttcatctactgcgtggccagcatggagggcaagaccccgatcgtgtacagggtgccgttcatcgacagctacttcaccggcgtgggcgacctgttcagcgccctgctgctggacagggtgtacaagatcctgagcaacccgaccaccaccctgaagttcgaggaccaggtgaacaacgtgctgaacgtgatccagaaggtgctgaagatcaccaggagctacgccagcggcaagatgaaggccaagatgggcagcgccctggagatgaaggagatggagctgaggctgatcgagagcagggacatctacgagaccatcaacatccaccagaccgactacatctacgccaggctgtag

SEQ ID NO: 94 - protein, 312 aa, Saccharomyces cerevisiae
MPRLLATQSHVVHGYVGNKAATFPLQCLGWDVDCCNSVQFSNHTGYGLDKVFGTITRETDLKELLSGLFDNFSQDYQALLSGYLPNKNSVRCMGTYYAKFKEANPEMIWLMDPVMGDEGQLYVSEDVIPEYRKLALSPKQLVDIITPNQFELEILYGGEIKTKEHLKKALKKLHQTIPVIIVTSCDCKMFDDKDFIYCVASMEGKTPIVYRVPFIDSYFTGVGDLFSALLLDRVYKILSNPTTTLKFEDQVNNVLNVIQKVLKITRSYASGKMKAKMGSALEMKEMELRLIESRDIYETINIHQTDYIYARL

SEQ ID NO: 95 - DNA, 666 bp, Synechocystis sp. PCC 6803
  CDS (1)..(666): pdxJ (SLR11779)
atggagccagacccgatcgccgccgctgtgttggccgaactggccggggcggatggtattacagcccatttacgggaagaccgtcgccacattcaagagcgggacgtggaaatattgcgaaaaacggtgcgcacccacttaaacctggagatggccgccaccaaggaaatggtggaaattgccctcaaccttaaaccggattacgtcaccctggtaccggaacggcgggaagaagtgaccacggaagggggtttggatgtggctgggaacttgaactatttgctgggagtagttgagcagttacaatcccgacatattcccgttagtcttttcatcgacccggatgtggcccaactgaaagcctctgcccaaaccggagcaaaattcattgaattacatacgggcaaatatgccaatgcccccaccgctgacgaccaagctagggaattggggtccctggcgatcgcctgtgacattgctctagaactggggctaaggatcaacgctggccatgggttgacctattggaatgtgcgcccagtggcggaattaccgggcatggaagagttaaacatcggccacagcatcatgagccgggccattctggtgggcatggaaagggccgtgcgggaaatgaaattagccatgttggggctgccattttaa

SEQ ID NO: 96 - protein, 221 aa, Synechocystis sp. PCC 6803
MEPDPIAAAVLAELAGADGITAHLREDRRHIQERDVEILRKTVRTHLNLEMAATKEMVEIALNLKPDYVTLVPERREEVTTEGGLDVAGNLNYLLGVVEQLQSRHIPVSLFIDPDVAQLKASAQTGAKFIELHTGKYANAPTADDQARELGSLAIACDIALELGLRINAGHGLTYWNVRPVAELPGMEELNIGHSIMSRAILVGMERAVREMKLAMLGLPF

SEQ ID NO: 97 - DNA, 732 bp, Escherichia coli
  CDS (1)..(732): PDXJ
atggccgagctgctgctgggcgtgaacatcgaccacatcgccaccctgaggaacgccaggggcaccgcctacccggacccggtgcaggccgccttcatcgccgagcaggccggcgccgacggcatcaccgtgcacctgagggaggacaggaggcacatcaccgacagggacgtgaggatcctgaggcagaccctggacaccaggatgaacctggagatggccgtgaccgaggagatgctggccatcgccgtggagaccaagccgcacttctgctgcctggtgccggagaagaggcaggaggtgaccaccgagggcggcctggacgtggccggccagagggacaagatgagggacgcctgcaagaggctggccgacgccggcatccaggtgagcctgttcatcgacgccgacgaggagcagatcaaggccgccgccgaggtgggcgccccgttcatcgagatccacaccggctgctacgccgacgccaagaccgacgccgagcaggcccaggagctggccaggatcgccaaggccgccaccttcgccgccagcctgggcctgaaggtgaacgccggccacggcctgacctaccacaacgtgaaggccatcgccgccatcccggagatgcacgagctgaacatcggccacgccatcatcggcagggccgtgatgaccggcctgaaggacgccgtggccgagatgaagaggctgatgctggaggccaggggctga

SEQ ID NO: 98 - protein, 243 aa, Escherichia coli
MAELLLGVNIDHIATLRNARGTAYPDPVQAAFIAEQAGADGITVHLREDRRHITDRDVRILRQTLDTRMNLEMAVTEEMLAIAVETKPHFCCLVPEKRQEVTTEGGLDVAGQRDKMRDACKRLADAGIQVSLFIDADEEQIKAAAEVGAPFIEIHTGCYADAKTDAEQAQELARIAKAATFAASLGLKVNAGHGLTYHNVKAIAAIPEMHELNIGHAIIGRAVMTGLKDAVAEMKRLMLEARG

SEQ ID NO: 99 - DNA, 930 bp, Arabidopsis thaliana
  CDS (1)..(930): PDX1.1
atggccggcaccggcgtggtggccgtgtacggcgagggcgccatgaccgagaccaagcagaagagcccgttcagcgtgaaggtgggcctggcccagatgctgaggggcggcgtgatcatggacgtggtgaacgccgagcaggccaggatcgccgaggaggccggcgcctgcgccgtgatggccctggagagggtgccggccgacatcagggcccagggcggcgtggccaggatgagcgacccggagatgatcaaggagatcaagaacgccgtgaccatcccggtgatggccaaggccaggatcggccacttcgtggaggcccagatcctggaggccatcggcgtggactacgtggacgagagcgaggtgctgaccctggccgacgaggacaaccacatcaacaagcacaacttcaagatcccgttcgtgtgcggctgcaggaacctgggcgaggccctgaggaggatcagggagggcgccgccatgatcaggaccaagggcgaggccggcaccggcaacgtggtggaggccgtgaggcacgtgaggagcgtgaacggcgccatcaggctgctgaggagcatggacgacgacgaggtgttcacctacgccaagaagatcgccgccccgtacgacctggtggtgcagaccaaggagctgggcaggctgccggtggtgcagttcgccgccggcggcgtggccaccccggccgacgccgccctgatgatgcagctgggctgcgacggcgtgttcgtgggcagcggcgtgttcaagagcggcgacccggtgaagagggccaaggccatcgtgcaggccgtgaccaactacagggacgccgccgtgctggccgaggtgagctgcggcctgggcgaggccatggtgggcctgaacctggacgacaaggtggagaggttcgccagcaggagcgagtga

SEQ ID NO: 100 - protein, 309 aa, Arabidopsis thaliana
MAGTGVVAVYGEGAMTETKQKSPFSVKVGLAQMLRGGVIMDVVNAEQARIAEEAGACAVMALERVPADIRAQGGVARMSDPEMIKEIKNAVTIPVMAKARIGHFVEAQILEAIGVDYVDESEVLTLADEDNHINKHNFKIPFVCGCRNLGEALRRIREGAAMIRTKGEAGTGNVVEAVRHVRSVNGAIRLLRSMDDDEVFTYAKKIAAPYDLVVQTKELGRLPVVQFAAGGVATPADAALMMQLGCDGVFVGSGVFKSGDPVKRAKAIVQAVTNYRDAAVLAEVSCGLGEAMVGLNLDDKVERFASRSE

SEQ ID NO: 101 - DNA, 930 bp, Arabidopsis thaliana
  CDS (1)..(930): PDX1.3
atggagggcaccggcgtggtggccgtgtacggcaacggcgccatcaccgaggccaagaagagcccgttcagcgtgaaggtgggcctggcccagatgctgaggggcggcgtgatcatggacgtggtgaacgccgagcaggccaggatcgccgaggaggccggcgcctgcgccgtgatggccctggagagggtgccggccgacatcagggcccagggcggcgtggccaggatgagcgacccgcagatgatcaaggagatcaagcaggccgtgaccatcccggtgatggccaaggccaggatcggccacttcgtggaggcccagatcctggaggccatcggcatcgactacatcgacgagagcgaggtgctgaccctggccgacgaggaccaccacatcaacaagcacaacttcaggatcccgttcgtgtgcggctgcaggaacctgggcgaggccctgaggaggatcagggagggcgccgccatgatcaggaccaagggcgaggccggcaccggcaacatcatcgaggccgtgaggcacgtgaggagcgtgaacggcgacatcagggtgctgaggaacatggacgacgacgaggtgttcaccttcgccaagaagctggccgccccgtacgacctggtgatgcagaccaagcagctgggcaggctgccggtggtgcagttcgccgccggcggcgtggccaccccggccgacgccgccctgatgatgcagctgggctgcgacggcgtgttcgtgggcagcggcatcttcaagagcggcgacccggccaggagggccagggccatcgtgcaggccgtgacccactacagcgacccggagatgctggtggaggtgagctgcggcctgggcgaggccatggtgggcatcaacctgaacgacgagaaggtggagaggttcgccaacaggagcgagtga

SEQ ID NO: 102 - protein, 309 aa, Arabidopsis thaliana
MEGTGVVAVYGNGAITEAKKSPFSVKVGLAQMLRGGVIMDVVNAEQARIAEEAGACAVMALERVPADIRAQGGVARMSDPQMIKEIKQAVTIPVMAKARIGHFVEAQILEAIGIDYIDESEVLTLADEDHHINKHNFRIPFVCGCRNLGEALRRIREGAAMIRTKGEAGTGNIIEAVRHVRSVNGDIRVLRNMDDDEVFTFAKKLAAPYDLVMQTKQLGRLPVVQFAAGGVATPADAALMMQLGCDGVFVGSGIFKSGDPARRARAIVQAVTHYSDPEMLVEVSCGLGEAMVGINLNDEKVERFANRSE

SEQ ID NO: 103 - DNA, 1032 bp, Cercospora nicotianae
  CDS (1)..(1032): PDX1
atggccagcaacggcaccagcgtgagcccgttcaggagccagaagaacgccgccatggccgtgaacgacaccccggccaacggccacgccgagccgagcaccatcaccgccgccagcaagaccaacaccaccaagatcaccagccagaacgacccgcagagcagcttcgccgtgaaggtgggcctggcccagatgctgaagggcggcgtgatcatggacgtggtgaacgccgagcaggccaggatcgccgaggaggccggcgcctgcgccgtgatggccctggagagggtgccggccgacatcaggaaggacggcggcgtggccaggatgagcgacccgcagatgatcaaggacatcatgaacgccgtgaccatcccggtgatggccaagagcaggatcggccacttcgtggagtgccagatcctgcaggccatcggcgtggactacatcgacgagagcgaggtgctgaccccggccgacccggtgaaccacatcgacaagagcgtgtacaacgtgccgttcgtgtgcggctgcaagaacctgggcgaggccctgaggaggatcagcgagggcgccgccatgatcaggaccaagggcgaggccggcaccggcgacgtggtggaggccgtgaggcacatgcagaccgtgaacgccgagatcgccaaggccagcagcgccagcgacgccgacctgaggatgatggccagggagctgcagtgcgactacaacctgctgaagcagaccgcccagctgaagaggctgccggtggtgaacttcgccgccggcggcatcgccaccccggccgacgccgccctgatgatgcagatgggctgcgacggcgtgttcgtgggcagcggcatcttcaagagcggcgacgccgccaagagggccaaggccatcgtgcaggccaccacccactacaacgacccgaaggtgctggccgaggtgagcagcggcctgggcgaggccatggtgggcatcaactgcgacaagctgccggagacccagaagctggccaccaggggctggtga

SEQ ID NO: 104 - protein, 343 aa, Cercospora nicotianae
MASNGTSVSPFRSQKNAAMAVNDTPANGHAEPSTITAASKTNTTKITSQNDPQSSFAVKVGLAQMLKGGVIMDVVNAEQARIAEEAGACAVMALERVPADIRKDGGVARMSDPQMIKDIMNAVTIPVMAKSRIGHFVECQILQAIGVDYIDESEVLTPADPVNHIDKSVYNVPFVCGCKNLGEALRRISEGAAMIRTKGEAGTGDVVEAVRHMQTVNAEIAKASSASDADLRMMARELQCDYNLLKQTAQLKRLPVVNFAAGGIATPADAALMMQMGCDGVFVGSGIFKSGDAAKRAKAIVQATTHYNDPKVLAEVSSGLGEAMVGINCDKLPETQKLATRGW

SEQ ID NO: 105 - DNA, 1233 bp, Saccharomyces cerevisiae
  CDS (1)..(1233): PDX1
atgctgagcgccatcagcaaggtgagcaccctgaagagctgcaccaggtacctgaccaagtgcaactaccacgccagcgccaagctgctggccgtgaagaccttcagcatgccggccatgagcccgaccatggagaagggcggcatcgtgagctggaagtacaaggtgggcgagccgttcagcgccggcgacgtgatcctggaggtggagaccgacaagagccagatcgacgtggaggccctggacgacggcaagctggccaagatcctgaaggacgagggcagcaaggacgtggacgtgggcgagccgatcgcctacatcgccgacgtggacgacgacctggccaccatcaagctgccgcaggaggccaacaccgccaacgccaagagcatcgagatcaagaagccgagcgccgacagcaccgaggccacccagcagcacctgaagaaggccaccgtgaccccgatcaagaccgtggacggcagccaggccaacctggagcagaccctgctgccgagcgtgagcctgctgctggccgagaacaacatcagcaagcagaaggccctgaaggagatcgccccgagcggcagcaacggcaggctgctgaagggcgacgtgctggcctacctgggcaagatcccgcaggacagcgtgaacaaggtgaccgagttcatcaagaagaacgagaggctggacctgagcaacatcaagccgatccagctgaagccgaagatcgccgagcaggcccagaccaaggccgccgacaagccgaagatcaccccggtggagttcgaggagcagctggtgttccacgccccggccagcatcccgttcgacaagctgagcgagagcctgaacagcttcatgaaggaggcctaccagttcagccacggcaccccgctgatggacaccaacagcaagtacttcgacccgatcttcgaggacctggtgaccctgagcccgagggagccgaggttcaagttcagctacgacctgatgcagatcccgaaggccaacaacatgcaggacacctacggccaggaggacatcttcgacctgctgaccggcagcgacgccaccgccagcagcgtgaggccggtggagaagaacctgccggagaagaacgagtacatcctggccctgaacgtgagcgtgaacaacaagaagttcaacgacgccgaggccaaggccaagaggttcctggactacgtgagggagctggagagcttctga

SEQ ID NO: 106 - protein, 410 aa, Saccharomyces cerevisiae
MLSAISKVSTLKSCTRYLTKCNYHASAKLLAVKTFSMPAMSPTMEKGGIVSWKYKVGEPFSAGDVILEVETDKSQIDVEALDDGKLAKILKDEGSKDVDVGEPIAYIADVDDDLATIKLPQEANTANAKSIEIKKPSADSTEATQQHLKKATVTPIKTVDGSQANLEQTLLPSVSLLLAENNISKQKALKEIAPSGSNGRLLKGDVLAYLGKIPQDSVNKVTEFIKKNERLDLSNIKPIQLKPKIAEQAQTKAADKPKITPVEFEEQLVFHAPASIPFDKLSESLNSFMKEAYQFSHGTPLMDTNSKYFDPIFEDLVTLSPREPRFKFSYDLMQIPKANNMQDTYGQEDIFDLLTGSDATASSVRPVEKNLPEKNEYILALNVSVNNKKFNDAEAKAKRFLDYVRELESF

SEQ ID NO: 107 - DNA, 918 bp, Bacillus subtilis
  CDS (1)..(918): PDX1
atggccagcatggcccagaccggcaccgagagggtgaagaggggcatggccgagatgcagaagggcggcgtgatcatggacgtgatcaacgccgagcaggccaagatcgccgaggaggccggcgccgtggccgtgatggccctggagagggtgccggccgacatcagggccgccggcggcgtggccaggatggccgacccgaccatcgtggaggaggtgatgaacgccgtgagcatcccggtgatggccaaggccaggatcggccacatcgtggaggccagggtgctggaggccatgggcgtggactacatcgacgagagcgaggtgctgaccccggccgacgaggagttccacctgaacaagaacgagtacaccgtgccgttcgtgtgcggctgcagggacctgggcgaggccaccaggaggatcgccgagggcgccagcatgctgaggaccaagggcgagccgggcaccggcaacatcgtggaggccgtgaggcacatgaggaaggtgaacgcccaggtgaggaaggtggtggccatgagcgaggacgagctgatgaccgaggccaagaacctgggcgccccgtacgagctgctgctgcagatcaagaaggacggcaagctgccggtggtgaacttcgccgccggcggcgtggccaccccggccgacgccgccctgatgatgcagctgggcgccgacggcgtgttcgtgggcagcggcatcttcaagagcgacaacccggccaagttcgccaaggccatcgtggaggccaccacccacttcaccgactacaagctgatcgccgagctgagcaaggagctgggcaccgccatgaagggcatcgagatcagcaacctgctgccggagcagaggatgcaggagaggggctggctggagcaccaccaccaccaccactga

SEQ ID NO: 108 - protein, 305 aa, Bacillus subtilis
MASMAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWLEHHHHHH

SEQ ID NO: 109 - DNA, 957 bp, Oryza sativa
  CDS (1)..(957): PDX1.1
atggccaccgacggcaccggcgtggtgaccgtgtacggcagcggcaccaacggcgccgccctgctggagccgagcaaccacaagagcgccaccttcagcgtgaaggtgggcctggcccagatgctgaggggcggcgtgatcatggacgtggtgaccccggagcaggccaggatcgccgaggaggccggcgcctgcgccgtgatggccctggagagggtgccggccgacatcagggcccagggcggcgtggccaggatgagcgacccgggcctgatcagggacatcaagagggccgtgaccatcccggtgatggccaaggccaggatcggccacttcgtggaggcccagatcctggaggccatcggcgtggactacgtggacgagagcgaggtgctgaccctggccgacgacgcccaccacatcaacaagcacaacttcagggtgccgttcgtgtgcggctgcagggacctgggcgaggccctgaggaggatcagggagggcgccgccatgatcaggaccaagggcgaggccggcaccggcaacgtggtggaggccgtgaggcacgtgaggagcgtgatgggcgacatcagggccctgaggaacatggacgacgacgaggtgttcagctacgccaagaggatcgccgccccgtacgacctggtgatgcagaccaagcagctgggcaggctgccggtggtgcagttcgccgccggcggcgtggccaccccggccgacgccgccctgatgatgcagctgggctgcgacggcgtgttcgtgggcagcggcatcttcaagagcggcgacccggccaggagggccagggccatcgtgcaggccgtgacccactacagcgacccgaagatcctggccgaggtgagcagcggcctgggcgaggccatggtgggcatcaacctgagcgacccgaaggtggagaggttcgccgccaggagcgagtga

SEQ ID NO: 110 - protein, 318 aa, Oryza sativa
MATDGTGVVTVYGSGTNGAALLEPSNHKSATFSVKVGLAQMLRGGVIMDVVTPEQARIAEEAGACAVMALERVPADIRAQGGVARMSDPGLIRDIKRAVTIPVMAKARIGHFVEAQILEAIGVDYVDESEVLTLADDAHHINKHNFRVPFVCGCRDLGEALRRIREGAAMIRTKGEAGTGNVVEAVRHVRSVMGDIRALRNMDDDEVFSYAKRIAAPYDLVMQTKQLGRLPVVQFAAGGVATPADAALMMQLGCDGVFVGSGIFKSGDPARRARAIVQAVTHYSDPKILAEVSSGLGEAMVGINLSDPKVERFAARSE

SEQ ID NO: 111 - DNA, 930 bp, Hevea brasilensis
  CDS (1)..(930): PDX1
atggccggcaccggcgtggtggccgtgtacggcaacggcgccatcaccgagaccaagaagagcccgttcagcgtgaaggtgggcctggcccagatgctgaggggcggcgtgatcatggacgtggtgaacccggagcaggccaggatcgccgaggaggccggcgcctgcgccgtgatggccctggagagggtgccggccgacatcagggcccagggcggcgtggccaggatgagcgacccgcagctgatcaaggagatcaagcagagcgtgaccatcccggtgatggccaaggccaggatcggccacttcgtggaggcccagatcctggaggccatcggcatcgactacgtggacgagagcgaggtgctgaccccggccgacgaggagaaccacatcaacaagcacaacttcaggatcccgttcgtgtgcggctgcaggaacctgggcgaggccctgaggaggatcagggagggcgccgccatgatcaggaccaagggcgaggccggcaccggcaacgtgatcgaggccgtgaggcacgtgaggagcgtgatgggcgacatcaggctgctgaggaacatggacgacgacgaggtgttcaccttcgccaagaagatcgccgccccgtacgacctggtgatgcagaccaagcagctgggcaggctgccggtggtgcagttcgccgccggcggcgtggccaccccggccgacgccgccctgatgatgcagctgggctgcgacggcgtgttcgtgggcagcggcgtgttcaagagcggcgacccggccaggagggccagggccatcgtgcaggccgtgacccactacagcgacccggacatgctggccgaggtgagctgcggcctgggcgaggccatggtgggcatcaacctgaacgacaagaaggtggagaggttcgccaacaggagcgagtga

SEQ ID NO: 112 - protein, 309 aa, Hevea brasilensis
MAGTGVVAVYGNGAITETKKSPFSVKVGLAQMLRGGVIMDVVNPEQARIAEEAGACAVMALERVPADIRAQGGVARMSDPQLIKEIKQSVTIPVMAKARIGHFVEAQILEAIGIDYVDESEVLTPADEENHINKHNFRIPFVCGCRNLGEALRRIREGAAMIRTKGEAGTGNVIEAVRHVRSVMGDIRLLRNMDDDEVFTFAKKIAAPYDLVMQTKQLGRLPVVQFAAGGVATPADAALMMQLGCDGVFVGSGVFKSGDPARRARAIVQAVTHYSDPDMLAEVSCGLGEAMVGINLNDKKVERFANRSE

SEQ ID NO: 113 - DNA, 669 bp, Stellaria longipes
  CDS (1)..(669): PDX1
atggccaaggccaggatcggccacttcgtggaggcccagatcctggagagcatcggcgtggactacgtggacgagagcgaggtgctgaccccggccgacgaggaccaccacatcaacaagcacaacttccagatcccgttcgtgtgcggcctgagcatcccgaggggcgccccgccgccggcctacaggaggggctacgccggccacgacaccggcccgagggtgaggaagccgagcaccggcaacgtggtggaggccgtgaggcacatcaggagcgtgatgggcgagatcaggctgctgaggaacatggacgacgacgaggtgttcgcctacgccaagaagatcagcgccgcctacgacctggtgatgcagaccaagcagctgggcaggctgccggtggtgaacttcgccgccggcggcgtggccaccccggccgacgccgccctgatgatgcagctgggctgcgacggcgtgttcgtgggcagcggcgtgttcaagagcggcgacccggccaagagggccagggccatcgtgcaggccgtgacccactacagcgacccggacctgctgggcaggggcgagttcggcctgggcgaggccatggtgggcatcatcgtgaggatgaggaggctgaggggcaccccgatcgtgctgaacgaggtgatctga

SEQ ID NO: 114 - protein, 222 aa, Stellaria longipes
MAKARIGHFVEAQILESIGVDYVDESEVLTPADEDHHINKHNFQIPFVCGLSIPRGAPPPAYRRGYAGHDTGPRVRKPSTGNVVEAVRHIRSVMGEIRLLRNMDDDEVFAYAKKISAAYDLVMQTKQLGRLPVVNFAAGGVATPADAALMMQLGCDGVFVGSGVFKSGDPAKRARAIVQAVTHYSDPDLLGRGEFGLGEAMVGIIVRMRRLRGTPIVLNEVI

SEQ ID NO: 115 - DNA, 933 bp, Brassica napus
  CDS (1)..(933): PDX1
atggcggaatcagccggagtggtggcggtttacggcgaaggatccatgaccgagacgaagcagcaatctcccttctccgtcaaggtcggcctcgctcagatgctccgcggcggcgtgatcatggacgtcgtcaacgccgagcaagcgcgaatcgccgaagaagccggcgcctgcgccgtcatggctctcgagcgcgttcccgccgatatcagagcgcaaggtggcgtcgcgaggatgagcgatccggagatgatcaaagagatcaaacaggcggttacgattccggtcatggccaaagctcgaatcggccacttcgtcgaggcgcagatcctcgagtccatcggagtcgattacgtcgacgagagtgaagtcctcactctcgccgacgaggacaaccacatcaacaagcacaacttcaagataccgttcgtttgtggatgcaggaacctcggggaagccttgcggcggatccgcgaaggagccgcgatgataaggaccaaaggagaggccgggacggggaacgtcgtggaggccgttaggcacgtgaggagcgtgatgggagcgataaggttgctgagaggcatggatgacgacgaggtgttcgcttacgcgaagaagatcgctgcgccttatgagttggttatgcagacgaagcagctcgggaggttaccggtggttcagttcgccgccggaggggtcgcgacgccggcggacgcggcgctgatgatgcagctggggtgtgatggagtgttcgttgggtcgggtattttcaagagtggagatccggcgaagcgtgcgaaggcgattgtgcaggcggttacgaattatagagacgcggcggtgttggcggaggtgagctgtgggttgggtgaagctatggttggtatcaatttggatgataaggttgagaggttcgcgaaccggtctgagtga

SEQ ID NO: 116 - protein, 310 aa, Brassica napus
MAESAGVVAVYGEGSMTETKQQSPFSVKVGLAQMLRGGVIMDVVNAEQARIAEEAGACAVMALERVPADIRAQGGVARMSDPEMIKEIKQAVTIPVMAKARIGHFVEAQILESIGVDYVDESEVLTLADEDNHINKHNFKIPFVCGCRNLGEALRRIREGAAMIRTKGEAGTGNVVEAVRHVRSVMGAIRLLRGMDDDEVFAYAKKIAAPYELVMQTKQLGRLPVVQFAAGGVATPADAALMMQLGCDGVFVGSGIFKSGDPAKRAKAIVQAVTNYRDAAVLAEVSCGLGEAMVGINLDDKVERFANRSE

SEQ ID NO: 117 - DNA, 942 bp, Physcomitrella patens
  CDS (1)..(942): PDX1
atggaggagaacagggtggtggccgtgctgaacaacggcagcggcctgagcgagaaccagaacaagaagaccgtgagctacgccgtgaaggtgggcctggcccagatgctgaggggcggcgtgatcatggacgtggtggacgtggcccaggccaggatcgccgaggaggccggcgccgtggccgtgatggccctggagagggtgccggccgacatcagggccgagggcggcgtggccaggatgagcgacccgagcatgatcaaggagatcaagaaggccgtgaccatcccggtgatggccaaggccaggatcggccacttcgtggaggcccagatcctggaggccatcggcgtggactacatcgacgagagcgaggtgctgaccccggccgacgacgtgcaccacatcaacaagcacaactacaggatcccgttcgtgtgcggctgcaggaacctgggcgaggccctgaggaggatcgccgagggcgccgccatgatcaggaccaagggcgaggccggcaccggcaacgtggtggaggccgtgaggcacaccaggagcgtgctgggcgacatcaggaggctgcagagcctggacgacgacgaggtgttcacctacgccaaggagatccaggccccgtacgagctggtgaggcagaccaagcagctgggcaggctgccggtggtgaacttcgccgccggcggcgtggccaccccggccgacgccgccctgatgatgcagctgggctgcgacggcgtgttcgtgggcagcggcatcttcaagagcggcgacccggccaagagggccagggccatcgtggaggccgtgacccactacaacgacgccagggtgctggccgaggtgagcgagaacctgggcgaggccatggtgggcatcaacctgagcgacaagaaggtggagaggttcgccgccaggagcgagtga

SEQ ID NO: 118 - protein, 313 aa, Physcomitrella patens
MEENRVVAVLNNGSGLSENQNKKTVSYAVKVGLAQMLRGGVIMDVVDVAQARIAEEAGAVAVMALERVPADIRAEGGVARMSDPSMIKEIKKAVTIPVMAKARIGHFVEAQILEAIGVDYIDESEVLTPADDVHHINKHNYRIPFVCGCRNLGEALRRIAEGAAMIRTKGEAGTGNVVEAVRHTRSVLGDIRRLQSLDDDEVFTYAKEIQAPYELVRQTKQLGRLPVVNFAAGGVATPADAALMMQLGCDGVFVGSGIFKSGDPAKRARAIVEAVTHYNDARVLAEVSENLGEAMVGINLSDKKVERFAARSE

SEQ ID NO: 119 - DNA, 891 bp, Schizosaccharomyces pombe
  CDS (1)..(891): PDX1
atgagcgccgagatcaagggcagcacccaggtgaaggccggcctggcccagatgctgaagggcggcgtgatcatggacgtggtgaacgccgagcaggccaggatcgccgaggccgccggcgcctgcgccgtgatggccctggagagggtgccggccgacatcagggcccagggcggcgtggccaggatgagcgacccgagcatgatcaaggagatccaggccgccgtgagcatcccggtgatggccaaggtgaggatcggccacttcgtggaggcccagatcctggagagcatcggcgtggactacatcgacgagagcgaggtgctgaccccggccgacgacatcaaccacatcgagaagagcaagttcaccgtgccgttcgtgtgcggcagcaggaacctgggcgaggccctgaggaggatcagcgagggcgccgccatgatcaggaccaagggcgaggccggcaccggcgacgtggtggaggccgtgaggcacaccaggcagatgcagggcgagctgaggagggtgaagagcatgagcccggacgagctgtacacctacgccaaggagatcgccgccccgatcgacctggtgaaggagtgcgcccagctgggcaggctgccggtggtgaacttcgccgccggcggcgtggccaccccggccgacgccgccctgatgatgcagctgggctgcgacggcgtgttcgtgggcagcggcatcttcctgagcggcgacccggccaagagggccagggccatcgtgagggccgtgacccactacaacgacccgaagatcctggccgaggtgagcgagaacctgggcgccgccatggtgggcaggagcgtgagcagcctggaggagaaggagaagctggccaccaggggctggtga

SEQ ID NO: 120 - protein, 296 aa, Schizosaccharomyces pombe
MSAEIKGSTQVKAGLAQMLKGGVIMDVVNAEQARIAEAAGACAVMALERVPADIRAQGGVARMSDPSMIKEIQAAVSIPVMAKVRIGHFVEAQILESIGVDYIDESEVLTPADDINHIEKSKFTVPFVCGSRNLGEALRRISEGAAMIRTKGEAGTGDVVEAVRHTRQMQGELRRVKSMSPDELYTYAKEIAAPIDLVKECAQLGRLPVVNFAAGGVATPADAALMMQLGCDGVFVGSGIFLSGDPAKRARAIVRAVTHYNDPKILAEVSENLGAAMVGRSVSSLEEKEKLATRGW

SEQ ID NO: 121 - DNA, 986 bp, Petroselinum crispum
  promoter (1)..(986): Ubiquitin promoter
ctagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

SEQ ID NO: 122 - DNA, 1112 bp, Artificial
  synthetic sequence
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 123 - DNA, 695 bp, Vicia faba
  promoter (1)..(695): USP promoter variant used for expression of yeast genes in plants
ctagactgcagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcataccattaattaag

SEQ ID NO: 124 - DNA, 674 bp, Vicia faba
  promoter (1)..(674): USP promoter used for expression of bacterial genes in plants
caaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagc

SEQ ID NO: 125 - DNA, 102 bp, Arabidopsis thaliana
  CDS (1)..(102)
  transit_peptide (1)..(81): for targeting translational fusions to the mitochondria
atgcagaggtttttctccgccagatcgattctcggttacgccgtcaagacgcggaggaggtctttctcttctcgttcttcgtctctcctttgctcttccatg

SEQ ID NO: 126 - protein, 34 aa, Arabidopsis thaliana
MQRFFSARSILGYAVKTRRRSFSSRSSSLLCSSM

SEQ ID NO: 127 - DNA, 102 bp, Arabidopsis thaliana
  CDS (1)..(102)
  transit_peptide (1)..(81): for targeting translational fusions to the mitochondria
atgcagaggtttttctccgccagatcgattctcggttacgccgtcaagacgcggaggaggtctttctcttctcgttcttcggaattccagctgaccaccatg

SEQ ID NO: 128 - protein, 34 aa, Arabidopsis thaliana
MQRFFSARSILGYAVKTRRRSFSSRSSEFQLTTM

SEQ ID NO: 129 - DNA, 419 bp, Spinacia oleracea
  5′UTR (1)..(165): 5′ untranslated region
gcataaacttatcttcatagttgccactccaatttgctccttgaatctcctccacccaatacataatccactcctccatcacccacttcactactaaatcaaacttaactctgtttttctctctcctcctttcatttcttattcttccaatcatcgtactccgccatgaccaccgctgtcaccgccgctgtttctttcccctctaccaaaaccacctctctctccgcccgaagctcctccgtcatttcccctgacaaaatcagctacaaaaaggtgattcccaatttcactgtgttttttattaataatttgttattttgatgatgagatgattaatttgggtgctgcaggttcctttgtactacaggaatgtatctgcaactgggaaaatgggacccatcagggcccagatcgcctct

SEQ ID NO: 130 - protein, 59 aa, Spinacia oleracea
MTTAVTAAVSFPSTKTTSLSARSSSVISPDKISYKKVPLYYRNVSATGKMGPIRAQIAS

(19) United States
(12) Patent Application Publication
(10) Pub. No.: US 2011/0145948 A1
(43) Pub. Date: Jun. 16, 2011

(54) Transgenic Plants Comprising as Transgene a Phosphatidate Cytidylyltransferase

(75) Inventors: Bryan McKersie, Raleigh, NC (US); Wesley Bruce, Raleigh, NC (US)

(73) Assignee: BASG Plant Science GmbH, Ludwigshafen (DE)

(21) Appl. No.: 13/058,865

(22) PCT Filed: Aug. 10, 2009
(86) PCT No.: PCT/EP2009/060332

§ 371 Date: Feb. 14, 2011

Related U.S. Application Data
(60) Provisional application No. 61/090,308, filed on Aug. 20, 2008.
(60) Provisional application No. 61/090,625, filed on Aug. 21, 2008.
(60) Provisional application No. 61/090,669, filed on Aug. 21, 2008.

(51) Int. Cl. C12N 15/82; A01H 5/00; C07H 21/00

(52) U.S. Cl. 800/287; 800/278; 800/298; 536/23.2

(57) ABSTRACT

Polynucleotides are disclosed which are capable of enhancing yield of a plant transformed to contain such polynucleotides. Also provided are methods of using such polynucleotides and transgenic plants and agricultural products, including seeds, containing such polynucleotides as transgenes.

This application claims the priority benefit of U.S. provisional patent application Ser. No. 61/090,308, filed Aug. 20, 2008; U.S. provisional patent application Ser. No. 61/090,625, filed Aug. 21, 2008; and U.S. provisional patent application Ser. No. 61/090,669, filed Aug. 21, 2008. The entire contents of each of the above-referenced applications is incorporated herein by reference.

### FIELD OF THE INVENTION

This invention relates generally to transgenic plants which overexpress isolated polynucleotides that encode polypeptides active in lipid metabolism, in specific plant tissues and organelles, thereby improving yield of said plants.

### BACKGROUND OF THE INVENTION

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass. For example, water use efficiency (WUE), is a parameter often correlated with drought tolerance. Studies of a plant's response to desiccation, osmotic shock, and temperature extremes are also employed to determine the plant's tolerance or resistance to abiotic stresses.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field

Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Plant membranes contain diverse molecular species and the composition of these membranes change in response to environmental cues during acclimation processes and as a consequence of cellular injury from environmental stress. Plant membranes are generally considered to be a primary site of injury following exposure to low temperature stress and various forms of oxidative stress such as occur during water deprivation. This degradation may involve the action of specific hydrolytic enzymes or may be the consequence of oxidative reactions mediated by free radicals. In the case of oxidative free radical reactions, degradation may occur at the unsaturated double bonds of the fatty acid acyl chain or at the ester bond linking the fatty acid acyl chain to the glycerol backbone of the phospholipid. This degradation if sufficiently severe will promote cell death, but in more moderate circumstances, degradation products are components of cell signaling mechanisms that promote an acclimation response. During the acclimation process, plants adapt and develop greater tolerance of environmental stress. Coincidentally, plants generally alter both the quantity of membrane components in each cell and the composition of those membranes. These alterations in composition are coincident with increased tolerance of the whole plant. Common changes include changes in fatty acid unsaturation and phospholipid head groups. In addition, genetic differences among plants contribute to both differences in membrane composition, stress signaling mechanisms and the whole plant's ability to tolerate stress. Thus plant membranes are considered to be a central site for perceiving, tolerating and responding to environmental stress.

Phospholipids are the major structural components of biological membranes and also serve as important-signaling molecules. Phospholipids are commonly synthesized in the endoplasmic reticulum and transported to other membranes, but phospholipids can be synthesized in other cell compartments, including the mitochondria and chloroplast. Phospholipases hydrolyze phospholipids, and in plants, three classes of phospholipases have been reported including phospholipase A (PLA), phospholipase C (PLC), and phospholipase D (PLD). PLCs hydrolyze phosphotidylinositol 4,5-bisphosphate (PIP2) to inositol 1,4,5-triphosphate and diacylglycerol, which are components of the inositol signaling pathway.

As set forth above, plants can acclimate to environmental stress through moderate increases in the activity of enzymes that alter fatty acid oxidation. Most eukaryotic cells have two fatty-acid beta-oxidation systems, one in mitochondria and the other in peroxisomes. The Escherichia coli gene B2341 encodes a bifunctional anaerobic fatty acid oxidation complex protein associated with both enoyl-CoA hydratase and 3-hydroxyacyl-CoA epimerase activity. WO 2006/069610 and WO 2007/087815 disclose metabolic changes in plants transformed with the E. coli gene B2341 (SEQ ID NO:21).

Many enzymes exist as proenzymes or zymogens that require activation by a non-protein molecule, or cofactor, in order to exhibit full activity. Cofactors may be loosely categorized as coenzymes, prosthetic groups, or metal activators. A coenzyme is a small, heat-stable organic molecule that readily dissociates from a proenzyme that functions as a carrier of chemical groups between enzymes. Prosthetic groups are firmly bound to the proenzyme and form a permanent part of the protein structure.

The vitamin riboflavin is a component of the coenzymes flavin adenine dinucleotide (FAD) and flavin mononucleotide (FMN), which are required in the enzymatic oxidation of carbohydrates and other electron transport reactions critical for plants in their response to environmental stress conditions. Biosynthesis of riboflavin requires GTP and ribulose-5-phosphate (R5P) as precursors. The microbial riboflavin biosynthesis genes RibA-RibF have been cloned and biochemically characterized.

Homologs of RibA, RibB and RibE have been cloned from plants, and based on sequence analysis, the subcellular localization of the plant proteins has been deduced. Proteins that function in the plastid have a typical amino acid domain at the N terminus of the protein that acts as a targeting sequence to direct the protein into the plastid. The plant enzymes involved in riboflavin synthesis contain this plastid targeting sequence, whereas those that encode proteins involved in the biosynthesis of FAD from riboflavin do not. Therefore, the synthesis of the coenzymes FMN and FAD in plants is believed to occur sequentially in the plastid and the cytosol compartments. In wild type plants, the conversion of GTP to 5-amino-6-ribitylamino-2,4(1H,3H)-pyrimidinedione (ARP) occurs by several enzymatic reactions that are localized in plastids. The plastid compartment contains a pentose phosphate pathway which forms R5P. Within the plastid, R5P is metabolized to 3,4-dihydroxy-2-butanone 4-phosphate (DBP) which is combined by the enzymatic action of 6,7-dimethyl-8-ribityllumazine synthase (RibH) to form 6,7-dimethyl-8-ribityllumazine (DR). DR is converted to riboflavin by the enzyme riboflavin synthase in the plastid and then transported to the cytosol via an unknown mechanism. In the cytosol, riboflavin is phosphorylated to FMN and converted to FAD. FIG. 9 depicts the compartmentalized riboflavin and FAD biosynthetic pathway in wild type plants.

Enzyme I of FIG. 9 is GTP cyclohydrolase II (EC 3.4.5.25), or RibA, which catalyzes the first step in riboflavin synthesis. RibA is sometimes found as a bifunctional enzyme with 3,4-dihydroxy-2-butanone 4-phosphate synthase (DHBP_synthase) activity in addition to GTP cyclohydrolase II activity.

Enzyme VIII of FIG. 9 is RibH (also known as RibE, riboflavin synthase subunit beta, and lumazine synthase). U.S. Pat. Nos. 6,146,866 and 6,323,013 disclose the cloning of lumazine synthase from spinach, tobacco, Arabidopsis, and Magnaporthe grisea. The chloroplast targeting sequences of the spinach, tobacco, and Arabidopsis RibH polypeptides are identified in U.S. Pat. Nos. 6,146,866 and 6,323,013.

A second class of cofactors is the vitamin group known as vitamin B6. Three compounds belong to the vitamin group: pyridoxal, pyridoxine, and pyridoxamine, all of which are widely distributed in animals and plants, especially in cereal grains. Pyridoxal and pyridoxamine also occur in nature as their phosphate derivatives, pyridoxal 5′-phosphate (PLP) and pyridoxamine 5′-phosphate (PMP), which are the coenzyme forms of the vitamin. PLP participates in catalysis of several important reactions of amino acid metabolism, such as transamination, decarboxylation, and racemization.

De novo synthesis of PLP occurs only in bacteria, fungi, and plants. In Escherichia coli, de novo synthesis occurs through condensation of 4-phosphohydroxy-L-threonine and deoxyxyulose 5-phosphate to form pyridoxine 5′-phosphate (PNP). The condensation reaction is catalyzed by the concerted action of the PdxA and PdxJ enzymes. In the E. coli de novo pathway, PNP is then oxidized by the PdxH oxidase to form PLP. Recently a different de novo PLP biosynthetic pathway has been identified which is independent of deoxyxyulose 5-phosphate. In this pathway, PLP is synthesized from ribose 5-phosphate or R5P and either glyceraldehyde 3-phosphate or dihydroxyacetone phosphate, via the products of two genes designated PDX1 and PDX2, which show no homology to any of the E. coli PLP synthetic genes. The PDX1 and PDX2 gene products are predicted to function as a glutamine amidotransferase, with PDX2 as the glutaminase domain and PDX1 as the acceptor/synthase domain. The pdx1 gene product of the filamentous fungus Cercospora nicotianae is highly homologous to a conserved gene family designated SOR1, which is widespread in archeabacteria, eubacteria, plants, and fungi. The two pathways of PLP de novo synthesis are autoexclusive, that is, organisms have the genes for one or the other pathway, but not both.

PLP may also be synthesized by a second pathway, designated a salvage pathway, through which pyridoxal (PL), pyridoxine (PN), and pyridoxamine (PM) taken up from the cell's growth medium. The salvage pathway is present in addition to the de novo synthetic pathway in E. coli, and is the only means by which mammalian cells can make PLP. In the E. coli salvage pathway, PL, PN, and PM are first phosphorylated by kinases to form PLP, pyridoxine 5′-phosphate (PNP), and PMP, respectively. PNP and PMP are oxidized by the PdxH oxidase referenced above. The pdxK gene encodes a PN/PL/PM kinase, and the pdxY gene encodes a PL kinase, and the products of both genes share a number of conserved motifs with the PfkB superfamily of carbohydrate kinases. Homologs of the PdxK and PdxY kinases have been identified from humans, Trypanosoma brucei, Haemophilus influenzae, Caenorhabditis elegans, Rattus norvegicus, Saccharomyces cerevisiae, and Salmonella typhimurium.

Although some genes that are involved in stress responses, water use, and/or biomass in plants have been characterized, to date, success at developing transgenic crop plants with improved yield has been limited, and no such plants have been commercialized. There is a need, therefore, to identify additional genes that have the capacity to increase yield of crop plants.

### SUMMARY OF THE INVENTION

The present inventors have discovered that there are three critical components that must be optimized to achieve improvement in plant yield through transgenic expression of certain polypeptides When targeted under the regulatory elements as described herein, the polynucleotides and polypeptides set forth in Table 1 are capable of improving yield of transgenic plants

TABLE 1

| | | | Amino |
|---|---|---|---|
| | | | acid |
| | | Polynucleotide | SEQ |
| Gene Name | Organism | SEQ ID NO: | ID NO: |
| YBR029C | S. cerevisiae | 1 | 2 |
| BN04MC30805 | Brassica napus | 3 | 4 |
| ZM06MC30283 | Zea mays | 5 | 6 |
| YKL192C | S. cerevisiae | 7 | 8 |
| BN1004MS43616414 | B. napus | 9 | 10 |
| GM06MC07589 | G. max | 11 | 12 |
| HA1004MS66693619 | Helianthus annuus | 13 | 14 |
| YDR018C | S. cerevisiae | 15 | 16 |
| GM06MC27072 | G. max | 17 | 18 |
| ZM06MC04863 | Z. mays | 19 | 20 |
| B2341 | E. coli | 21 | 22 |
| ZM06MC04303 | Z. mays | 23 | 24 |
| ZM06MC15742 | Z. mays | 25 | 26 |
| B0452 | E. coli | 27 | 28 |
| BN42634969 | B. napus | 29 | 30 |
| BNP5302_30 | B. napus | 31 | 32 |
| GMsae90f11 | G. max | 33 | 34 |
| YNL202W | S. cerevisiae | 35 | 36 |
| HA66688442 | Helianthus annuus | 37 | 38 |
| YKL140W | S. cerevisiae | 39 | 40 |
| SLL1023 | Synechocystis sp. | 41 | 42 |
| | PCC 6803 | | |
| SLR0252 | Synechocystis sp. | 43 | 44 |
| b3803 | E. coli | 45 | 46 |
| BN51286476 | B. napus | 47 | 48 |
| GM59791864 | G. max | 49 | 50 |
| ZMBFb0243J04 | Z. mays | 51 | 52 |
| b3209 | E. coli | 53 | 54 |
| GMss34d01 | G. max | 55 | 56 |
| HA03MC1392 | H. annuus | 57 | 58 |
| b2578 | E. coli | 59 | 60 |
| b2682 | E. coli | 61 | 62 |
| b3285 | E. coli | 63 | 64 |
| b1938 | E. coli | 65 | 66 |
| SLL1894 | S. sp. PCC 6803 | 67 | 68 |
| GM08000037 | G. max | 69 | 70 |
| SLL1282 | S. sp. PCC 6803 | 71 | 72 |
| GM06MC29296 | G. max | 73 | 74 |
| ZM07MC00430 | Z. mays | 75 | 76 |
| ZM07MC23187 | Z. mays | 77 | 78 |
| b1636 | E. coli | 79 | 80 |
| pdxH | E. coli | 81 | 82 |
| ECpdxK | E. coli | 83 | 84 |
| TBpdxK | Trypanosoma brucei | 85 | 86 |
| CEpdxK | Caenorhabditis | 87 | 88 |
| | elegans | | |
| STyfei | Salmonella | 89 | 90 |
| | typhimurium | | |
| HIyfei | Haemophilus. | 91 | 92 |
| | influenzae | | |
| Yn8fp | S. cerevisiae | 93 | 94 |
| SLR1779 | S. sp. PCC 6803 | 95 | 96 |
| pdxJ | E. coli | 97 | 98 |
| pdx1.1 | A. thaliana | 99 | 100 |
| pdx1.3 | A. thaliana | 101 | 102 |
| CNpdx1 | Cercospora | 103 | 104 |
| | nicotianae | | |
| SCpdx1 | S. cerevisiae | 105 | 106 |
| BSpdx1 | Bacillus subtilis | 107 | 108 |
| OSpdx1 | Oryza sativa | 109 | 110 |
| HBpdx1 | Hevea brasiliensis | 111 | 112 |
| SLpdx1 | Stellaria longipes | 113 | 114 |
| BNpdx1 | B. napus | 115 | 116 |
| PPpdx1 | Physcomitrella patens | 117 | 118 |
| SPpdx1 | Schizosaccharomyces | 119 | 120 |
| | pombe | | |

In one embodiment, the invention provides a transgenic plant transformed with an expression cassette comprising, in operative association, an isolated polynucleotide encoding a promoter capable of enhancing gene expression in roots and shoots; an isolated polynucleotide encoding a subcellular targeting peptide; and an isolated polynucleotide encoding a full-length phosphatidate cytidylyltransferase polypeptide, wherein the transgenic plant demonstrates increased yield as compared to a wild type plant of the same variety which does not comprise the expression cassette.

In another embodiment, the invention provides a transgenic plant transformed with an expression cassette comprising in operative association, an isolated polynucleotide encoding a promoter capable of enhancing expression in leaves; an isolated polynucleotide encoding a mitochondrial transit peptide; and an isolated polynucleotide encoding a full-length acyl-carrier protein, wherein the transgenic plant demonstrates increased yield as compared to a wild type plant of the same variety which does not comprise the expression cassette.

In another embodiment, the invention provides a transgenic plant transformed with an expression cassette comprising, in operative association, isolated polynucleotide encoding a promoter; an isolated polynucleotide encoding a subcellular targeting peptide; and an isolated polynucleotide encoding a full-length acyltransferase polypeptide; wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length bifunctional anaerobic fatty acid oxidation complex polypeptide, regenerating transgenic plants from the transformed plant cell, and selecting higher-yielding plants from the transgenic plants.

In another embodiment, the invention provides a method of increasing yield of a plant by transforming a wild-type plant with an expression cassette comprising, in operative association, an isolated polynucleotide encoding a promoter capable of enhancing gene expression in leaves; an isolated polynucleotide encoding a subcellular targeting peptide; and an isolated polynucleotide encoding a full-length acyl-CoA thioesterase polypeptide; wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter; an isolated polynucleotide encoding a subcellular targeting peptide; and an isolated polynucleotide encoding a full-length sterol esterase polypeptide; wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter capable of enhancing gene expression in leaves; an isolated polynucleotide encoding a mitochondrial targeting peptide; and an isolated polynucleotide encoding a full-length 2,4-dienoyl-CoA reductase polypeptide; wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter; an isolated polynucleotide encoding a plastid transit peptide; and an isolated polynucleotide encoding a full-length succinate-CoA ligase polypeptide; wherein the transgenic plant demonstrates increased yield as compared to a wild type plant of the same variety which does not comprise the expression cassette.

In another embodiment, the invention provides a method of increasing yield of a plant by transforming a wild-type plant with an expression cassette comprising, in operative association, an isolated polynucleotide encoding a promoter and an isolated polynucleotide encoding a full-length cobalt-precorrin-6A reductase polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length polypeptide having uroporphyrin-III C-methyltransferase activity and a HemX signature sequence; wherein the transgenic plant demonstrates increased yield as compared to a wild type plant of the same variety which does not comprise the expression cassette.

In another embodiment, the invention provides a transgenic plant transformed with an expression cassette comprising in operative association, an isolated polynucleotide encoding a promoter capable of enhancing gene expression in roots and shoots and an isolated polynucleotide encoding a full-length polypeptide having isoprenoid biosynthesis activity and a DJ-1_Pfp1 signature sequence; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in roots and shoots; and an isolated polynucleotide encoding a full-length polypeptide having LysE type translocator activity and a LysE signature sequence comprising amino acids 14 to 195 of SEQ ID NO:60; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in roots and shoots and an isolated polynucleotide encoding a full-length polypeptide having LIV-E family branched-chain amino acid transport activity and an AzIC signature sequence; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in roots and shoots; and an isolated polynucleotide encoding a truncated DNA-binding polypeptide having a sequence comprising amino acids 1 to 102 of SEQ ID NO:64; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in roots and shoots and an isolated polynucleotide encoding a full-length polypeptide having a first YscJ_FliF signature sequence and a second YscJ_FliF signature sequence; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; and an isolated polynucleotide encoding a full length GTP cyclohydrolase II polypeptide which does not comprise a subcellular targeting peptide; wherein the transgenic plant demonstrates increased yield as compared to a wild type plant of the same variety that does not comprise the expression cassette.

In another embodiment, the invention provides a transgenic plant transformed with an expression cassette comprising, in operative association, an isolated polynucleotide encoding a promoter and an isolated polynucleotide encoding a full length lumazine synthase polypeptide which does not comprise a subcellular targeting peptide; wherein the transgenic plant demonstrates increased yield as compared to a wild type plant of the same variety that does not comprise the expression cassette.

In another embodiment, the invention provides a transgenic plant transformed with an expression cassette comprising, in operative association, an isolated polynucleotide encoding a promoter and an isolated polynucleotide encoding a full length polypeptide capable of enhancing pyridoxal 5′-phosphate synthesis; wherein the transgenic plant demonstrates increased yield as compared to a wild type plant of the same variety that does not comprise the expression cassette. In this embodiment, the expression cassette may further comprise a polynucleotide encoding a mitochondrial or plastid transit peptide.

In a further embodiment, the invention provides a seed produced by the transgenic plants described above, wherein the seed is true breeding for a transgene comprising the expression vectors described above. Plants derived from the seed of the invention demonstrate increased tolerance to an environmental stress, and/or increased plant growth, and/or increased yield, under normal or stress conditions as compared to a wild type variety of the plant.

In a still another aspect, the invention concerns products produced by or from the transgenic plants of the invention, their plant parts, or their seeds, such as a foodstuff, feedstuff, food supplement, feed supplement, fiber, cosmetic or pharmaceutical.

The invention further provides certain isolated polynucleotides identified in Table 1, and certain isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of Table 1, and generating a transgenic plant from the plant cell, wherein the transgenic plant comprises the polynucleotide.

### BRIEF DESCRIPTION OF THE DRAWINGS

FIG. 1 shows an alignment of the amino acid sequences of full-length phosphatidate cytidylyltransferase polypeptide designated YBR029C (SEQ ID NO:2), BN04MC30805 (SEQ ID NO:4) and ZM06MC30283 (SEQ ID NO:6) The alignment was generated using Align X of Vector NTI Advance 10.3.0.

FIG. 2 shows an alignment of the amino acid sequences of the acyl-carrier proteins designated YKL192C (SEQ ID NO:8), BN1004MS43616414 (SEQ ID NO:10), GM06MC07589(SEQ ID NO:12), and HA1004MS66693619 (SEQ ID NO:14). The alignment was generated using Align X of Vector NTI Advance 10.3.0.

FIG. 3 shows an alignment of the amino acid sequences of the acyltransferases designated: YDR018C (SEQ ID NO:16), GM06MC27072 (SEQ ID NO:18) and ZM06MC04863 (SEQ ID NO:20). The alignment was generated using Align X of Vector NTI Advance 10.3.0.

FIG. 4 shows an alignment of the amino acid sequences encoding bifunctional anaerobic fatty acid oxidation complex polypeptides designated b2341 (SEQ ID NO:22), ZM06MC04303 (SEQ ID NO:24) and ZM06MC15742 (SEQ ID NO:26). The alignment was generated using Align X of Vector NTI Advance 10.3.0.

FIG. 5 shows an alignment of the amino acid sequences encoding acyl-CoA thioesterase polypeptides designated B0452 (SEQ ID NO:28), BN42634969 (SEQ ID NO:30), BNP5302_{—}30 (SEQ ID NO:32), and GMsae90f11 (SEQ ID NO:34). The alignment was generated using Align X of Vector NTI Advance 10.3.0.

FIG. 6 shows an alignment of the amino acid sequences encoding 2,4-dienoyl-CoA reductase polypeptides designated YNL202W (SEQ ID NO:36) and HA66688442 (SEQ ID NO:38). The alignment was generated using Align X of Vector NTI Advance 10.3.0.

FIG. 7 shows an alignment of the amino acid sequences of uroporphyrin-III C-methyltransferases designated b3803 (SEQ ID NO:46), BN51286476 (SEQ ID NO:48), GM59791864 (SEQ ID NO:50), and ZMBFb0243J04 (SEQ ID NO:52). The alignment was generated using Align X of Vector NTI Advance 10.3.0.

FIG. 8 shows an alignment of the amino acid sequences of the isoprenoid biosynthesis proteins designated b3209 (SEQ ID NO:54), GMss34d01 (SEQ ID NO:56), and HA03MC1392 (SEQ ID NO:58). The alignment was generated using Align X of Vector NTI Advance 10.3.0.

FIG. 9 shows a flow diagram of the riboflavin/FAD biosynthesis pathway in wild type plants. Enzyme designations are as follows: Enzyme I is GTP cyclohydrolase II (RibA); Enzyme II is 2,5-diamino-6-ribosylamino-4(3H)-pyrimidinone 50-phosphate deaminase; Enzyme III is 5-amino-6-ribosylamino-2,4(1H,3H)-pyrimidinedione 50-phosphate reductase; Enzyme IV is 2,5-diamino-6-ribosylamino-4(3H)-pyrimidinone 50-phosphate reductase; Enzyme V is 2,5-diamino-6-ribitylamino-4(3H)-pyrimidinedione 50-phosphate deaminase; Enzyme VI is a hypothetical phosphatase; Enzyme VII is 3,4-dihydroxy-2-butanone-4-phosphate synthase; Enzyme VIII is 6,7-dimethyl-8-ribityllumazine synthase (RibH); Enzyme IX is riboflavin synthase; Enzyme X is riboflavin kinase; and Enzyme XI is FAD synthetase. Intermediates in the biosynthesis of riboflavin and FAD are GTP; DAPP (2,5-diamino-6-ribosylamino-4(3H)-pyrimidinone 50-phosphate); AAPP (5-amino-6-ribosylamino-2,4(1H,3H)-pyrimidinedione 50-phosphate); DRPP (2,5-diamino-6-ribitylamino-4(3H)-pyrimidinedione 50-phosphate); ARPP (5-amino-6-ribitylamino-2,4(1H,3H)-pyrimidinedione 50-phosphate); ARP; G6P (Glucose-6-phosphate); riboflavin; with RSP, DBP, DR, FMN, and FAD as set forth above.

FIG. 10A shows a flow diagram of the proposed riboflavin/FAD biosynthesis pathway in the transgenic plants of the invention. Abbreviations are as set forth in FIG. 9. FIG. 10A shows the pathway with RibH targeted to the cytosol. FIG. 10B shows the pathway with RibA targeted to the cytosol; FIG. 10C shows the pathway with both RibH and RibA targeted to the cytosol.

FIG. 11 shows a flow diagram of vitamin B6 synthesis as it relates to the present invention. Abbreviations: DAP: dihydroxyacetone-P; G3P: glyceraldehyde-3P; with PL, PLP, PM, PN, PNP, and R5P as set forth above.

FIG. 12 shows an alignment of the amino acid sequences of GTP cyclohydrolase II designated SLL1894 (SEQ ID NO:68) and Gm018000037 (SEQ ID NO:70), The alignment was generated using Align X of Vector NTI Advance 10.3.0. Amino acids 1 to 49 of SEQ ID NO:70 correspond to the subcellular targeting sequence.

FIG. 13 shows an alignment of the amino acid sequences of the lumazine synthase polypeptides designated SLL1282 (SEQ ID NO:72), GM06MC29296 (SEQ ID NO:74), ZM07MC00430 (SEQ ID NO:76) and ZM07MC23187 (SEQ ID NO:78). The alignment was generated using Align X of Vector NTI. Advance 10.3.0. Subcellular targeting sequences correspond to amino acids 1 to 53 of SEQ ID NO:74; amino acids 1 to 56 of SEQ ID NO:76; and amino acids 1 to 80 of SEQ ID NO:78.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, reduction of nutrient, e.g., nitrogen or phosphorus, input requirement, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, is an improved yield in accordance with the invention.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli); and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are A. thaliana, Nicotiana tabacum, rice, oilseed rape, canola, soybean, corn (maize), cotton, and wheat.

### A. Phosphatidate Cytidylyltransferase

In one embodiment, the invention provides a provides a transgenic plant transformed with an expression cassette comprising, in operative association, an isolated polynucleotide encoding a promoter capable of enhancing gene expression in roots and shoots; an isolated polynucleotide encoding a subcellular targeting peptide; and an isolated polynucleotide encoding a full-length phosphatidate cytidylyltransferase polypeptide, wherein the transgenic plant demonstrates increased yield as compared to a wild type plant of the same variety which does not comprise the expression cassette. In accordance with the invention, when the promoter is capable of enhancing expression in roots or shoots, the subcellular targeting peptide is a plastid transit peptide.

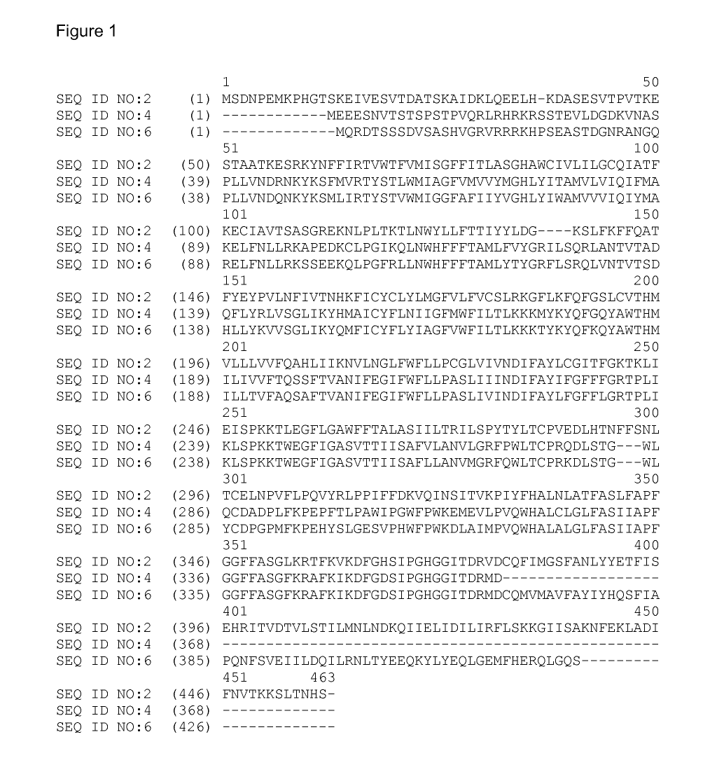

As indicated in Table 2 below, when the S. cerevisiae gene product YBR029C (SEQ ID NO:2) is targeted to the chloroplast and the gene's transcriptional expression is driven by the Super promoter, transgenic plants demonstrate improved response to water-limiting conditions. Moreover, Table 3 indicates that under well-watered conditions, plants expressing YBR029C which is targeted to the plastid or to mitochondria were larger than control plants. Gene YBR029C encodes a phosphatidate cytidylyltransferase (EC 2.7.7.41), also known as CDP-diacylglycerol synthase (CDS), which catalyzes the synthesis of CDP-diacylglycerol from CTP and phosphatidate. CDS is a membrane-bound protein, with eight predicted membrane spanning regions in potato and Arabidopsis. Phosphatidate cytidylyltransferases are characterized, in part, by a distinctive signature sequence of “S-x-[LIVMF]-K-R-x(4)-K-D-x-[GSA]-x(2)-[LIF]-[PGS]-x-H-G-G-[LIVMF]-x-D-R-[LIVMFT]-D” where amino acid positions within square brackets can be any of the designated residues, and unbracketed amino acid positions can only be that specific amino acid residue. Such conserved signature sequences are exemplified in the phosphatidate cytidylyltransferase proteins set forth in FIG. 1.

The transgenic plant of this embodiment may comprise any polynucleotide encoding phosphatidate cytidylyltransferase. Preferably, the transgenic plant of this embodiment comprises a polynucleotide encoding a polypeptide having phosphatidate cytidylyltransferase activity, wherein the polypeptide comprises a phosphatidate cytidylyltransferase signature sequence selected from the group consisting of amino acids 351 to 377 of SEQ ID NO:2, amino acids 341 to 367 of SEQ ID NO:4, or amino acids 340 to 366 of SEQ ID NO:6. More preferably, the transgenic plant of this embodiment comprises a polynucleotide encoding phosphatidate cytidylyltransferase domain having a sequence comprising amino acids 65 to 377 of SEQ ID NO:2, amino acids 54 to 367 of SEQ ID NO:4, or amino acids 53 to 366 of SEQ ID NO:6. Most preferably, the transgenic plant of this embodiment comprises a polynucleotide encoding phosphatidate cytidylyltransferase polypeptide comprising amino acids 1 to 457 of SEQ ID NO:2, amino acids 1 to 367 of SEQ ID NO:4, or amino acids 1 to 425 of SEQ ID NO:6.

### B. Acyl Carrier Protein

In another embodiment, the invention provides a transgenic plant transformed with an expression cassette comprising in operative association, an isolated polynucleotide encoding a promoter capable of enhancing expression in leaves; an isolated polynucleotide encoding a mitochondrial transit peptide; and an isolated polynucleotide encoding an acyl carrier protein; wherein the transgenic plant demonstrates increased yield as compared to a wild type plant of the same variety which does not comprise the expression cassette.

As shown in Table 4 below, when the S. cerevisiae gene product YKL192C (SEQ ID NO:8) is targeted to the mitochondria under the control of the USP promoter (SEQ ID NO:123), transgenic plants demonstrate improved response to water-limiting conditions. Gene YKL192C encodes an acyl-carrier protein (ACP), containing a phosphopantetheine binding domain (PF00550). Acyl carrier proteins catalyze a condensation reaction to form peptide bonds in non-ribosomal protein biosynthesis. Acyl carrier protein is a universal and highly conserved carrier of acyl groups in fatty acid biosynthesis. The amino-terminal region of the ACP proteins is well defined and consists of alpha four helices arranged in a right-handed bundle held together by inter-helical hydrophobic interactions.

Phosphopantetheine (or pantetheine 4′ phosphate) is the prosthetic group of ACP in some multienzyme complexes, where it serves as a ‘swinging arm’ for the attachment of activated fatty acid and amino acid groups. Phosphopantetheine binding domains are characterized, in part, by the presence of the distinctive phosphopantetheine attachment site signature sequence, “[DEQGSTALMKRH]-[LIVMFYSTAC]-[GNQ]-[LIVMFYAG]-[DNEKHS]-S-[LIVMST]-{PCFY}-[STAGCPQLIVMF]-[LIVMATN]-[DENQGTAKRHLM]-[LIVMWSTA]-[LIVGSTACR]-{LPIY}-{VY}-[LIVMFA]” where amino acid positions within square brackets can be any of the designated residues, amino acid positions within curly brackets can be any amino acid residue except the one(s) listed and unbracketed amino acid positions can only be that specific amino acid residue. The phosphopantetheine moiety is attached to serine residue indicated in bold italic in the above signature sequence. This serine residue is present in the amino terminus of helix II, a domain of the protein referred to as the recognition helix and which is responsible for the interaction of ACPs with the enzymes of type II fatty acid synthesis.

Preferably, the transgenic plant of this embodiment comprises a polynucleotide encoding a full-length ACP comprising a phosphopantetheine binding site signature sequence selected from the group consisting of amino acids 77 to 92 of SEQ ID NO:8, amino acids 73 to 88 of SEQ ID NO:10, amino acids 75 to 90 of SEQ ID NO:12, or amino acids 84 to 99 of SEQ ID NO:14. More preferably, the transgenic plant of this embodiment comprises a polynucleotide encoding an acyl-carrier protein comprising an acyl carrier protein domain selected from the group consisting of amino acids 49 to 106 of SEQ ID NO:8, amino acids 49 to 102 of SEQ ID NO:10, amino acids 51 to 104 of SEQ ID NO:12, amino acids 60 to 113 of SEQ ID NO:14. Most preferably, the transgenic plant of this embodiment comprises a polynucleotide encoding an acyl-carrier protein comprising amino acids 1 to 125 of SEQ ID NO:8, amino acids 1 to 117 of SEQ ID NO:10, amino acids 1 to 128 of SEQ ID NO:12, or amino acids 1 to 119 of SEQ ID NO:14.

### C. Acyltransferase

In another embodiment, the invention provides a transgenic plant transformed with an expression cassette comprising, in operative association, isolated polynucleotide encoding a promoter; an isolated polynucleotide encoding a subcellular targeting peptide; and an isolated polynucleotide encoding an acyltransferase polypeptide; wherein the transgenic plant demonstrates increased yield as compared to a wild type plant of the same variety which does not comprise the expression cassette.

As set forth in Table 5 below, when the S. cerevisiae acyltransferase gene product YDR018C (SEQ ID NO:16) is targeted to the chloroplast or mitochondria under the control of the Super promoter or the USP promoter (SEQ ID NO:123), transgenic plants demonstrate improved response to water-limiting conditions. Moreover, Table 6 indicates that under well-watered conditions, plants expressing YDR018C under control of the USP promoter (SEQ ID NO:123), and targeted to the mitochondria, were larger than control plants.

The transgenic plant of this embodiment may comprise any polynucleotide encoding an acyltransferase polypeptide. Preferably, the transgenic plant of this embodiment comprises a polynucleotide encoding an acyltransferase polypeptide comprising an acyltransferase domain selected from the group consisting of amino acids 108 to 272 of SEQ ID NO:16, amino acids 80 to 222 of SEQ ID NO:18, or amino acids 116 to 258 of SEQ ID NO:20. More preferably, the transgenic plant of this embodiment comprises a polynucleotide encoding an acyltransferase having a sequence comprising amino acids 1 to 396 of SEQ ID NO:16, amino acids 1 to 384 of SEQ ID NO:18, or amino acids 1 to 332 of SEQ ID NO:20.

D. Bifunctional anaerobic fatty acid oxidation complex polypeptide

In another embodiment, the invention provides a method of increasing yield of a plant species by transforming a wild type cell of said species with an expression cassette comprising, in operative association, an isolated polynucleotide encoding a promoter capable of enhancing expression in leaves; an isolated polynucleotide encoding a mitochondrial transit peptide; and an isolated polynucleotide encoding a bifunctional anaerobic fatty acid oxidation complex polypeptide In a second step, transgenic plantlets are regenerated from the transformed plant cell. In a third step, the transgenic plantlets are subjected to a yield-related assay, and higher-yielding plants are selected from the regenerated transgenic plants.

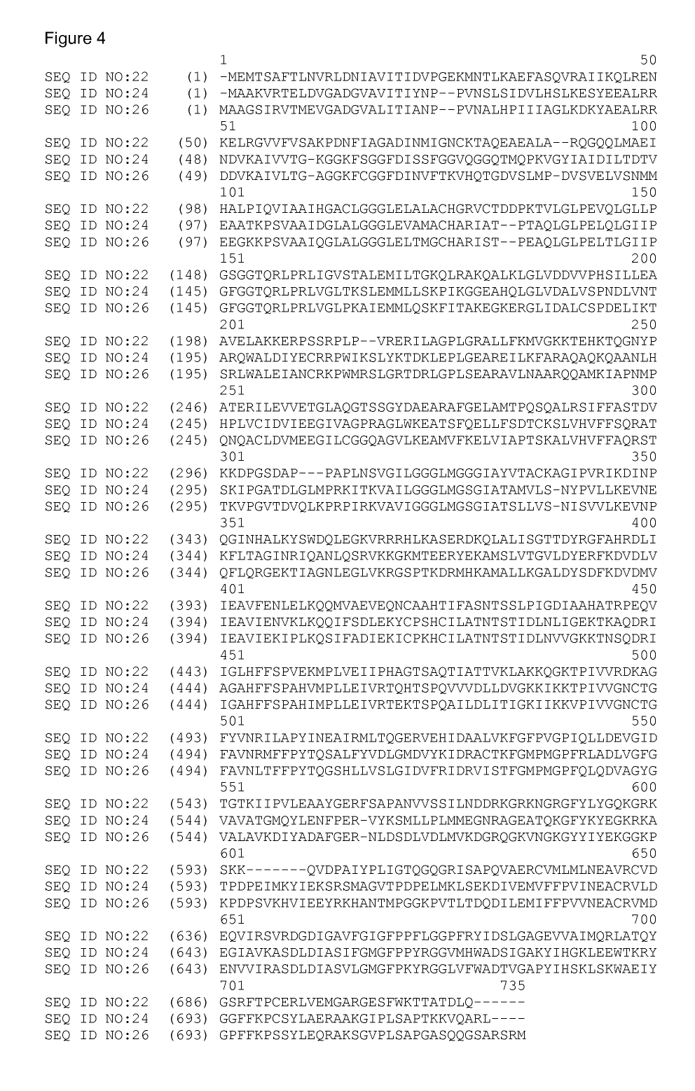

As shown in Tables 7 and 8 below, when transcription of the E. coli gene B2341 (SEQ ID NO:21) is under control of the USP promoter (SEQ ID NO:123) and the gene product (SEQ ID NO:22) is targeted to the mitochondria, transgenic plants tend to be larger and darker green than control plants. Gene B2341 encodes a protein that comprises three domains: a domain characteristic of the ECH or enoyl-CoA hydratase/isomerase family (PF00378); a C-terminal domain characteristic 3HCDH or 3-hydroxyacyl-CoA dehydrogenase (PF00725); and a 3HCDH_N,3-hydroxyacyl-CoA dehydrogenase, NAD binding domain (PF02737). The 3HCDH domain is characterized, in part, by the presence of the signature sequence, “[DNES]-x(2)-[GA]-F-[LIVMFYA]-x-[NT]-R-x(3)-[PA]-[LIVMFY]-[LIVMFYST]-x(5,6)-[LIVMFYCT]-[LIVMFYEAH]-x(2)-[GVE]”, where amino acid positions within square brackets can be any of the designated residues, and unbracketed amino acid positions can only be that specific amino acid residue. All the sequences shown in FIG. 4 exhibit this characteristic signature sequence, the only deviation within this group is the presence of a leucine residue at position 498 in ZM06MC15742 (SEQ ID NO:26) instead of the canonical arginine residue.

The method of this embodiment may employ any polynucleotide encoding a bifunctional anaerobic fatty acid oxidation complex polypeptide comprising an ECH domain, a 3HCDH-C terminal domain and a 3HCHD-NAD binding domain. Preferably, the method of this embodiment employs a polynucleotide encoding a bifunctional anaerobic fatty acid oxidation complex polypeptide comprising three domains: a) an ECH domain selected from the group consisting of amino acids 17 to 190 of SEQ ID NO:22; amino acids 17 to 187 SEQ ID NO:24 and amino acids 18 to 187 SEQ ID NO:26; b) a 3HCDH domain selected from the group consisting of amino acids 489 to 513 of SEQ ID NO:22, amino acids 490 to 514 of SEQ ID NO:24, amino acids 490 to 514 of SEQ ID NO:26; and c) a 3HCDH-N domain selected from the group consisting of amino acids 310 to 490 of SEQ ID NO:22; amino acids 312 to 491 of SEQ ID NO:24 and amino acids 312 to 491 SEQ ID NO:26 More preferably, the method of this embodiment employs a polynucleotide encoding a bifunctional anaerobic fatty acid oxidation complex polypeptide having a sequence comprising amino acids 1 to 714 of SEQ ID NO:22, amino acids 1 to 723 of SEQ ID NO:24, or amino acids 1 to 727 of SEQ ID NO:26.

The invention is also embodied in a transgenic plant comprising a polynucleotide encoding a bifunctional anaerobic fatty acid oxidation complex polypeptide comprising a) an ECH domain selected from the group consisting of amino acids 17 to 190 of SEQ ID NO:22; amino acids 17 to 187 SEQ ID NO:24 and amino acids 18 to 187 SEQ ID NO:26; b) a 3HCDH domain selected from the group consisting of amino acids 489 to 513 of SEQ ID NO:22, amino acids 490 to 514 of SEQ ID NO:24, amino acids 490 to 514 of SEQ ID NO:26; and c) a 3HCDH-N domain selected from the group consisting of amino acids 310 to 490 of SEQ ID NO:22; amino acids 312 to 491 of SEQ ID NO:24 and amino acids 312 to 491 SEQ ID NO:26 More preferably, the transgenic plant of this embodiment comprises a polynucleotide encoding a bifunctional anaerobic fatty acid oxidation complex polypeptide having a sequence comprising amino acids 1 to 714 of SEQ ID NO:22, amino acids 1 to 723 of SEQ ID NO:24, or amino acids 1 to 727 of SEQ ID NO:26.

### E. Acyl-CoA Thioesterase

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length acyl-CoA thioesterase polypeptide; wherein the transgenic plant demonstrates increased yield as compared to a wild type plant of the same variety which does not comprise the expression cassette.

As shown in Table 9 below, when transcription of the E. coli gene b0452 is targeted to mitochondria under the control of the USP promoter, transgenic plants were larger than control plants, both under well-watered and drought conditions. Gene B0452 (SEQ ID NO:27) encodes an acyl-CoA thioesterase (EC 3.1.2.2). Acyl-CoA thioesterases (ACH) are a group of enzymes that catalyze the hydrolysis of acyl-CoAs to the free fatty acid and coenzyme A (CoASH), providing the potential to regulate intracellular levels of acyl-CoAs, free fatty acids and CoASH. This enzyme displays high levels of activity on medium- and long chain acyl CoAs. Two families of ACHs have been identified in A. thaliana. One family, consisting of AtACH1 and AtACH2, appears to be peroxisomal, as they have type-1 peroxisomal targeting sequences. The other family, consisting of AtACH4 and AtACH5, resides in the endoplasmic reticulum.

The transgenic plant of this embodiment may comprise any polynucleotide encoding an acyl-CoA thioesterase. Preferably, the transgenic plant of this embodiment comprises a polynucleotide encoding a full-length polypeptide having acyl-CoA thioesterase activity, wherein the polypeptide comprises a domain selected from the group consisting of amino acids 107 to 184 of SEQ ID NO:28, amino acids 54 to 138 of SEQ ID NO:30, amino acids 127 to 211 of SEQ ID NO:32, and amino acids 3 to 87 of SEQ ID NO:34. More preferably, the transgenic plant of this embodiment comprises a polynucleotide encoding an acyl-CoA thioesterase having a sequence comprising amino acids 1 to 286 of SEQ ID NO:28, amino acids 1 to 248 of SEQ ID NO:30, amino acids 1 to 212 of SEQ ID NO:32, or amino acids 1 to 197 of SEQ ID NO:34.

F. 2,4-dienoyl-CoA Reductase

In another embodiment, the invention provides a transgenic plant transformed with an expression cassette comprising, in operative association, an isolated polynucleotide encoding a promoter capable of enhancing expression in leaves; an isolated polynucleotide encoding a mitochondrial transit peptide; and an isolated polynucleotide encoding a full-length 2,4-dienoyl-CoA reductase polypeptide; wherein the transgenic plant demonstrates increased yield as compared to a wild type plant of the same variety which does not comprise the expression cassette.

As shown in Table 10 below, when transcription of the S. cerevisiae gene YNL202W (SEQ ID NO:35) is targeted to mitochondria under control of the USP promoter, under cycling drought conditions, transgenic plants were larger than control plants. YNL202W is a 2,4-dienoyl-CoA reductase (EC 1.3.1.34, DECR), an auxiliary enzyme of beta-oxidation. DECR participates in the degradation of unsaturated fatty enoyl-CoA esters having double bonds in both even- and odd-numbered positions in peroxisome. It catalyzes the NADP-dependent reduction of 2,4-dienoyl-CoA to yield trans-3-enoyl-CoA.

The transgenic plant of this embodiment may comprise any polynucleotide encoding a 2,4-dienoyl-CoA reductase. Preferably, the transgenic plant of this embodiment comprises a polynucleotide encoding a full-length polypeptide having 2,4-dienoyl-CoA reductase activity, wherein the polypeptide comprises a domain selected from the group consisting of amino acids 107 to 270 of SEQ ID NO:36 and amino acids 54 to 227 of SEQ ID NO:38. More preferably, the transgenic plant of this embodiment comprises a polynucleotide encoding a 2,4-dienoyl-CoA reductase having a sequence comprising amino acids 1 to 296 of SEQ ID NO:36 or amino acids 1 to 264 of SEQ ID NO:38.

### G. Sterol Esterase

In another embodiment, the invention provides a transgenic plant transformed with an expression cassette comprising, in operative association, an isolated polynucleotide encoding a promoter; an isolated polynucleotide encoding a subcellular transit peptide; and an isolated polynucleotide encoding a full-length sterol esterase; wherein the transgenic plant demonstrates increased yield as compared to a wild type plant of the same variety which does not comprise the expression cassette.

As shown in Table 11 below, when transcription of the S. cerevisiae gene YKL140W (SEQ ID NO:39) is targeted to plastids under control of the PcUbi promoter, transgenic plants were larger than control plants under well-watered conditions. Table 11 also shows that when YKL140W transcription was targeted to mitochondria under control of the USP promoter, transgenic plants were larger than control plants under cycling drought conditions. YKL140W encodes a sterol esterase (EC 3.1.1.13). In yeast, sterol esterase mediates the hydrolysis of stearyl esters and is required for mobilization of stearyl ester, thereby playing a central role in lipid metabolism. This enzyme may have weak lipase activity toward triglycerides under some conditions.

The transgenic plant of this embodiment may comprise any polynucleotide encoding a sterol esterase. Preferably, the transgenic plant of this embodiment comprises a polynucleotide encoding a full-length polypeptide having sterol esterase activity, wherein the polypeptide has a sequence comprising amino acids 1 to 548 of SEQ ID NO:40.

### H. Succinate-CoA Ligase

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length succinate-CoA ligase polypeptide; wherein the transgenic plant demonstrates increased yield as compared to a wild type plant of the same variety which does not comprise the expression cassette.

As shown in Table 12 below, when transcription of the Synechocystis gene SLL1023 (SEQ ID NO:41) is targeted to plastids under control of the PcUbi promoter, transgenic plansts are larger than control plants, both under cycling drought and well-watered conditions. SLL1023 is a succinate-CoA ligase (EC 6.2.1.5). Succinate-CoA ligase catalyzes the reaction of GTP+succinate+CoA=GDP+phosphate+succinyl-CoA. This reaction is part of the TCA cycle for sugar metabolism.

The transgenic plant of this embodiment may comprise any polynucleotide encoding a succinate-CoA ligase. Preferably, the transgenic plant of this embodiment comprises a polynucleotide encoding a succinate-CoA ligase having a sequence comprising amino acids 1 to 401 of SEQ ID NO:42.

### I. Cobalt-precorrin-6A Reductase

In another embodiment, the invention provides a transgenic plant transformed with an expression cassette comprising, in operative association, an isolated polynucleotide encoding a promoter and an isolated polynucleotide encoding a full-length cobalt-precorrin-6A reductase polypeptide; wherein the transgenic plant demonstrates increased yield as compared to a wild type plant of the same variety which does not comprise the expression cassette.

As shown in Table 13 below, when the Synechocystis gene SLR0252 (SEQ ID NO:43) is expressed under control of the PcUbi promoter, transgenic plants were larger than control plants under cycling drought conditions. SLR0252 is a cobalt-precorrin-6A reductase (EC 1.3.1.54). Cobalt-precorrin-6A reductase catalyzes the reduction of the macrocycle of cobalt-precorrin-6X into cobalt-precorrin-6Y. Cobalt-precorrin-6Y is a co-factor of many enzymes.

The transgenic plant of this embodiment may comprise any polynucleotide encoding a cobalt-precorrin-6A reductase. Preferably, the transgenic plant of this embodiment comprises a polynucleotide encoding a full-length polypeptide encoding a cobalt-precorrin-6A reductase having a sequence comprising amino acids 1 to 261 of SEQ ID NO:44.

### J. Uroporphyrin-III C-methyltransferase

In another embodiment, the invention provides a transgenic plant transformed with an expression cassette comprising, in operative association, an isolated polynucleotide encoding a promoter capable of enhancing expression in leaves, and an isolated polynucleotide encoding a full-length uroporphyrin-III C-methyltransferase; wherein the transgenic plant demonstrates increased yield as compared to a wild type plant of the same variety which does not comprise the expression cassette. Table 14 below shows that when the E. coli gene b3803 (SEQ ID NO:45) is expressed under control of the USP promoter, transgenic plants were larger than control plants under well-watered growth conditions. The b3803 gene encodes a uroporphyrin-III C-methyltransferase, also known as S-adenosyl-L-methionine-dependent Uroporphyrinogen-III C-methyltransferase (SUMT), an enzyme involved in the biosynthesis of siroheme and cobalamin (vitamin B12). SUMT (EC 2.1.1.107) is a branchpoint enzyme that plays a key role in the biosynthesis of modified tetrapyrroles. By catalyzing the transformation of uroporphyrinogen III into precorrin-2, SUMT controls the flux to compounds such as vitamin B12 and siroheme, an important co-factor of nitrate reductase and sulfite reductase enzymes. In plants, uroporphyrinogen III enters the pathway that leads to chlorophyll synthesis.

The transgenic plant of this embodiment may preferably comprise any polynucleotide encoding a uroporphyrin-III C-methyltransferase. More preferably, the transgenic plant of this embodiment comprises a polynucleotide encoding a full-length polypeptide having uroporphyrin-III C-methyltransferase activity, wherein the polypeptide comprises a domain comprising amino acids 101 to 356 of SEQ ID NO:46; amino acids 97 to 353 of SEQ ID NO:48; amino acids 91 to 346 of SEQ ID NO:50; or amino acids 100 to 355 of SEQ ID NO:52. Most preferably, the transgenic plant of this embodiment comprises a polynucleotide encoding a uroporphyrin-III C-methyltransferase having a sequence comprising amino acids 1 to 393 of SEQ ID NO:46, amino acids 1 to 368 of SEQ ID NO:48; amino acids 1 to 363 of SEQ ID NO:50, or amino acids 1 to 379 of SEQ ID NO:52.

### K. Isoprenoid Biosynthesis Protein

In another embodiment, the invention provides a transgenic plant transformed with an expression cassette comprising, in operative association, an isolated polynucleotide encoding a promoter capable of enhancing gene expression in roots and shoots and an isolated polynucleotide encoding a full-length polypeptide having isoprenoid biosynthesis activity; wherein the transgenic plant demonstrates increased yield as compared to a wild type plant of the same variety which does not comprise the expression cassette.

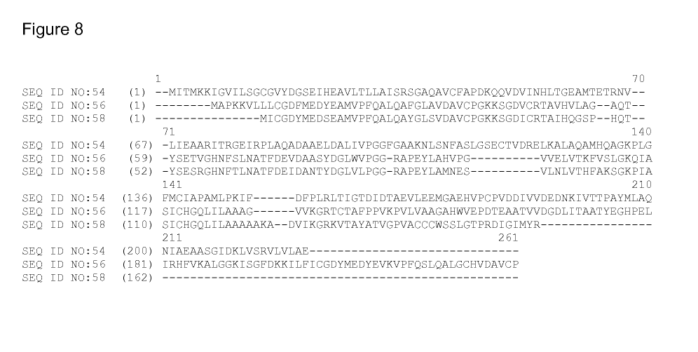

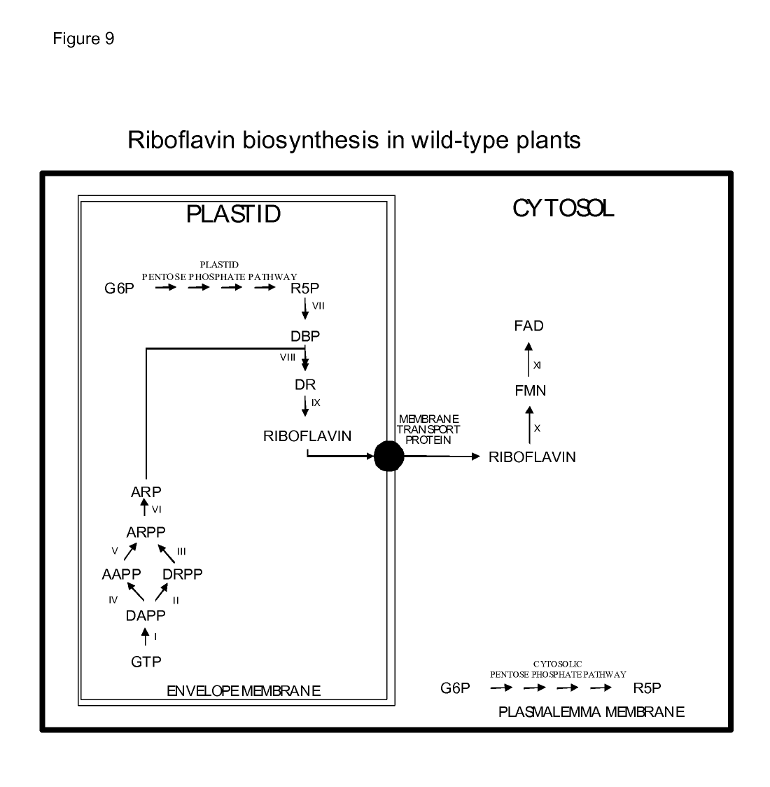

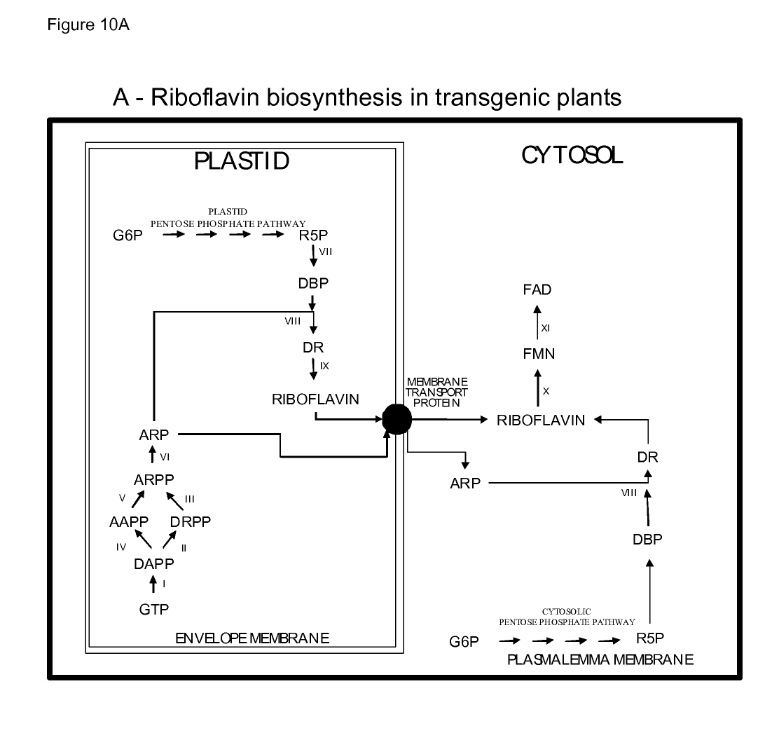

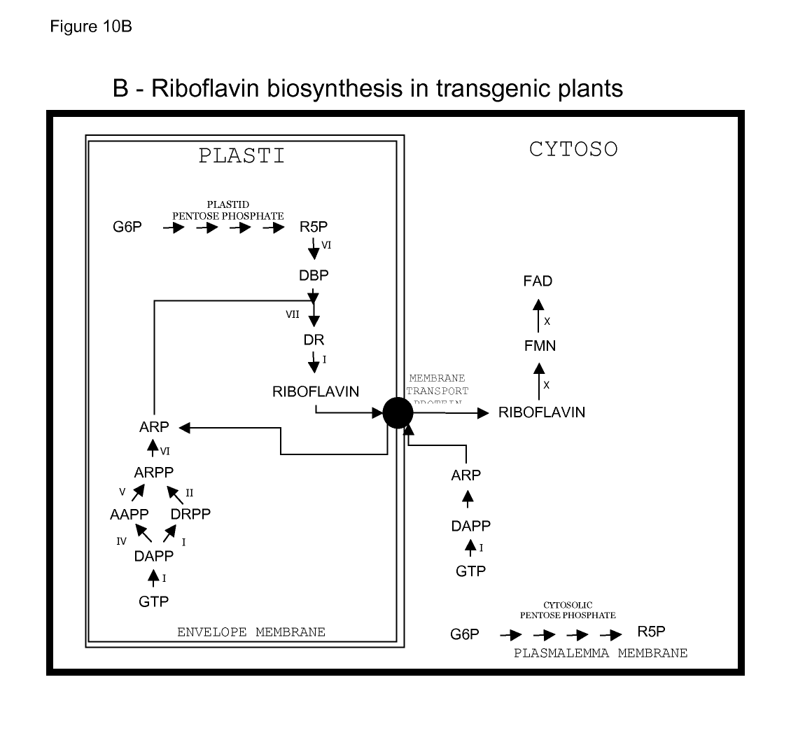

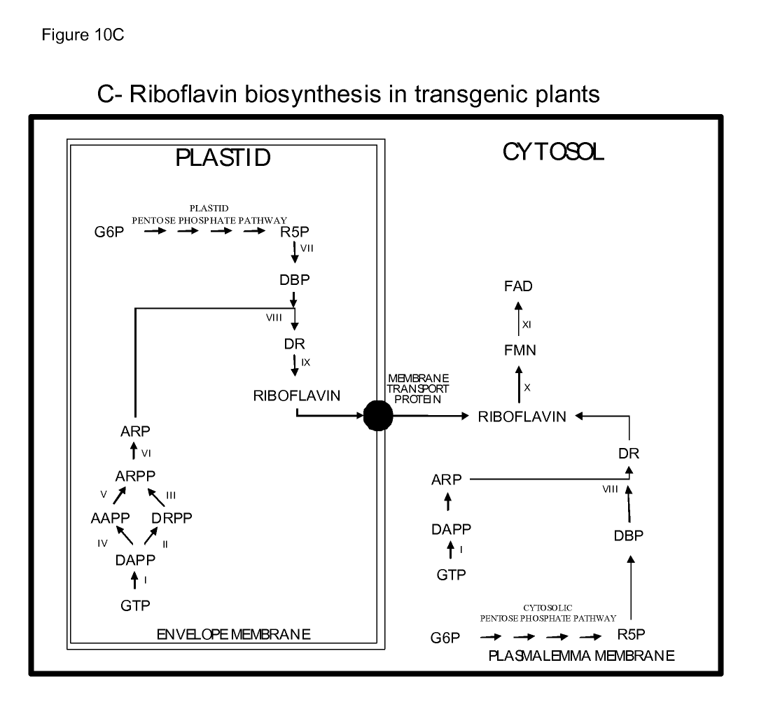

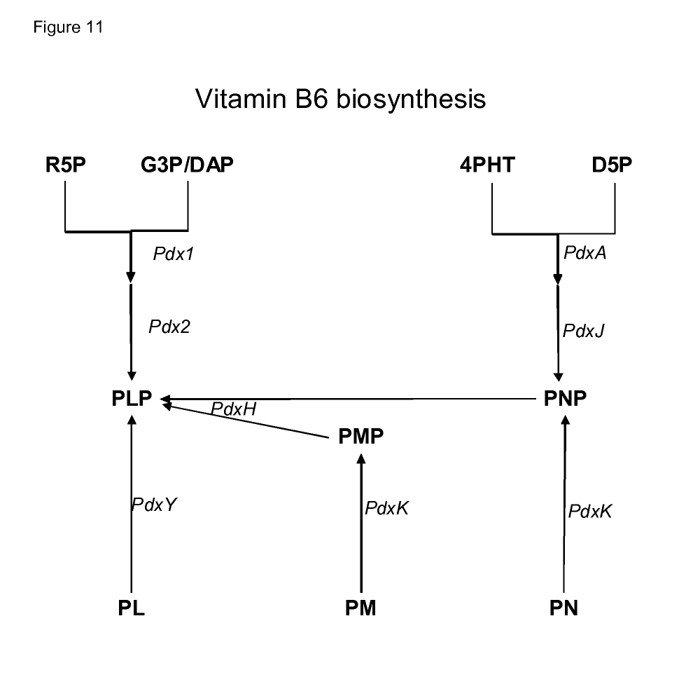

Table 15 below shows that when the E. coli gene b3209 (SEQ ID NO:53) is expressed under control of the Super promoter, transgenic plants are larger than control plants under well-watered growth conditions. While the specific function of the b3209 protein is not known, it has been shown to increase lycopene production in E. coli when over-expressed. Lycopene is an important component of the photosystem and a potent anti-oxidant that protects cells from oxidative damage. In plant cells it is also converted to other carotenoids and precursors for plant hormones. Isoprenoid biosynthesis proteins are characterized, in part, by the presence of a DJ-1_Pfp1 signature sequence. Such signature sequences are exemplified in the isoprenoid biosynthesis proteins set forth in FIG. 8.

The transgenic plant of this embodiment may comprise any polynucleotide encoding an isoprenoid biosynthesis protein. Preferably, the transgenic plant of this embodiment comprises a polynucleotide encoding a full-length polypeptide having isoprenoid biosynthesis activity, wherein the polypeptide comprises a DJ-1_Pfp1 signature sequence selected from the group consisting of amino acids 46 to 208 of SEQ ID NO:54; amino acids 32 to 189 of SEQ ID NO:56; and amino acids 25 to 151 of SEQ ID NO:58. Most preferably, the transgenic plant of this embodiment comprises a polynucleotide encoding an isoprenoid biosynthesis protein having a sequence comprising amino acids 1 to 220 of SEQ ID NO:54, amino acids 1 to 231 of SEQ ID NO:56, or amino acids 1 to 161 of SEQ ID NO:58.

### L. LysE Type Translocator

In another embodiment, the invention provides a transgenic plant transformed with an expression cassette comprising, in operative association, an isolated polynucleotide encoding a promoter capable of enhancing gene expression in roots and shoots and an isolated polynucleotide encoding a full-length polypeptide having LysE type translocator activity; wherein the transgenic plant demonstrates increased yield as compared to a wild type plant of the same variety which does not comprise the expression cassette.

As shown in Table 16 below, when the E. coli gene b2578 (SEQ ID NO:59) is expressed under control of the Super promoter, transgenic plants were larger than control plants under well-watered conditions. The b2578 gene encodes a LysE type translocator protein, a membrane protein with six predicted transmembrane domains, which is involved in maintaining intercellular levels of L-lysine by exporting excess L-lysine from the cell. These proteins are characterized, in part, by the presence of a LysE signature sequence.

The transgenic plant of this embodiment may comprise any polynucleotide encoding a LysE type translocator. Preferably, the transgenic plant of this embodiment comprises a polynucleotide encoding a full-length polypeptide having LysE type translocator activity, wherein the polypeptide comprises a LysE signature sequence comprising amino acids 14 to 195 of SEQ ID NO:60. Most preferably, the transgenic plant of this embodiment comprises a polynucleotide encoding a LysE type translocator having a sequence comprising amino acids 1 to 195 of SEQ ID NO:60.

### M. Branched-Chain Amino Acid Transporter

In another embodiment, the invention provides a transgenic plant transformed with an expression cassette comprising, in operative association, an isolated polynucleotide encoding a promoter capable of enhancing gene expression in roots and shoots; and an isolated polynucleotide encoding a full-length polypeptide having LIV-E family branched-chain amino acid transport activity; wherein the transgenic plant demonstrates increased yield as compared to a wild type plant of the same variety which does not comprise the expression cassette.

As shown in Table 17 below, when the E. coli gene b2682 (SEQ ID NO:61) is expressed under control of the Super promoter, transgenic plants were larger than control plants under well-watered growth conditions. The b2682 gene encodes a LIV-E family branched-chain amino acid transport protein, a membrane protein with five predicted transmembrane domains involved in the export of L-valine. These proteins are characterized, in part, by the presence of a AzIC signature sequence.

The transgenic plant of this embodiment may comprise any polynucleotide encoding a branched-chain amino acid transporter. Preferably, the transgenic plant of this embodiment comprises a polynucleotide encoding a full-length polypeptide having LIV-E family branched-chain amino acid transport activity, wherein the polypeptide comprises a AzIC signature sequence comprising amino acids 23 to 167 of SEQ ID NO:62. Most preferably, the transgenic plant of this embodiment comprises a polynucleotide encoding a branched-chain amino acid transporter having a sequence comprising amino acids 1 to 245 of SEQ ID NO:62.

### N. DNA-Binding Protein

In another embodiment, the invention provides a transgenic plant transformed with an expression cassette comprising, in operative association, an isolated polynucleotide encoding a promoter capable of enhancing gene expression in roots and shoots; and an isolated polynucleotide encoding a truncated DNA-binding polypeptide; wherein the transgenic plant demonstrates increased yield as compared to a wild type plant of the same variety which does not comprise the expression cassette.

As shown in Table 18 below, when the E. coli gene b3285 (SEQ ID NO:63) is expressed under control of the Super promoter, transgenic plants are larger than control plants under well-watered growth conditions. The b3285 gene encodes a truncated DNA-binding protein comprising the C-terminal portion of the E. coli SMF protein (public database accession number YP_{—}026211), and may facilitate the access of DNA-modifying proteins to genomic DNA. Such DNA-binding proteins are characterized, in part, by the presence of an SMF signature sequence.

The transgenic plant of this embodiment may comprise any polynucleotide homologous to the polynucleotide encoding the truncated DNA-binding protein of SEQ ID NO:64. Preferably, the transgenic plant of this embodiment comprises a polynucleotide encoding a DNA-binding protein having a sequence comprising amino acids 1 to 102 of SEQ ID NO:64.

### O. YscJ/FliF Protein

In another embodiment, the invention provides a transgenic plant transformed with an expression cassette comprising, in operative association, an isolated polynucleotide encoding a promoter capable of enhancing gene expression in roots and shoots; and an isolated polynucleotide encoding a full-length YscJ/FliF family polypeptide; wherein the transgenic plant demonstrates increased yield as compared to a wild type plant of the same variety which does not comprise the expression cassette.

As shown in Table 19 below, when the E. coli gene b1938 (SEQ ID NO:65), a YscJ/FliF family protein, is expressed under control of the Super promoter, transgenic pinats are larger than control plants. The YscJ/FliF family proteins stabilize membrane proteins and complexes such as transporters, which provide cells with important compounds for cell growth. YscJ/FliF family proteins are characterized, in part, by the presence of a YscJ_FliF signature sequence.

The transgenic plant of this embodiment may comprise any polynucleotide encoding an YscJ/FliF family protein. Preferably, the transgenic plant of this embodiment comprises a polynucleotide encoding a full-length polypeptide having YscJ/FliF activity, wherein the polypeptide comprises a first YscJ/FliF signature sequence comprising amino acids 17 to 225 of SEQ ID NO:66 and a second YscJ/FliF signature sequence comprising amino acids 250 to 429 of SEQ ID NO:66. Most preferably, the transgenic plant of this embodiment comprises a polynucleotide encoding a YscJ/FliF family protein having a sequence comprising amino acids 1 to 552 of SEQ ID NO:66.

### P. Riboflavin Biosynthetic Genes

In another embodiment, the invention provides a transgenic plant transformed with an expression cassette comprising, in operative association, a promoter and an isolated polynucleotide encoding a full length GTP cyclohydrolase II polypeptide which does not comprise a subcellular targeting sequence; wherein the transgenic plant demonstrates increased yield as compared to a wild type plant of the same variety that does not comprise the expression cassette.

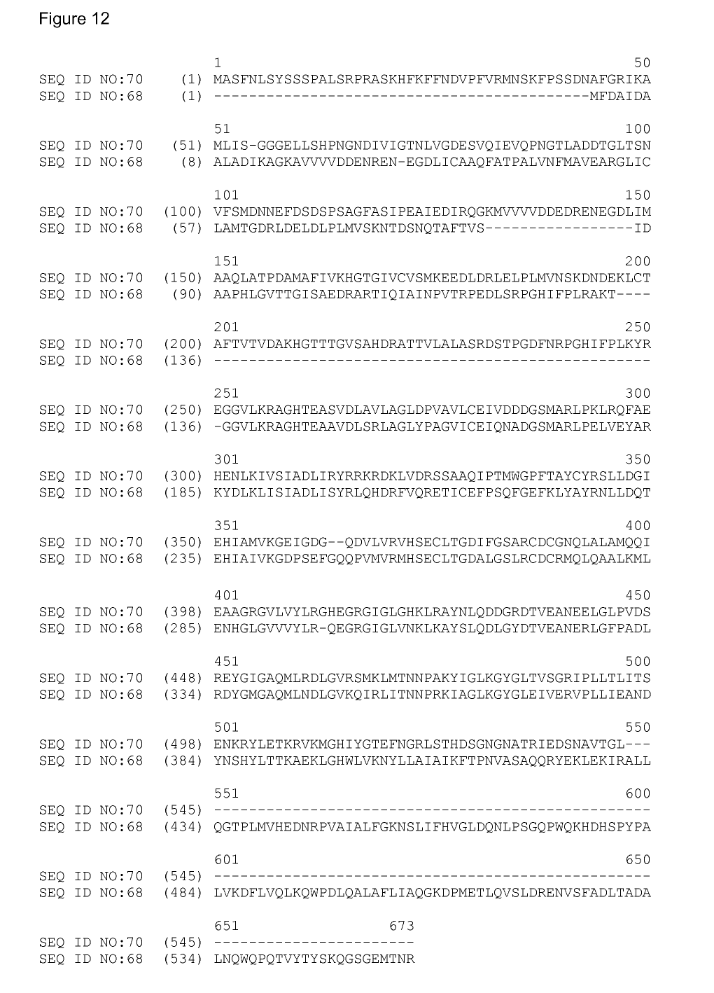

As shown in Table 20 below, when the Synechocystis gene SLL1894 (SEQ ID NO:67), a GTP cyclohydrolase II, was expressed under control of the PcUbi promoter with no subcellular targeting, transgenic plants were larger than control plants under water limited conditions. GTP cyclohydrase II enzymes are characterized, in part, by the presence of a DHBP_synthase signature sequence in the N-terminus and a GTP cyclohydrolase II signature sequence in the C-terminus. Such signature sequences are exemplified in the GTP cyclohydrolase II proteins set forth in FIG. 12.

The expression cassette employed in the transgenic plant of this embodiment may comprise any polynucleotide encoding a full-length polypeptide having GTP cyclohydrolase II activity which does not comprise a subcellular targeting peptide. Preferably, the GTP cyclohydrolase II polypeptide comprises a DHBP synthase domain selected from the groups consisting of amino acids 5 to 202 of SEQ ID NO:68 and amino acids 120 to 317 of SEQ ID NO:70 and a GTP cyclohydrolase II domain selected from the group consisting of amino acids 207 to 377 of SEQ ID NO:68 and amino acids 322 to 491 of SEQ ID NO:70. In accordance with the invention, when the G. max GTP cyclohydrolase II set forth in SEQ ID NO:70 is employed in the expression cassette, polynucleotide sequence encoding the subcellular targeting peptide (amino acids 1 to 49 of SEQ ID NO:70) is deleted. More preferably, the transgenic plant of this embodiment comprises a polynucleotide encoding a GTP cyclohydrolase II polypeptide having a sequence comprising amino acids 1 to 556 of SEQ ID NO:68 or amino acids 50 to 544 of SEQ ID NO:70.

In another embodiment, the invention provides a transgenic plant transformed with an expression cassette comprising, in operative association, an isolated polynucleotide encoding a promoter and an isolated polynucleotide encoding a lumazine synthase polypeptide which does not comprise a plastid targeting sequence; wherein the transgenic plant demonstrates increased yield as compared to a wild type plant of the same variety which does not comprise the expression cassette.

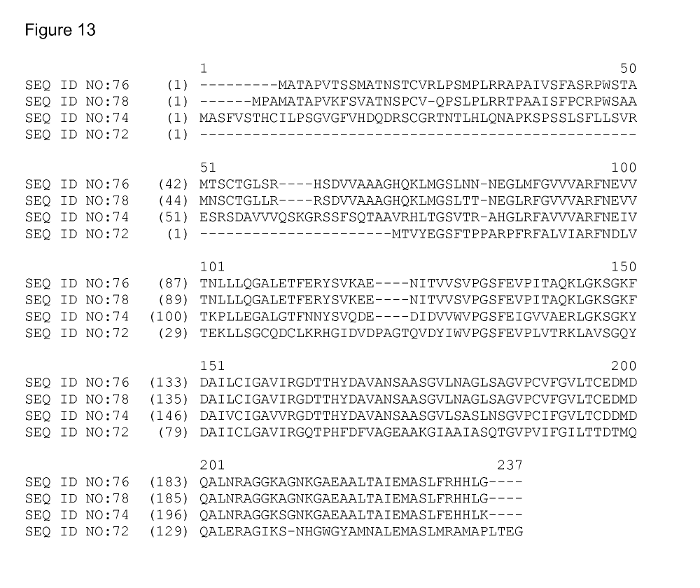

As shown in Tables 21 and 22 below, when the Synechocystis gene SLL1282 (SEQ ID NO:71), a lumazine synthase, is expressed under control of the PcUbi promoter, transgenic plants demonstrate increased yield as compared to a wild type plant of the same variety which does not comprise the SLL1282 gene. Lumazine synthase enzymes are characterized, in part, by the presence of a DMRL_synthase signature sequence. Such signature sequences are exemplified in the lumazine synthase proteins set forth in FIG. 13.

The expression cassette employed in the transgenic plant of this embodiment may comprise any polynucleotide encoding a lumazine synthase polypeptide which does not comprise a subcellular targeting peptide. Preferably, the transgenic plant of this embodiment comprises a polynucleotide encoding a full-length polypeptide having lumazine synthase activity, wherein the polypeptide comprises a DMRL_synthase signature sequence selected from the group consisting of amino acids 12 to 158 of SEQ ID NO:72; amino acids 83 to 226 of SEQ ID NO:74; amino acids 70 to 213 of SEQ ID NO:76, amino acids 70 to 215 of SEQ ID NO:78. In accordance with the invention, when any of the plant lumazine synthases set forth in SEQ ID NOs:100, 76, or 78 is employed in the expression cassette, the polynucleotide sequences encoding the subcellular targeting peptide (amino acids 1 to 53 of SEQ ID NO:74; amino acids 1 to 56 of SEQ ID NO:76, and amino acids 1 to 80 of SEQ ID NO:78) are deleted. More preferably, the transgenic plant of this embodiment comprises a polynucleotide encoding a lumazine synthase comprising amino acids 1 to 164 of SEQ ID NO:72, amino acids 54 to 228 of SEQ ID NO:74, amino acids 57 to 217 of SEQ ID NO:76, or amino acids 81 to 217 of SEQ ID NO:78.

### Q. Vitamin B6 Biosynthetic Genes

In another embodiment, the invention provides a transgenic plant transformed with an expression cassette comprising, in operative association, an isolated polynucleotide encoding a promoter capable of enhancing gene expression in roots and shoots and an isolated polynucleotide encoding a full length polypeptide which is capable of enhancing PLP synthesis. In this embodiment, the expression cassette may optionally further comprise an isolated polynucleotide encoding a mitochondrial or plastid transit peptide. Polynucleotides comprising any gene of the vitamin B6 synthetic pathway are suitable for use in this embodiment of the invention. For example, the vitamin B6 synthetic gene may be pdxY (e.g., SEQ ID NO:79), pdxH (e.g., SEQ ID NO:81), pdxK (e.g., SEQ ID NOs:83, 85, and 87), yfei (e.g., SEQ ID NOs: 89 and 91), Yn8fp (SEQ ID NO:93); pdxJ (e.g., SEQ ID NOs: 95 and 97), pdx1/ sor1 (e.g., SEQ IS NOs: 99, 101, 103, 105, 107, 109, 111, 113, 115, 117, or 119).

When the transgenic plant comprises the vitamin B6 biosynthetic gene PdxY, the promoter is preferably a root- or shoot-specific promoter and the expression cassette further comprises a polynucleotide encoding a plastid transit peptide. As shown in Tables 23 and 24 below, when the E. coli gene b1636 (SEQ ID NO:79), designated pdxY, is targeted to the plastid under control of the Super promoter, transgenic plants were larger than control plants under well watered and water-limited conditions.

The PdxY gene encodes a PL kinase which comprises a phosphomethyl-pyrimidine kinase domain. The transgenic plant of this embodiment may comprise any polynucleotide encoding a PL kinase. Preferably, the transgenic plant of this embodiment comprises a polynucleotide encoding a full-length polypeptide having PL kinase activity, wherein the polypeptide comprises a phosphomethyl-pyrimidine kinase signature sequence such as amino acids 61 to 259 of SEQ ID NO:80. More preferably, the transgenic plant of this embodiment comprises a polynucleotide encoding a PL kinase having a sequence comprising amino acids 1 to 287 of SEQ ID NO:80.

As shown in Table 25 below, when the Synechocystis gene SLL1779 (SEQ ID NO:95), designated pdxJ, is expressed under control of the PcUbi promoter, with or without subcellular targeting, transgenic plants are larger than control plants under well-watered conditions. Table 26 shows that when SLL1779 (SEQ ID NO:95) is targeted to mitochondria under control of the PcUbi promoter, transgenic plants are larger than control plants when tested under water-limited conditions. Accordingly, when the transgenic plant comprises the PdxJ gene, the expression cassette may optionally further comprise a polynucleotide encoding a mitochondrial or plastid transit peptide. As set forth above, the PdxJ enzyme acts in a concerted manner with the PdxA enzyme to form PNP. Amino acids 2 to 218 of SEQ ID NO:96 represent a signature sequence of the PdxJ gene from Synechocystis sp PCC6803. Preferably, the PdxJ gene employed in the expression cassette of this embodiment encodes a polypeptide amino acids 1 to 221 of SEQ ID NO:96 or amino acids 1 to 243 of SEQ ID NO:98.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO:3; SEQ ID NO:5; SEQ ID NO:9; SEQ ID NO:11; SEQ ID NO:13; SEQ ID NO:17; SEQ ID NO:19; SEQ ID NO:23; SEQ ID NO:25; SEQ ID NO:29; SEQ ID NO:31; SEQ ID NO:33; SEQ ID NO:37; SEQ ID NO:45; SEQ ID NO:53; SEQ ID NO:59; SEQ ID NO:61; SEQ ID NO:63; SEQ ID NO:65; SEQ ID NO:69; SEQ ID NO:73; SEQ ID NO:75; and SEQ ID NO:77. Also encompassed by the isolated polynucleotide of the invention is an isolated polynucleotide encoding a polypeptide having an amino acid sequence selected from the group consisting of SEQ ID NO:4; SEQ ID NO:6; SEQ ID NO:10; SEQ ID NO:12; SEQ ID NO:14; SEQ ID NO:18; SEQ ID NO:20; SEQ ID NO:24; SEQ ID NO:26; SEQ ID NO:30; SEQ ID NO:32; SEQ ID NO:34; SEQ ID NO:38; SEQ ID NO:54; SEQ ID NO:60; SEQ ID NO:62; SEQ ID NO:64; SEQ ID NO:70; SEQ ID NO:74; SEQ ID NO:76; and SEQ ID NO:78. A polynucleotide of the invention can be isolated using standard molecular biology techniques and the sequence information provided herein, for example, using an automated DNA synthesizer.

The isolated polynucleotides of the invention include homologs of the polynucleotides of Table 1. “Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide.

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

The invention further provides a recombinant expression vector which comprises an expression cassette selected from the group consisting of a) an expression cassette comprising, in operative association, an isolated polynucleotide encoding a promoter, an isolated polynucleotide encoding a subcellular targeting peptide, and an isolated polynucleotide encoding a full-length phosphatidate cytidylyltransferase polypeptide; b) an expression cassette comprising, in operative association, an isolated polynucleotide encoding a promoter capable of enhancing expression in leaves, an isolated polynucleotide encoding a mitochondrial transit peptide, and an isolated polynucleotide encoding an acyl-carrier protein; and c) an expression cassette comprising, in operative association, an isolated polynucleotide encoding a promoter, an isolated polynucleotide encoding a subcellular targeting peptide; and an isolated polynucleotide encoding an acyltransferase polypeptide.

In another embodiment, the recombinant expression vector of the invention comprises an isolated polynucleotide having a sequence selected from the group consisting of SEQ ID NO:4; SEQ ID NO:6; SEQ ID NO:10; SEQ ID NO:12; SEQ ID NO:14; SEQ ID NO:18; SEQ ID NO:20; SEQ ID NO:24; SEQ ID NO:26; SEQ ID NO:30; SEQ ID NO:32; SEQ ID NO:34; SEQ ID NO:38; SEQ ID NO:54; SEQ ID NO:60; SEQ ID NO:62; SEQ ID NO:64; SEQ ID NO:70; SEQ ID NO:74; SEQ ID NO:76; and SEQ ID NO:78. In addition, the recombinant expression vector of the invention comprises an isolated polynucleotide encoding a polypeptide having an amino acid sequence selected from the group consisting SEQ ID NO:4; SEQ ID NO:6; SEQ ID NO:10; SEQ ID NO:12; SEQ ID NO:14; SEQ ID NO:18; SEQ ID NO:20; SEQ ID NO:24; SEQ ID NO:26; SEQ ID NO:30; SEQ ID NO:32; SEQ ID NO:34; SEQ ID NO:38; SEQ ID NO:54; SEQ ID NO:60; SEQ ID NO:62; SEQ ID NO:64; SEQ ID NO:70; SEQ ID NO:74; SEQ ID NO:76; and SEQ ID NO:78.

The recombinant expression vector of the invention may also include one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (SEQ ID NO:123 or SEQ ID NO:124, Baeumlein et al. (1991) Mol. Gen. Genet. 225, 459-67), promoters of light-inducible genes such as ribulose-1.5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105, 357-67) and other leaf specific promoters such as those identified in Aleman, I. (2001) Isolation and characterization of leaf-specific promoters from alfalfa (Medicago sativa), Masters thesis, New Mexico State University, Los Cruces, N. Mex., and the like a constitutive promoter. Constitutive promoters are active under most conditions. Examples of constitutive promoters suitable for use in these embodiments include the parsley ubiquitin promoter from Petroselinum crispum described in WO 2003/102198 (SEQ ID NO:121); the CaMV 19S and 35S promoters, the sX CaMV 35S promoter, the Sep1 promoter, the rice actin promoter, the Arabidopsis actin promoter, the maize ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In other embodiments of the invention, a root or shoot specific promoter is employed. For example, the Super promoter (SEQ ID NO:122) provides high level expression in both root and shoots (Ni et al. (1995) Plant J. 7: 661-676). Other root specific promoters include, without limitation, the TobRB7 promoter (Yamamoto et al. (1991) Plant Cell 3, 371-382), the rolD promoter (Leach et al. (1991) Plant Science 79, 69-76); CaMV 35S Domain A (Benfey et al. (1989) Science 244, 174-181), and the like.

In accordance with the invention, a chloroplast transit sequence refers to a nucleotide sequence that encodes a chloroplast transit peptide. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); ferredoxin NADP+ oxidoreductase (Jansen et al. (1988) Curr. Genetics 13:517-522) (SEQ ID NO:13); nitrite reductase (Back et al (1988) MGG 212:20-26) and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

As defined herein, a mitochondrial transit sequence refers to a nucleotide sequence that encodes a mitochondrial presequence and directs the protein to mitochondria. Examples of mitochondrial presequences include groups consisting of ATPase subunits, ATP synthase subunits, Rieske-FeS protein, Hsp60, malate dehydrogenase, citrate synthase, aconitase, isocitrate dehydrogenase, pyruvate dehydrogenase, malic enzyme, glycine decarboxylase, serine hydroxymethyl transferase, isovaleryl-CoA dehydrogenase and superoxide dismutase. Such transit peptides are known in the art. See, for example, Von Heijne et al. (1991) Plant Mol. Biol. Rep. 9:104-126; Clark et al. (1989) J. Biol. Chem. 264:17544-17550; Romer et al. (1993) Biochem. Biophys. Res. Commun. 196:1414-1421; Faivre-Nitschke et al (2001) Eur J Biochem 268 1332-1339 and Shah et al. (1986) Science 233: 478-481.

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example any of the techniques described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression cassette described above, (b) regenerating a transgenic plant from the transformed plant cell; and selecting higher-yielding plants from the regenerated plant sells. The plant cell may be, but is not limited to, a protoplast, gamete producing cell, and a cell that regenerates into a whole plant. As used herein, the term “transgenic” refers to any plant, plant cell, callus, plant tissue, or plant part, that contains the expression cassette described above. In accordance with the invention, the expression casette is stably integrated into a chromosome or stable extra-chromosomal element, so that it is passed on to successive generations.

The effect of the genetic modification on plant growth and/or yield and/or stress tolerance can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, synthesis, evapotranspiration rates, general plant and/or crop yield, flowering, reproduction, seed setting, root growth, respiration rates, photosynthesis rates, metabolite composition, and the like.

The invention is further illustrated by the following examples, which are not to be construed in any way as imposing limitations upon the scope thereof.

### Example 1

### Characterization of Genes

YBR029C (SEQ ID NO:1), YKL192C (SEQ ID NO:7), YDR018C (SEQ ID NO:15), B2341 (SEQ ID NO:21), B0452 (SEQ ID NO:27), YNL202W (SEQ ID NO:35), YKL140W (SEQ ID NO:39), SLL1023 (SEQ ID NO:41), SLR0252 (SEQ ID NO:43), b3803 (SEQ ID NO:45), b3209 (SEQ ID NO:53), b2578 (SEQ ID NO:59), b2682 (SEQ ID NO:61), b3285 (SEQ ID NO:63), b1938 (SEQ ID NO:65), SLL1894 (SEQ ID NO:67), SLL1282 (SEQ ID NO:5), b1636 (SEQ ID NO:79) and SLR1779 (SEQ ID NO:95) were cloned using standard recombinant techniques. The functionality of each lead gene was predicted by comparing the amino acid sequence of the gene with other genes of known functionality. Homolog cDNAs were isolated from proprietary libraries of the respective species using known methods. Sequences were processed and annotated using bioinformatics analyses.

The YBR029C (SEQ ID NO:2) from S. cerevisiae encodes a phosphatidate cytidylyltransferase. The DNA sequence of this gene was blasted against proprietary databases of canola and maize cDNAs at an e value of e−10 (Altschul et al., 1997, Nucleic Acids Res. 25: 3389-3402). One homolog from canola and one homolog from maize were identified. The amino acid relatedness of these sequences is indicated in the alignments shown in FIG. 1.

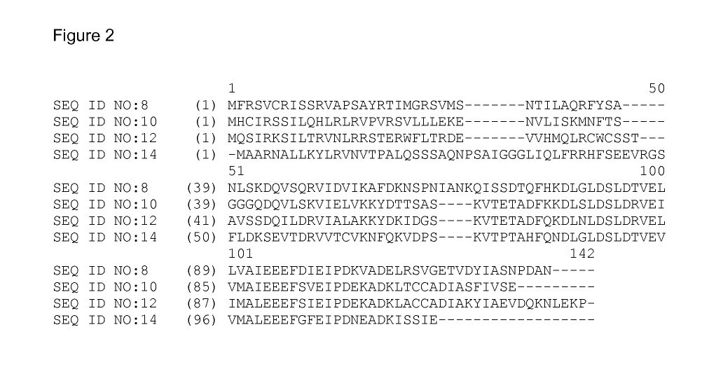

The YKL192C (SEQ ID NO:8) from S. cerevisiae encodes an acyl-carrier protein. The DNA sequence of this gene was blasted against proprietary databases of plant cDNAs at an e value of e−10 (Altschul et al., 1997, Nucleic Acids Res. 25: 3389-3402). One homolog each from canola, soybean, and sunflower were identified. The amino acid relatedness of these sequences is indicated in the alignments shown in FIG. 2.

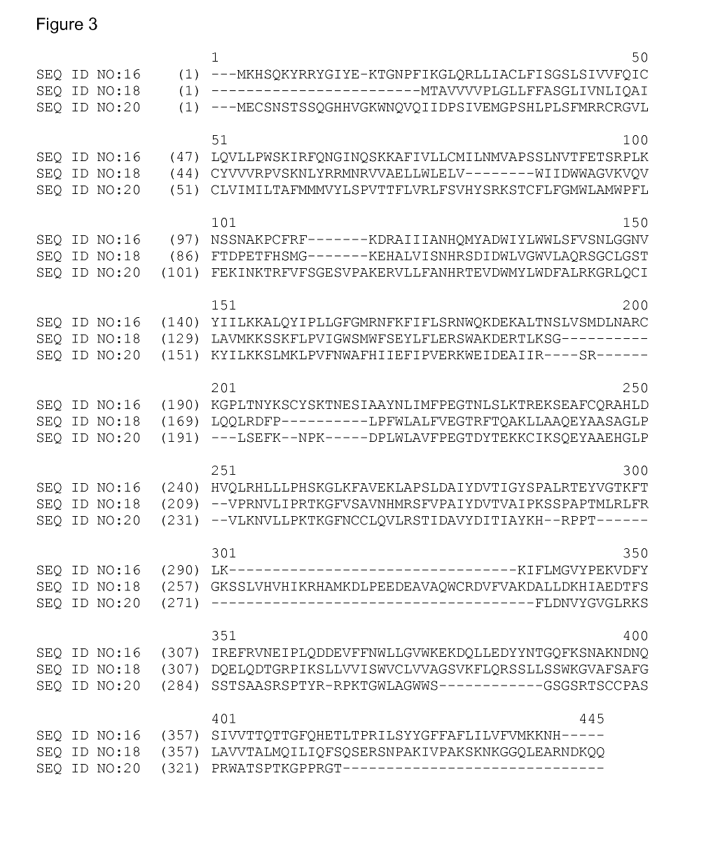

The of YDR018C (SEQ ID NO:16) from S. cerevisiae encodes an acyltransferase protein. The DNA sequence of this gene was blasted against proprietary databases of soybean and maize cDNAs at an e value of e−10 (Altschul et al., 1997, Nucleic Acids Res. 25: 3389-3402). One homolog from soybean and one from maize were identified. The amino acid relatedness of these sequences is shown in FIG. 3.

The B2341 (SEQ ID NO:21) gene from E. coli encodes a bifunctional anaerobic fatty acid oxidation complex polypeptide. The DNA sequence of this gene was blasted against a proprietary maize cDNA database at an e value of e−10 (Altschul et al., 1997, Nucleic Acids Res. 25: 3389-3402). Two homologs from maize were identified. The amino acid relatedness of these sequences is shown in FIG. 4.

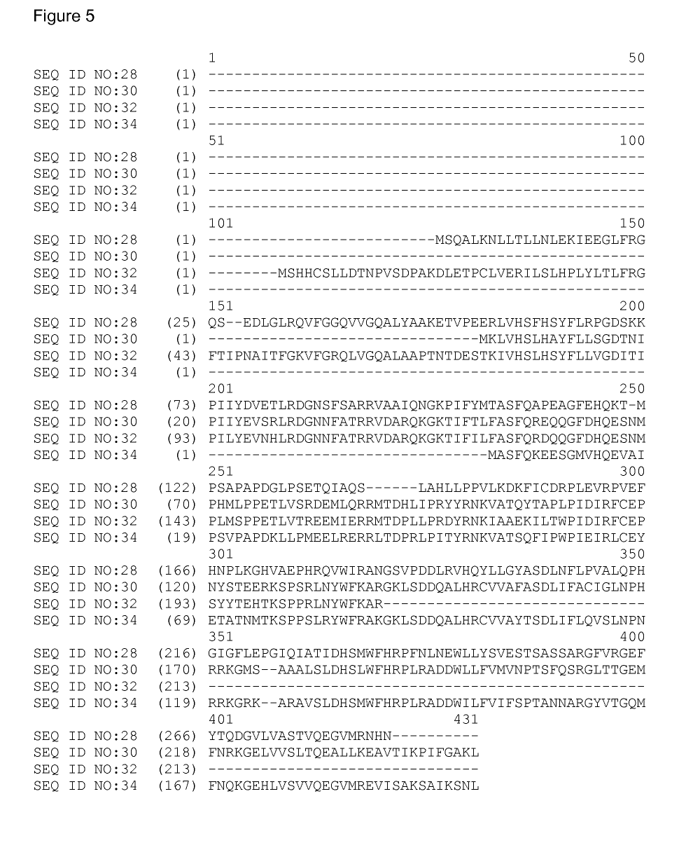

The B0452 (SEQ ID NO:28) from E. coli encodes an acyl-CoA thioesterase protein. The DNA sequence of this gene was blasted against proprietary databases of plant cDNAs at an e value of e−10 (Altschul et al., 1997, Nucleic Acids Res. 25: 3389-3402). Two homologs from canola and one from soybean were identified. The amino acid relatedness of these sequences is shown in FIG. 5.

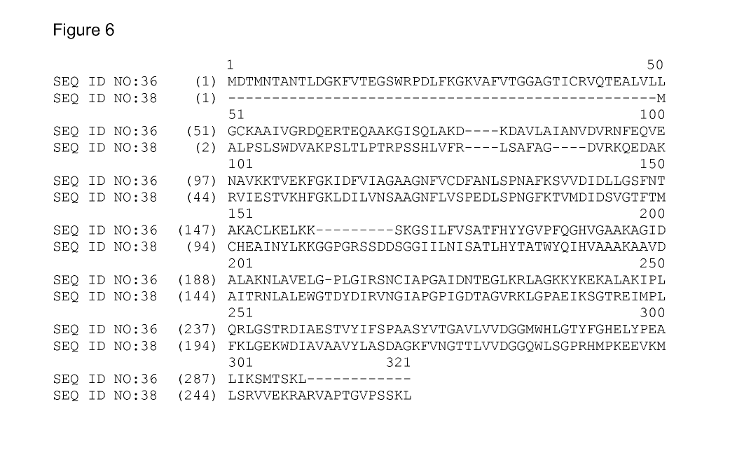

YNL202W (SEQ ID NO:36) from S. cerevisiae encodes a 2,4-dienoyl-CoA reductase protein. The DNA sequence of this gene was blasted against proprietary databases of plant cDNAs at an e value of e−10 (Altschul et al., 1997, Nucleic Acids Res. 25: 3389-3402). One homolog from sunflower were identified. The amino acid relatedness of these sequences is shown in FIG. 6.

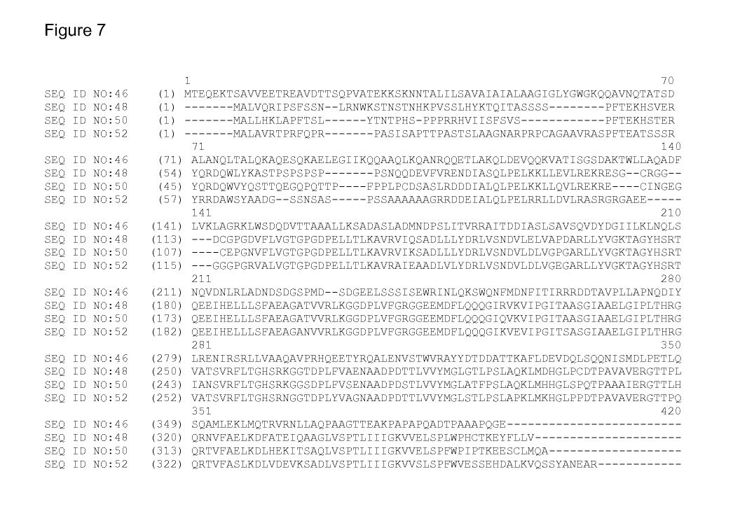

The b3803 gene from E. coli encodes a uroporphyrin-III C-methyltransferase. The full-length amino acid sequence of the functional homologs of b3803 from Table 11 were blasted against proprietary databases of cDNAs at an e value of e−10 (Altschul et al., 1997, Nucleic Acids Res. 25: 3389-3402). One homolog from Brassica napus, one homolog from Glycine max, and one homolog from Zea mays were identified. Homolog cDNAs were isolated from proprietary libraries of the respective species using known methods. Sequences were processed and annotated using bioinformatics analyses. The amino acid relatedness of these sequences is shown in FIG. 7.

The b3209 gene from E. coli encodes an isoprenoid biosynthesis protein. The full-length amino acid sequence of functional homologs of b3209 from Table 15 were blasted against proprietary databases of cDNAs at an e value of e−10 (Altschul et al., 1997, Nucleic Acids Res. 25: 3389-3402). One homolog from Glycine max and one homolog from Helianthus annuus were identified. The amino acid relatedness of these sequences is shown in FIG. 8.

The SLL1894 (SEQ ID NO:67) gene from Synechocystis sp. PCC 6803 encodes a GTP cyclohydrase II. The full-length DNA sequence of SLL1894 was blasted against a proprietary databases of soybean cDNAs at an e value of e−10 (Altschul et al., 1997, Nucleic Acids Res. 25: 3389-3402) and one homolog from soybean was identified. The amino acid relatedness of these sequences is shown in FIG. 12.

The SLL1282 gene (SEQ ID NO:71) from Synechocystis sp. PCC 6803 encodes a lumazine synthase. The full-length DNA sequence of this gene was blasted against proprietary databases of soybean and maize cDNAs at an e value of e−10 (Altschul et al., 1997, Nucleic Acids Res. 25: 3389-3402). One homolog from soybean and two homologs from maize were identified. The amino acid relatedness of these sequences is shown in FIG. 13.

### Example 2

### Overexpression of Lead Genes in Plants

Each of the genes described in Example 1 was ligated into an expression cassette using known methods. Four different promoters were used to control expression of the transgenes in Arabidopsis: the parsley ubiquitin promoter (SEQ ID NO:121) designated “PCUbi” in Tables 2 to 26; the super promoter (SEQ ID NO:122) designated “Super” in Tables 2 to 26; the USP promoter from Vicia faba (SEQ ID NO:124), designated “USP” in Tables 2 to 26 was used for expression of genes from prokaryotes or SEQ ID NO:123 was used for expression of genes from S. cerevisiae). For selective targeting of the polypeptides, the mitochondrial transit peptide from an A. thaliana gene encoding mitochondrial isovaleryl-CoA dehydrogenase designated “Mit” in Tables 2 to 26, SEQ ID NO:126 was used for expression of genes from prokaryotes or SEQ ID NO:128 was used for expression of genes from S. cerevisiae. In addition, for selective targeting of polypeptides to the chloroplast, the transit peptide of an Spinacia oleracea gene encoding ferredoxin nitrite reductase designated “Chlor” in Tables 2 to 26; SEQ ID NO:130 was used.

The Arabidopsis ecotype C24 was transformed with constructs containing the lead genes described in Example 1 using known methods. Seeds from T2 transformed plants were pooled on the basis of the promoter driving the expression, gene source species and type of targeting (chloroplastic, mitochondrial and cytoplasmic). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type Arabidopsis plants or to plants grown from a pool of randomly selected transgenic Arabidopsis seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various lead genes, due to different sites of DNA insertion and other factors that impact the level or pattern of gene expression.

Tables 2 to 26 show the comparison of measurements of the Arabidopsis plants. Percent change indicates the measurement of the transgenic relative to the control plants as a percentage of the control non-transgenic plants; p value is the statistical significance of the difference between transgenic and control plants based on a T-test comparison of all independent events where NS indicates not significant at the 5% level of probabilty; “No. of events” indicates the total number of independent transgenic events tested in the experiment; “No. of positive events” indicates the total number of independent transgenic events that were larger than the control in the experiment; “No. of negative events” indicates the total number of independent transgenic events that were smaller than the control in the experiment. NS indicates not significant at the 5% level of probability.

### A. Phosphatidate Cytidylyltransferase

The phosphatidate cytidylyltransferase designated as YBR029C (SEQ ID NO:2) was expressed in Arabidopsis using three constructs: in one construct YBR029C expression was controlled by the PCUbi promoter (SEQ ID NO:121) and targeted to chloroplasts; in another construct, YBR029C expression was controlled by the Super promoter (SEQ ID NO:122) and targeted to chloroplasts; and in the third construct YBR029C expression was controlled by the USP promoter (SEQ ID NO:123) and targeted to the mitochondria. Table 2 sets forth biomass and health index data obtained from the Arabidopsis plants transformed with these constructs and tested under water-limiting conditions.

TABLE 2

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| YBR029C | PCUbi | Chlor | Biomass at day | −16.7 | 0.0000 | 6 | 1 | 5 |
| | | | 20 | | | | | |
| YBR029C | PCUbi | Chlor | Biomass at day | −17.2 | 0.0000 | 6 | 0 | 6 |
| | | | 27 | | | | | |
| YBR029C | PCUbi | Chlor | Health index | −9.5 | 0.0056 | 6 | 1 | 5 |
| YBR029C | Super | Chlor | Biomass at day | 8.2 | 0.0340 | 6 | 4 | 2 |
| | | | 20 | | | | | |
| YBR029C | Super | Chlor | Biomass at day | 19.4 | 0.0000 | 6 | 5 | 1 |
| | | | 27 | | | | | |
| YBR029C | Super | Chlor | Health index | 0.6 | NS | 6 | 4 | 2 |
| YBR029C | USP | Mit | Biomass at day | −10.1 | 0.0040 | 8 | 2 | 6 |
| | | | 20 | | | | | |
| YBR029C | USP | Mit | Biomass at day | −9.5 | 0.0034 | 8 | 2 | 6 |
| | | | 27 | | | | | |
| YBR029C | USP | Mit | Health index | −1.8 | NS | 8 | 2 | 6 |

Table 2 shows that, under water limiting conditions, Arabidopsis plants expressing the YBR029C (SEQ ID NO:1) gene under control of the Super promoter with targeting of the protein product to the chloroplast and grown were significantly larger than the control plants. Table 2 also shows that the majority of independent Super promoter/chloroplast-targeted transgenic events were larger than the controls. Table 2 also shows that Arabidopsis plants grown under water limiting conditions and expressing the YBR029C gene under control of either the USP promoter with targeting of the protein product to mitochondria or the PCUbi promoter with targeting of the protein product to the chloroplast were significantly smaller than the control plants that did not express YBR029C. Table 2 also shows that the majority of independent transgenic events were smaller than the controls.

Table 3 sets forth biomass and health index data obtained from the Arabidopsis plants transformed with these constructs and tested under well watered conditions.

TABLE 3

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| YBR029C | PCUbi | Chlor | Biomass at day | 9.5 | 0.0001 | 6 | 5 | 1 |
| | | | 17 | | | | | |
| YBR029C | PCUbi | Chlor | Biomass at day | 5.5 | 0.0066 | 6 | 5 | 1 |
| | | | 21 | | | | | |
| YBR029C | PCUbi | Chlor | Health index | −9.6 | 0.0052 | 6 | 0 | 6 |
| YBR029C | Super | Chlor | Biomass at day | 5.2 | 0.050 | 6 | 4 | 2 |
| | | | 17 | | | | | |
| YBR029C | Super | Chlor | Biomass at day | 6.7 | 0.0035 | 6 | 4 | 2 |
| | | | 21 | | | | | |
| YBR029C | Super | Chlor | Health index | −10.7 | 0.0012 | 6 | 0 | 6 |
| YBR029C | USP | Chlor | Biomass at day | 6.2 | 0.0378 | 6 | 5 | 1 |
| | | | 17 | | | | | |
| YBR029C | USP | Chlor | Biomass at day | 6.2 | 0.0174 | 6 | 5 | 1 |
| | | | 21 | | | | | |
| YBR029C | USP | Chlor | Health index | −2.7 | NS | 6 | 2 | 4 |
| YBR029C | USP | Mit | Biomass at day | 14.7 | 0.0000 | 8 | 7 | 1 |
| | | | 17 | | | | | |
| YBR029C | USP | Mit | Biomass at day | 9.6 | 0.0000 | 8 | 8 | 0 |
| | | | 21 | | | | | |
| YBR029C | USP | Mit | Health index | 1.2 | NS | 8 | 4 | 4 |

Table 3 shows that, when grown under well-watered conditions, Arabidopsis plants expressing YBR029C were larger than the control plants that did not express YBR029C. The increase in biomass occurred with the PCUbi, Super and Ubi promoters and the effect of the transgene was observed in constructs where the YBR029C protein product (SEQ ID NO:2) was targeted to the mitochondria or the chloroplast.

### B. Acyl-Carrier Protein

The acyl-carrier protein designated as YKL192C (SEQ ID NO:8) was expressed in Arabidopsis using a construct wherein the acyl-carrier protein expression was controlled by the USP promoter (SEQ ID NO:123) and targeted to the mitochondria. Table 4 sets forth biomass and health index data obtained from the Arabidopsis plants transformed with these constructs and tested under water-limiting conditions.

TABLE 4

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| YKL192C | USP | Mit | Biomass at day | 29.8 | 0.0000 | 5 | 5 | 0 |
| | | | 20 | | | | | |
| YKL192C | USP | Mit | Biomass at day | 16.6 | 0.0000 | 5 | 4 | 1 |
| | | | 27 | | | | | |
| YKL192C | USP | Mit | Health index | 14.2 | 0.0000 | 5 | 4 | 1 |

Table 4 shows that Arabidopsis plants expressing the YKL192C (SEQ ID NO:7) gene under control of the USP promoter with targeting to the mitochondria that were grown under water limiting conditions were significantly larger than the control plants that did not express YKL192C. Table 4 also shows that the majority of independent transgenic events were larger and healthier than the controls.

### C. Acyltransferase

The acyltransferase designated as YDR018C (SEQ ID NO:16) was expressed in Arabidopsis using two different constructs: in one construct, YDR018C (SEQ ID NO:15) gene expression was controlled by the Super promoter (SEQ ID NO:122) and the YDR018C protein product was targeted to the choloroplast, and in the second construct, YDR018C expression was controlled by the USP promoter (SEQ ID NO:123) and targeted to the mitochondria. Table 5 sets forth biomass and health index data obtained from the Arabidopsis plants transformed with these constructs and tested under water-limiting conditions.

TABLE 5

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| YDR018C | Super | Chlor | Biomass at day | 6.8 | 0.0486 | 6 | 5 | 1 |
| | | | 20 | | | | | |
| YDR018C | Super | Chlor | Biomass at day | 21.1 | 0.0000 | 6 | 5 | 1 |
| | | | 27 | | | | | |
| YDR018C | Super | Chlor | Health index | 3.0 | NS | 6 | 4 | 2 |
| YDR018C | USP | Mit | Biomass at day | 11.5 | 0.0240 | 6 | 4 | 2 |
| | | | 20 | | | | | |
| YDR018C | USP | Mit | Biomass at day | 7.1 | NS | 6 | 4 | 2 |
| | | | 27 | | | | | |
| YDR018C | USP | Mit | Health index | 1.7 | NS | 6 | 4 | 2 |

Table 5 shows that Arabidopsis plants expressing YDR018C tended to be significantly larger than the control plants that did not express YDR018C when they were grown under water limiting conditions. Table 5 also shows that the majority of independent transgenic events were larger than the controls.

Table 6 sets forth biomass and health index data obtained from the Arabidopsis plants transformed with these constructs and tested under well watered conditions.

TABLE 6

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| YDR018C | Super | Chlor | Biomass at | −0.2 | NS | 6 | 3 | 3 |
| | | | day 17 | | | | | |
| YDR018C | Super | Chlor | Biomass at | 1.7 | NS | 6 | 3 | 3 |
| | | | day 21 | | | | | |
| YDR018C | Super | Chlor | Health index | −7.2 | 0.0375 | 6 | 0 | 6 |
| YDR018C | USP | Mit | Biomass at | 20.9 | 0.0000 | 6 | 6 | 0 |
| | | | day 17 | | | | | |
| YDR018C | USP | Mit | Biomass at | 20.3 | 0.0000 | 6 | 5 | 1 |
| | | | day 21 | | | | | |
| YDR018C | USP | Mit | Health index | 2.4 | NS | 6 | 3 | 3 |

Table 6 shows that Arabidopsis plants expressing YDR018C under control of the USP promoter with targeting to mitochondria were larger than the control plants that did not express YDR018C when grown under well watered conditions. However, when the YDR018C gene was expressed under control of the Super promoter and the protein product was targeted to the chloroplast, the plants were not significantly larger than control plants that did not express YDR018C, when the plants were grown under well watered conditions.

### D. Bifunctional Anaerobic Fatty Acid Oxidation Complex Polypeptide

The bifunctional anaerobic fatty acid oxidation complex polypeptide designated as B2341 (SEQ ID NO:22) was expressed in Arabidopsis using a construct wherein B2341 expression was controlled by the USP promoter (SEQ ID NO:124) and the protein product was targeted to the mitochondria. Table 7 sets forth biomass and health index data obtained from the Arabidopsis plants transformed with these constructs and tested under water-limiting conditions.

TABLE 7

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| b2341 | USP | Mit | Biomass at day | 24.2 | 0.0000 | 6 | 5 | 1 |
| | | | 20 | | | | | |
| b2341 | USP | Mit | Biomass at day | 15.9 | 0.0009 | 6 | 5 | 1 |
| | | | 27 | | | | | |
| b2341 | USP | Mit | Health index | 10.6 | 0.0007 | 6 | 5 | 1 |

Table 7 shows that Arabidopsis plants expressing the B2341 gene (SEQ ID NO:21) were significantly larger than the control plants that did not express B2341, when grown under water limiting conditions. Table 7 also shows that the majority of independent transgenic events were larger than the controls.

Table 8 sets forth biomass and health index data obtained from Arabidopsis plants transformed with these constructs and tested under well watered conditions.

TABLE 8

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| b2341 | USP | Mit | Biomass at | 8.5 | 0.0067 | 6 | 6 | 0 |
| | | | day 20 | | | | | |
| b2341 | USP | Mit | Biomass at | 1.6 | NS | 6 | 5 | 1 |
| | | | day 27 | | | | | |
| b2341 | USP | Mit | Health index | 14.2 | 0.0100 | 6 | 5 | 1 |

Table 8 shows that the majority of independent transgenic events expressing B2341 were larger than the control plants that did not express B2341 when grown under well-watered conditions. Table 8 also shows that the plants expressing B2341 were significantly darker green than the controls.

### E. Acyl-CoA Thioesterase

B0452 (SEQ ID NO:27) was expressed in Arabidopsis under control of the USP promoter (SEQ ID NO:124) and the protein product (SEQ ID NO:28) was targeted to the mitochondria. Table 9 sets forth biomass and health index data obtained from the Arabidopsis plants transformed with this construct and tested under cycling drought (CD) or well-watered (WW) conditions.

TABLE 9

| Assay | | | | | % | p- | No. of | Positive | Negative |
|---|---|---|---|---|---|---|---|---|---|
| Type | Gene | Promoter | Targeting | Measurement | Change | Value | Events | Events | Events |
| CD | B0452 | USP | Mit | Day 20 | 36.56 | 0.0000 | 4 | 4 | 0 |
| CD | B0452 | USP | Mit | Day 27 | 21.03 | 0.0000 | 4 | 4 | 0 |
| CD | B0452 | USP | Mit | Health | 15.14 | 0.0002 | 4 | 4 | 0 |
| | | | | Index | | | | | |
| WW | B0452 | USP | Mit | Day 17 | 30.57 | 0.0000 | 5 | 5 | 0 |
| WW | B0452 | USP | Mit | Day 21 | 15.39 | 0.0000 | 5 | 5 | 0 |
| WW | B0452 | USP | Mit | Health | 2.59 | NS | 5 | 3 | 2 |
| | | | | Index | | | | | |

Table 9 shows that transgenic plants expressing the B0452 gene under the control of the USP promoter with targeting to the mitochondria were significantly larger under either well-watered or drought conditions than the control plants that did not express the B0452 gene. The difference was even more striking at earlier stages, indicating a positive effect on seedling vigor. In these experiments, all of the independent transgenic events were larger than the controls in both the cycling drought and well-watered environments. The growth advantage of the transgenic plants was even more prominent under cycling drought conditions. The transgenic plants expressing the B0452 gene stayed significantly healthier than the wild-type control under the cycling drought conditions.

F. 2,4-dienoyl-CoA Reductase

YNL202W (SEQ ID NO:35) was expressed in Arabidopsis using two constructs, one of which transcription was under control of the USP promoter (SEQ ID NO:123) and the protein product YNL202W (SEQ ID NO:36) was targeted to the mitochondria, and the other construct in which transcriptional expression was under control of the PCUbi promoter (SEQ ID NO:121) and the protein product was targeted to the plastids, respectively. Table 10 sets forth biomass and health index data obtained from the Arabidopsis plants transformed with these constructs and tested under cycling drought (CD) and well-watered (WW) conditions.

TABLE 10

| Assay | | | | | % | p- | No. of | Positive | Negative |
|---|---|---|---|---|---|---|---|---|---|
| Type | Gene | Promoter | Targeting | Measurement | Change | Value | Events | Events | Events |
| CD | YNL202W | PCUbi | Chlor | Day 20 | −0.77 | 0.8773 | 6 | 3 | 3 |
| CD | YNL202W | PCUbi | Chlor | Day 27 | −13.24 | 0.0646 | 6 | 0 | 6 |
| CD | YNL202W | PCUbi | Chlor | Health | −0.78 | 0.8045 | 6 | 3 | 3 |
| | | | | Index | | | | | |
| CD | YNL202W | USP | Mit | Day 20 | 58.29 | 0.0000 | 6 | 6 | 0 |
| CD | YNL202W | USP | Mit | Day 27 | 27.33 | 0.0000 | 6 | 6 | 0 |
| CD | YNL202W | USP | Mit | Health | 0.36 | 0.9046 | 6 | 2 | 4 |
| | | | | Index | | | | | |
| WW1 | YNL202W | USP | Mit | Day 17 | 33.64 | 0.0000 | 8 | 8 | 0 |
| WW1 | YNL202W | USP | Mit | Day 21 | 25.52 | 0.0000 | 8 | 8 | 0 |
| WW1 | YNL202W | USP | Mit | Health | 0.16 | 0.9592 | 8 | 5 | 3 |
| | | | | Index | | | | | |
| WW2 | YNL202W | USP | Mit | Day 17 | 31.51 | 0.0000 | 6 | 6 | 0 |
| WW2 | YNL202W | USP | Mit | Day 21 | 21.82 | 0.0000 | 6 | 6 | 0 |
| WW2 | YNL202W | USP | Mit | Health | 12.62 | 0.0002 | 6 | 5 | 1 |
| | | | | Index | | | | | |

Table 10 shows that transgenic plants expressing the YNL202W gene under control of the USP promoter with targeting to the mitochondria were significantly larger under cycling drought conditions and in two experiments (WW1 and WW2) under well-watered conditions than the control plants that did not express the YNL202W gene. In these experiments, all the independent transgenic events with mitochondria targeting were larger than the controls in both the cycling drought and well-watered environments. The transgenic plants expressing the YNL202W gene under control of the USP promoter with targeting to the mitochondria were also significantly healthier in one experiment under well-watered conditions than the control plants that did not express the YNL202W gene.

Table 10 shows that transgenic plants expressing the YNL202W gene under control of the PCUbi promoter with targeting to the plastids were smaller but not significantly different compared to the control plants that did not express the YNL202W gene, under cycling drought conditions.

### G. Sterol Esterase

YKL140W (SEQ ID NO:39) was expressed in Arabidopsis using three different constructs: in one construct, expression was controlled by the PCUbi promoter (SEQ ID NO:121) and the protein product (SEQ ID NO:40) was targeted to the mitochondria, expression in the second construct was also under the control of the PCUbi promoter, but the protein product was targeted to plastids; and expression in the third construct was controlled by the USP promoter (SEQ ID NO:123) with the protein product being targeted to plastids. Table 11 sets forth biomass and health index data obtained from the Arabidopsis plants transformed with these constructs and tested under cycling drought or well-watered conditions.

TABLE 11

| Assay | | | | | % | | No. of | Positive | Negative |
|---|---|---|---|---|---|---|---|---|---|
| Type | Gene | Promoter | Targeting | Measurement | Change | p-Value | Events | Events | Events |
| CD | YKL140W | USP | Mit | Day 20 | 13.86 | 0.0003 | 6 | 6 | 0 |
| CD | YKL140W | USP | Mit | Day 27 | −2.03 | 0.5931 | 6 | 3 | 3 |
| CD | YKL140W | USP | Mit | Health | 5.65 | 0.0969 | 6 | 5 | 1 |
| | | | | Index | | | | | |
| CD | YKL140W | USP | Chlor | Day 20 | −37.76 | 0.0000 | 5 | 0 | 5 |
| CD | YKL140W | USP | Chlor | Day 27 | −21.06 | 0.0000 | 5 | 0 | 5 |
| CD | YKL140W | USP | Chlor | Health | −10.81 | 0.0001 | 5 | 0 | 5 |
| | | | | Index | | | | | |
| WW | YKL140W | PCUbi | Chlor | Day 17 | 42.97 | 0.0000 | 5 | 5 | 0 |
| WW | YKL140W | PCUbi | Chlor | Day 21 | 26.82 | 0.0000 | 5 | 5 | 0 |
| WW | YKL140W | PCUbi | Chlor | Health | −1.09 | 0.7119 | 5 | 2 | 3 |
| | | | | Index | | | | | |

Table 11 shows that transgenic plants expressing the YKL140W gene under control of the PCUbi promoter with targeting to the plastids were significantly larger under well-watered conditions than the control plants that did not express the YKL140W gene. In these experiments, all of the independent transgenic events with plastid targeting were larger than the controls in the cycling drought environment. Transgenic plants expressing the YKL140W gene under control of the USP promoter with targeting to the mitochondria were also significantly larger under cycling drought conditions than the control wild-type plants. Table 11 shows that transgenic plants expressing the YKL140W gene under control of the USP promoter with targeting to the plastid were significantly smaller under drought conditions than the control plants that did not express the YKL140W gene. Additionally, these transgenic plants had lower health index scores relative to the control in water-limited conditions.

### H. Succinate-CoA Ligase

The Synechocystis gene SLL1023 (SEQ ID NO:41) was expressed in Arabidopsis under control of the PCUbi promoter (SEQ ID NO:121) and targeted to the plastids. Table 12 sets forth biomass and health index data obtained from Arabidopsis plants transformed with this construct and tested under cycling drought and well-watered conditions.

TABLE 12

| Assay | | | | | % | p- | No. of | Positive | Negative |
|---|---|---|---|---|---|---|---|---|---|
| Type | Gene | Promoter | Targeting | Measurement | Change | Value | Events | Events | Events |
| CD | SLL1023 | PCUbi | Chlor | Day 20 | 43.02 | 0.0000 | 6 | 6 | 0 |
| CD | SLL1023 | PCUbi | Chlor | Day 27 | 37.78 | 0.0000 | 6 | 6 | 0 |
| CD | SLL1023 | PCUbi | Chlor | Health | 0.61 | 0.8357 | 6 | 3 | 3 |
| | | | | Index | | | | | |
| WW | SLL1023 | PCUbi | Chlor | Day 17 | 24.08 | 0.0000 | 6 | 6 | 0 |
| WW | SLL1023 | PCUbi | Chlor | Day 21 | 16.79 | 0.0000 | 6 | 6 | 0 |
| WW | SLL1023 | PCUbi | Chlor | Health | 9.33 | 0.0038 | 6 | 5 | 1 |
| | | | | Index | | | | | |

Table 12 shows that transgenic plants expressing the SLL1023 gene under control of the PCUbi promoter with targeting to the plastids were significantly larger under cycling drought and well-watered conditions than the control plants that did not express the SLL1023 gene. In these experiments, all the independent transgenic events with plastid targeting were larger than the controls under both cycling drought and well-watered conditions. The transgenic plants expressing the SLL1023 gene under control of the PCUbi promoter with targeting to the plastids were also significantly healthier under well-watered conditions than the control plants. The presence of the SLL1023 protein in the plastids promoted plant growth under both well-watered and drought conditions.

### I. Cobalt-precorrin-6A Reductase

The Synechocystis gene SLR0252 (SEQ ID NO:43) was expressed in Arabidopsis under control of the PCUbi promoter (SEQ ID NO:121). Table 13 sets forth biomass and health index data obtained from the Arabidopsis plants transformed with this construct and tested under cycling drought conditions.

TABLE 13

| Assay | | | | | % | p- | No. of | Positive | Negative |
|---|---|---|---|---|---|---|---|---|---|
| Type | Gene | Promoter | Targeting | Measurement | Change | Value | Events | Events | Events |
| CD | SLR0252 | PCUbi | None | Day 20 | 20.20 | 0.0000 | 5 | 5 | 0 |
| CD | SLR0252 | PCUbi | None | Day 27 | 16.30 | 0.0001 | 5 | 5 | 0 |
| CD | SLR0252 | PCUbi | None | Health | −4.07 | 0.0076 | 5 | 0 | 5 |
| | | | | Index | | | | | |

Table 13 shows that transgenic plants expressing the SLR0252 gene under control of the PCUbi promoter were significantly larger under cycling drought conditions than the control plants that did not express the SLR0252 gene. In these experiments, all the independent transgenic events were larger than the controls in the cycling drought environment. As evidenced by the observation that the transgenic plants were larger than the control under cycling drought conditions, the presence of the SLR0252 protein in the cytoplasm enabled the transgenic plant to growth better under water-limited conditions.

### J. Uroporphyrin-III C-methyltransferase

The E. coli gene designated b3803 (SEQ ID NO:45), encoding a uroporphyrin-III C-methyltransferase, was expressed in Arabidopsis using two different constructs: constructs controlled by the USP promoter (SEQ ID NO:124) and constructs controlled by the super promoter (SEQ ID NO:122). Table 14 sets forth biomass data obtained from Arabidopsis plants transformed with these constructs and tested under well-watered conditions.

TABLE 14

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b3803 | USP | Biomass at | wt | 15.31 | 0.0000 | 6 | 6 | 0 |
| | | day 20 | | | | | | |
| b3803 | Super | Biomass at | wt | −1.87 | 0.5346 | 6 | 3 | 3 |
| | | day 20 | | | | | | |
| b3803 | Super | Biomass at | wt | −1.25 | 0.6151 | 6 | 3 | 3 |
| | | day 27 | | | | | | |
| b3803 | USP | Biomass at | wt | 14.86 | 0.0000 | 6 | 6 | 0 |
| | | day 27 | | | | | | |
| b3803 | Super | Biomass at | superpool | 8.89 | 0.0139 | 6 | 5 | 1 |
| | | day 20 | | | | | | |
| b3803 | Super | Biomass at | superpool | 2.22 | 0.4364 | 6 | 3 | 3 |
| | | day 27 | | | | | | |

Table 14 shows that Arabidopsis plants expressing b3803 under control of the USP promoter that were grown under well-watered conditions were significantly larger than the control plants that did not express b3803. Table 14 also shows that the majority of independent transgenic events were larger than the controls.

### K. Isoprenoid Biosynthesis Protein

The gene designated b3209 (SEQ ID NO:53), endcoding an isoprenoid biosynthesis protein, was expressed in Arabidopsis using a construct controlled by the super promoter (SEQ ID NO:122). Table 15 sets forth biomass data obtained from Arabidopsis plants transformed with these constructs and tested under well-watered conditions.

TABLE 15

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b3209 | Super | Biomass at | wt | 17.71 | 0.0000 | 7 | 7 | 0 |
| | | day 20 | | | | | | |
| b3209 | Super | Biomass at | wt | 14.47 | 0.0000 | 7 | 7 | 0 |
| | | day 27 | | | | | | |
| b3209 | Super | Biomass at | superpool | 29.46 | 0.0000 | 7 | 7 | 0 |
| | | day 20 | | | | | | |
| b3209 | Super | Biomass at | superpool | 22.10 | 0.0000 | 7 | 7 | 0 |
| | | day 27 | | | | | | |

Table 15 shows that Arabidopsis plants expressing b3209 under control of the super promoter that were grown under well-watered conditions were significantly larger than the control plants that did not express b3209. Table 15 also shows that the majority of independent transgenic events were larger than the controls.

### L. LysE Type Translocator

The E. coli gene designated b2578 (SEQ ID NO:59), encoding a LysE type translocator, was expressed in Arabidopsis using a construct controlled by the super promoter (SEQ ID NO:122). Table 16 sets forth biomass and health index data obtained from Arabidopsis plants transformed with these constructs and tested under well-watered conditions.

TABLE 16

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b2578 | Super | Health index | wt | −6.74 | 0.0221 | 7 | 1 | 6 |
| b2578 | Super | Biomass at | wt | 7.66 | 0.0352 | 7 | 5 | 2 |
| | | day 20 | | | | | | |
| b2578 | Super | Biomass at | wt | 8.22 | 0.0061 | 7 | 6 | 1 |
| | | day 27 | | | | | | |
| b2578 | Super | Health index | superpool | 8.17 | 0.0200 | 7 | 6 | 1 |
| b2578 | Super | Biomass at | superpool | 18.41 | 0.0000 | 7 | 6 | 1 |
| | | day 20 | | | | | | |
| b2578 | Super | Biomass at | superpool | 15.44 | 0.0000 | 7 | 6 | 1 |
| | | day 27 | | | | | | |

Table 16 shows that Arabidopsis plants expressing b2578 that were grown under well-watered conditions were significantly larger than the control plants that did not express b2578. Table 16 also shows that the majority of independent transgenic events were larger than the controls.

### M. Branched-Chain Amino Acid Transporter

The gene designated b2682 (SEQ ID NO:61), encoding a branched-chain amino acid transporter, was expressed in Arabidopsis using a construct controlled by the super promoter (SEQ ID NO:122). Table 17 sets forth biomass and health index data obtained from the Arabidopsis plants transformed with these constructs and tested under well-watered conditions.

TABLE 17

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b2682 | Super | Health index | wt | −0.95 | 0.7523 | 7 | 3 | 4 |
| b2682 | Super | Biomass at | wt | 16.28 | 0.0000 | 7 | 7 | 0 |
| | | day 20 | | | | | | |
| b2682 | Super | Biomass at | wt | 7.49 | 0.0073 | 7 | 7 | 0 |
| | | day 27 | | | | | | |
| b2682 | Super | Health index | superpool | 14.89 | 0.0000 | 7 | 6 | 1 |
| b2682 | Super | Biomass at | superpool | 27.89 | 0.0000 | 7 | 7 | 0 |
| | | day 20 | | | | | | |
| b2682 | Super | Biomass at | superpool | 14.66 | 0.0000 | 7 | 7 | 0 |
| | | day 27 | | | | | | |

Table 17 shows that Arabidopsis plants expressing b2682 under control of the super promoter that were grown under well-watered conditions were significantly larger than the control plants that did not express b2682. Table 17 also shows that the majority of independent transgenic events were larger than the controls.

### N. DNA-Binding Protein

The gene designated b3285 (SEQ ID NO:63), encoding a DNA-binding protein, was expressed in Arabidopsis using a construct controlled by the super promoter (SEQ ID NO:122). Table 18 sets forth biomass and health index data obtained from Arabidopsis plants transformed with these constructs and tested under well-watered conditions.

TABLE 18

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b3285 | Super | Health index | wt | −6.46 | 0.0351 | 7 | 2 | 5 |
| b3285 | Super | Biomass at | wt | 16.84 | 0.0000 | 7 | 6 | 1 |
| | | day 20 | | | | | | |
| b3285 | Super | Biomass at | wt | 14.41 | 0.0000 | 7 | 7 | 0 |
| | | day 27 | | | | | | |
| b3285 | Super | Health index | superpool | 8.49 | 0.0200 | 7 | 5 | 2 |
| b3285 | Super | Biomass at | superpool | 28.51 | 0.0000 | 7 | 7 | 0 |
| | | day 20 | | | | | | |
| b3285 | Super | Biomass at | superpool | 21.75 | 0.0000 | 7 | 7 | 0 |
| | | day 27 | | | | | | |

Table 18 shows that Arabidopsis plants expressing b3285 under control of the super promoter that were grown under well-watered conditions were significantly larger than the control plants that did not express b3285. Table 18 also shows that the majority of independent transgenic events were larger than the controls.

### O. YscJ/FliF Protein

The gene designated b1938 (SEQ ID NO:65), encoding a YscJ/FliF protein, was expressed in Arabidopsis using a construct controlled by the super promoter (SEQ ID NO:122). Table 19 sets forth biomass and health index data obtained from the Arabidopsis plants transformed with these constructs and tested under well-watered conditions.

TABLE 19

| | | | | | | | No of | No. of |
|---|---|---|---|---|---|---|---|---|
| | | | Control | % | p- | No. of | Positive | Negative |
| Gene | Promoter | Measurement | type | Change | Value | Events | Events | Events |
| b1938 | Super | Biomass at | wt | 5.64 | 0.1074 | 7 | 5 | 2 |
| | | day 20 | | | | | | |
| b1938 | Super | Biomass at | wt | 7.41 | 0.0123 | 7 | 5 | 2 |
| | | day 27 | | | | | | |
| b1938 | Super | Biomass at | superpool | 16.18 | 0.0000 | 7 | 6 | 1 |
| | | day 20 | | | | | | |
| b1938 | Super | Biomass at | superpool | 14.57 | 0.0000 | 7 | 5 | 2 |
| | | day 27 | | | | | | |

Table 19 shows that Arabidopsis plants expressing b1938 under control of the super promoter that were grown under well-watered conditions were significantly larger than the control plants that did not express b1938. Table 19 also shows that the majority of independent transgenic events were larger than the controls.

### P. Riboflavin Biosynthetic Genes

The Synechocystis gene designated SLL1894 (SEQ ID NO:67) encoding GTP cyclohydrolase II was expressed in Arabidopsis using three different constructs controlled by the parsley ubiquitin promoter (SEQ ID NO:121): constructs with no subcellular targeting, constructs targeted to the chloroplast, and constructs targeted to mitochondria. Table 20 sets forth biomass and health index data obtained from Arabidopsis plants transformed with SLL1894 under control of the parsley ubiquitin promoter with and without subcellular targeting, and tested under water limited conditions.

TABLE 20

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| sll1894 | PCUbi | None | Biomass at | 12.2 | 0.0016 | 7 | 6 | 1 |
| | | | 20 days | | | | | |
| sll1894 | PCUbi | None | Biomass at | 7.8 | 0.0499 | 7 | 5 | 2 |
| | | | 27 days | | | | | |
| sll1894 | PCUbi | None | Health Index | 9.4 | 0.0092 | 7 | 5 | 2 |
| sll1894 | PCUbi | Mito | Biomass at | −2.1 | NS | 6 | 3 | 3 |
| | | | 20 days | | | | | |
| sll1894 | PCUbi | Mito | Biomass at | −21.8 | 0.0002 | 6 | 0 | 6 |
| | | | 27 days | | | | | |
| sll1894 | PCUbi | Mito | Health Index | 2.5 | NS | 6 | 5 | 1 |
| sll1894 | PCUbi | Chlor | Biomass at | −1.3 | NS | 6 | 3 | 3 |
| | | | 20 days | | | | | |
| sll1894 | PCUbi | Chlor | Biomass at | −0.9 | NS | 6 | 4 | 2 |
| | | | 27 days | | | | | |
| sll1894 | PCUbi | Chlor | Health Index | 7.1 | 0.0327 | 6 | 5 | 1 |

Transgenic plants expressing the SLL1894 gene with no subcellular targeting were significantly larger under water limited conditions than the control plants that did not express the SLL1894 gene. In addition, the transgenic plants were darker green in color than the controls under water limited conditions as shown by the increased health index. In these experiments, the majority of the independent transgenic events were larger than the controls in the water limited environment.

Transgenic plants expressing the SLL1894 gene with subcellular targeting to the mitochondria were significantly smaller under water limited conditions than the control plants that did not express the SLL1894 gene. Transgenic plants expressing the SLL1894 gene with subcellular targeting to the plastid were similar in size under water limited conditions to the control plants that did not express the SLL1894 gene, but had a larger health index.

The Synechocystis gene designated gene SLL1282 (SEQ ID NO:71) encoding lumazine synthase was expressed in Arabidopsis using a construct controlled by the parsley ubiquitin promoter (SEQ ID NO:121) with no subcellular targeting. Table 21 sets forth biomass and health index data obtained from Arabidopsis plants transformed with this construct and tested under well-watered conditions.

TABLE 21

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| sll1282 | PCUbi | None | Biomass at | 2.2 | NS | 6 | 3 | 3 |
| | | | 17 days | | | | | |
| sll1282 | PCUbi | None | Biomass at | −0.6 | NS | 6 | 2 | 4 |
| | | | 21 days | | | | | |
| sll1282 | PCUbi | None | Health Index | −1.5 | NS | 6 | 3 | 3 |

The growth of the Arabidopsis plants expressing the SLL1282 gene controlled by the PCUbi promoter and with no subcellular targeting was similar to control plants under well watered conditions.

Table 22 sets forth biomass and health index data obtained from the Arabidopsis plants transformed with SLL1282 under control of the parsley ubiquitin promoter, with and without subcellular targeting, and tested under water limited conditions.

TABLE 22

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| sll1282 | PCUbi | None | Biomass at | 27.5 | 0.0000 | 6 | 6 | 0 |
| | | | 20 days | | | | | |
| sll1282 | PCUbi | None | Biomass at | 34.5 | 0.0000 | 6 | 6 | 0 |
| | | | 27 days | | | | | |
| sll1282 | PCUbi | None | Health Index | 9.5 | 0.0003 | 6 | 6 | 0 |
| sll1282 | PCUbi | Mito | Biomass at | 8.7 | 0.0166 | 7 | 4 | 3 |
| | | | 20 days | | | | | |
| sll1282 | PCUbi | Mito | Biomass at | 10.7 | 0.0029 | 7 | 5 | 2 |
| | | | 27 days | | | | | |
| sll1282 | PCUbi | Mito | Health Index | −3.1 | NS | 7 | 3 | 4 |
| sll1282 | PCUbi | Chlor | Biomass at | −16.9 | 0.0000 | 6 | 1 | 5 |
| | | | 20 days | | | | | |
| sll1282 | PCUbi | Chlor | Biomass at | −4.6 | NS | 6 | 1 | 5 |
| | | | 27 days | | | | | |
| sll1282 | PCUbi | Chlor | Health Index | −6.8 | 0.0044 | 6 | 1 | 5 |

Arabidopsis plants that were grown under water limited conditions were significantly larger than the control plants that did not express the SLL1282 gene at two measuring times, if the protein did not contain a subcellular targeting sequence. If a mitochondrial targeting sequence was included, the gene was less effective, but did provide some improvement relative to the control. Targeting the protein to the plastid resulted in reduced growth. In these experiments, all independent transgenic events with no subcellular targeting were larger than the controls in the water limited environment.

### Q. Vitamin B6 Biosynthetic Genes

The E. coli gene designated b1636 (SEQ ID NO:79) encoding the pyridoxal kinase PdxY was expressed in Arabidopsis using a construct controlled by the Super promoter (SEQ ID NO:122) targeted to the chloroplast. Table 23 sets forth biomass and health index data obtained from Arabidopsis plants transformed with these constructs and tested under well-watered conditions.

TABLE 23

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| b1636 | super | Chlor | Biomass at | 10.7 | 0.0001 | 6 | 5 | 1 |
| | | | 17 days | | | | | |
| b1636 | super | Chlor | Biomass at | 10.0 | 0.0000 | 6 | 5 | 1 |
| | | | 21 days | | | | | |
| b1636 | super | Chlor | Health Index | −10.7 | 0.0009 | 6 | 1 | 5 |

Arabidopsis plants that were grown under well watered conditions were significantly larger than the control plants that did not express the b1636 gene at two measuring times. In these experiments, the majority of the independent transgenic events were larger than the controls in the well watered environment.

Table 24 sets forth biomass and health index data obtained from the Arabidopsis plants transformed with b1636 controlled by the super promoter targeted to the chloroplast and tested under water limited conditions.

TABLE 24

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| b1636 | super | Chlor | Biomass at | 8.9 | 0.0243 | 6 | 4 | 2 |
| | | | 20 days | | | | | |
| b1636 | super | Chlor | Biomass at | 21.9 | 0.0000 | 6 | 3 | 3 |
| | | | 27 days | | | | | |
| b1636 | super | Chlor | Health Index | 1.6 | NS | 6 | 4 | 2 |

Arabidopsis plants that were grown under water limited conditions were significantly larger than the control plants that did not express the b1636 gene at two measuring times. In these experiments, three or four of the six independent transgenic events were larger than the controls in the water limited environment.

The Synechocystis gene designated SLR1779 (SEQ ID NO:95) encoding the pyridoxal phosphate biosynthetic protein PdxJ was expressed in Arabidopsis using three different constructs controlled by the parsley ubiquitin promoter (SEQ ID NO:121): constructs with no subcellular targeting, constructs targeted to the chloroplast, and constructs targeted to mitochondria. Table 25 sets forth biomass and health index data obtained from the Arabidopsis plants transformed with these constructs and tested under well-watered conditions.

TABLE 25

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| slr1779 | PCUbi | None | Biomass at | 23.8 | 0.0000 | 8 | 8 | 0 |
| | | | 17 days | | | | | |
| slr1779 | PCUbi | None | Biomass at | 20.6 | 0.0000 | 8 | 8 | 0 |
| | | | 21 days | | | | | |
| slr1779 | PCUbi | None | Health Index | −1.1 | NS | 8 | 4 | 4 |
| slr1779 | PCUbi | Mito | Biomass at | 18.8 | 0.0000 | 6 | 6 | 0 |
| | | | 17 days | | | | | |
| slr1779 | PCUbi | Mito | Biomass at | 7.6 | 0.0008 | 6 | 6 | 0 |
| | | | 21 days | | | | | |
| slr1779 | PCUbi | Mito | Health Index | 8.7 | 0.0104 | 6 | 6 | 0 |
| slr1779 | PCUbi | Chlor | Biomass at | 27.6 | 0.0000 | 6 | 6 | 0 |
| | | | 17 days | | | | | |
| slr1779 | PCUbi | Chlor | Biomass at | 15.1 | 0.0000 | 6 | 6 | 0 |
| | | | 21 days | | | | | |
| slr1779 | PCUbi | Chlor | Health Index | −1.0 | NS | 6 | 3 | 3 |

Table 26 sets forth biomass and health index data of the Arabidopsis plants transformed with the SLR1779 gene controlled by the parsley ubiquitin promoter with subcellular targeting and tested under water limited conditions.

TABLE 26

| | | | | | | | No of | No. of |
|---|---|---|---|---|---|---|---|---|
| | | | | % | p- | No. of | Positive | Negative |
| Gene | Promoter | Targeting | Measurement | Change | Value | Events | Events | Events |
| slr1779 | PCUbi | Mito | Biomass at | −6.5 | 0.0058 | 6 | 4 | 2 |
| | | | 20 days | | | | | |
| slr1779 | PCUbi | Mito | Biomass at | −4.7 | 0.0302 | 6 | 3 | 3 |
| | | | 27 days | | | | | |
| slr1779 | PCUbi | Mito | Health Index | −0.4 | NS | 6 | 3 | 3 |
| slr1779 | PCUbi | Chlor | Biomass at | 16.6 | 0.0000 | 6 | 5 | 1 |
| | | | 20 days | | | | | |
| slr1779 | PCUbi | Chlor | Biomass at | 8.5 | 0.0002 | 6 | 4 | 2 |
| | | | 27 days | | | | | |
| slr1779 | PCUbi | Chlor | Health Index | 13.2 | 0.0000 | 6 | 5 | 1 |

Transgenic plants expressing the SLR1779 gene were larger under well watered conditions than the control plants that did not express the SLR1779 gene. This effect was observed with all three constructs that had no subcellular targeting or with subcellular targeting either to the mitochondria or to the plastid.

Transgenic plants expressing the SLR1779 gene with subcellular targeting to the plastid were significantly larger under water limited conditions than the control plants that did not express the SLR1779 gene. In addition, the transgenic plants were darker green in color than the controls under water limited conditions as shown by the increased health index. In these experiments, the majority of the independent transgenic events were larger than the controls in the water limited environment.

Transgenic plants expressing the SLR1779 gene with subcellular targeting to the mitochondria were significantly smaller under water limited conditions than the control plants that did not express the SLR1779 gene.

1-8. (canceled)

9. A method of producing a transgenic plant having enhanced yield as compared to a wild type plant of the same variety, the method comprising the steps of
a) transforming a plant cell with an expression vector comprising, in operative association,
i) an isolated polynucleotide encoding a promoter;
ii) an isolated polynucleotide encoding a subcellular targeting peptide; and
iii) an isolated polynucleotide encoding a polypeptide comprising a CTP_transf_{—}1 signature sequence selected from the group consisting of amino acids 65 to 377 of SEQ ID NO:2, amino acids 54 to 367 of SEQ ID NO:4, and amino acids 53 to 366 of SEQ ID NO:6;
b) regenerating transgenic plants from the transformed plant cell; and
c) selecting higher-yielding plants from the regenerated transgenic plants.

10. (canceled)

11. The method of claim 9, wherein the polypeptide comprises amino acids 1 to 457 of SEQ ID NO: 2, amino acids 1 to 367 of SEQ ID NO: 4, or amino acids 1 to 425 of SEQ ID NO: 6.

12. (canceled)

13. A method of producing a transgenic plant having enhanced yield as compared to a wild type plant of the same variety, the method comprising the steps of:
a) transforming a plant cell with an expression vector comprising, in operative association,
i) an isolated polynucleotide encoding a promoter capable of enhancing expression in leaves;
ii) an isolated polynucleotide encoding a mitochondrial transit peptide; and
iii) an isolated polynucleotide encoding a polypeptide comprising a phosphopantetheine binding domain having a sequence selected from the group consisting of amino acids 77 to 92 of SEQ ID NO:8; amino acids 73 to 88 of SEQ ID NO: 10; amino acids 75 to 90 of SEQ ID NO: 12; and amino acids 84 to 99 of SEQ ID NO: 14;
b) regenerating transgenic plants from the transformed plant cell; and
c) selecting higher-yielding plants from the regenerated transgenic plants.

14. The method of claim 13, wherein the polypeptide has a sequence comprising amino acids 1 to 125 of SEQ ID NO:8; amino acids 1 to 117 of SEQ ID NO:10; amino acids 1 to 127 of SEQ ID NO:12; or amino acids 1 to 119 of SEQ ID NO:14.

15. A method of producing a transgenic plant having enhanced yield as compared to a wild type plant of the same variety, the method comprising the steps of:
a) transforming a plant cell with an expression vector comprising, in operative association,
i) an isolated polynucleotide encoding a promoter;
ii) an isolated polynucleotide encoding a subcellular targeting peptide; and
iii) an isolated polynucleotide encoding a polypeptide comprising an acyltransferase domain selected from the group consisting of amino acids 108 to 272 of SEQ ID NO:16; amino acids 80 to 222 SEQ ID NO:18; and amino acids 116 to 258 SEQ ID NO:208;
b) regenerating transgenic plants from the transformed plant cell; and
c) selecting higher-yielding plants from the regenerated transgenic plants. 16 (new). The method of claim 15, wherein the polypeptide comprises amino acids 1 to 396 of SEQ ID NO:16, amino acids 1 to 384 of SEQ ID NO:18, or amino acids 1 to 332 of SEQ ID NO:20.

17. A method of producing a transgenic plant having enhanced yield as compared to a wild type plant of the same variety, the method comprising the steps of:
a) transforming a plant cell with an expression vector comprising, in operative association,
i) an isolated polynucleotide encoding a promoter capable of enhancing expression in leaves;
ii) an isolated polynucleotide encoding a mitochondrial transit peptide; and
iii) an isolated polynucleotide encoding a polypeptide comprising:
A) an ECH domain selected from the group consisting of amino acids 17 to 190 of SEQ ID NO:22; amino acids 17 to 187 of SEQ ID NO:24, and amino acids 18 to 187 of SEQ ID NO:26;
B) an 3HCDH domain selected from the group consisting of amino acids 489 to 513 of SEQ ID NO:22; amino acids 490 to 514 of SEQ ID NO:24, and amino acids 490 to 514 of SEQ ID NO:26; and
C) an 3HCDH-N domain selected from the group consisting of amino acids 310 to 490 of SEQ ID NO:22; amino acids 312 to 491 of SEQ ID NO: 24, and amino acids 312 to 490 of SEQ ID NO:26;
b) regenerating transgenic plants from the transformed plant cell; and
c) selecting higher-yielding plants from the regenerated transgenic plants.

18. The method of claim 17, wherein the polypeptide comprises amino acids 1 to 714 of SEQ ID NO:22, amino acids 1 to 723 of SEQ ID NO:24, or amino acids 1 to 727 of SEQ ID NO:26.

19. A transgenic plant transformed with an expression cassette comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a polypeptide selected from the group consisting of:
i) a polypeptide comprising a domain selected from the group consisting of amino acids 54 to 138 of SEQ ID NO:30, amino acids 127 to 211 of SEQ ID NO:32, and amino acids 3 to 87 of SEQ ID NO:34; and
ii) a polypeptide comprising a domain having amino acids 54 to 227 of SEQ ID NO:38.

20. The transgenic plant of claim 19, wherein the polypeptide comprises amino acids 1 to 248 of SEQ ID NO:30, amino acids 1 to 212 of SEQ ID NO:32, amino acids 1 to 197 of SEQ ID NO:34, or amino acids 1 to 264 of SEQ ID NO:38.

21. A transgenic plant transformed with an expression cassette comprising, in operative association:
a) an isolated polynucleotide encoding a promoter; and
b) an isolated polynucleotide selected from the group consisting of:
i) a polynucleotide encoding a polypeptide comprising amino acids 1 to 401 of SEQ ID NO:42;
ii) a polynucleotide encoding a polypeptide comprising amino acids 1 to 261 of SEQ ID NO:44; and
iii) a polynucleotide encoding a polypeptide comprising amino acids 50 to 544 of SEQ ID NO:70.

22. A transgenic plant transformed with an expression cassette comprising, in operative association,
a) an isolated polynucleotide encoding a promoter capable of enhancing expression in leaves, and
b) an isolated polynucleotide encoding a polypeptide comprising a domain selected from the group consisting of amino acids 101 to 356 of SEQ ID NO:46; amino acids 97 to 353 of SEQ ID NO:48; amino acids 91 to 346 of SEQ ID NO:50; and amino acids 100 to 355 of SEQ ID NO:52.

23. The transgenic plant of claim 22, wherein the polypeptide comprises amino acids Ito 393 of SEQ ID NO:46, amino acids 1 to 368 of SEQ ID NO:48; amino acids 1 to 363 of SEQ ID NO:50, or amino acids 1 to 379 of SEQ ID NO:52.

24. A transgenic plant transformed with an expression cassette comprising, in operative association,
a) an isolated polynucleotide encoding a promoter capable of enhancing gene expression in roots and shoots and
b) an isolated polynucleotide encoding a polypeptide selected from the group consisting of
i) a polypeptide comprising a DJ-1_Pfp1 signature sequence selected from the group consisting of amino acids 46 to 208 of SEQ ID NO:54; amino acids 32 to 189 of SEQ ID NO:56; and amino acids 25 to 151 of SEQ ID NO:58;
ii) a polypeptide comprising amino acids 14 to 195 of SEQ ID NO:60;
iii) a polypeptide comprising amino acids 1 to 102 of SEQ ID NO:64; and iv) a polypeptide comprising a first YscJ/FliF signature sequence comprising amino acids 17 to 225 of SEQ ID NO:66 and a second YscJ/FliF signature sequence comprising amino acids 250 to 429 of SEQ ID NO:66.

25. The transgenic plant of claim 24, wherein the polypeptide comprises amino acids 1 to 220 of SEQ ID NO:54, amino acids 1 to 231 of SEQ ID NO:56, amino acids 1 to 161 of SEQ ID NO:58, amino acids 1 to 195 of SEQ ID NO:60, or amino acids 1 to 552 of SEQ ID NO:66.

26. A transgenic plant transformed with an expression cassette comprising, in operative association,
a) an isolated polynucleotide encoding a promoter and
b) an isolated polynucleotide encoding a lumazine synthase polypeptide which does not comprise a plastid targeting sequence, wherein the polypeptide comprises a DMRL_synthase signature sequence selected from the group consisting of amino acids 12 to 158 of SEQ ID NO:72; amino acids 83 to 226 of SEQ ID NO:74; amino acids 70 to 213 of SEQ ID NO:76, amino acids 70 to 215 of SEQ ID NO:78.

27. The transgenic plant of claim 26, wherein the polypeptide comprises amino acids 1 to 164 of SEQ ID NO:72, amino acids 54 to 228 of SEQ ID NO:74, amino acids 57 to 217 of SEQ ID NO:76, or amino acids 81 to 217 of SEQ ID NO:78.

28. A transgenic plant transformed with an expression cassette comprising, in operative association,
a) an isolated polynucleotide encoding a promoter; and
b) an isolated polynucleotide encoding a full length polypeptide which is capable of enhancing pyridoxal 5′-phosphate synthesis.

29. The transgenic plant of claim 28, wherein the polypeptide is selected from the group consisting of SEQ ID NO:80, SEQ ID NO:82; SEQ ID NO:84; SEQ ID NO:86; SEQ ID NO:88; SEQ ID NO:90; SEQ ID NO:92; SEQ ID NO:94; SEQ ID NO:96; SEQ ID NO:98; SEQ ID NO:100; SEQ ID NO:102; SEQ ID NO:104; SEQ ID NO:106; SEQ ID NO:108; SEQ ID NO:110; SEQ ID NO:112; SEQ ID NO:114; SEQ ID NO:116; SEQ ID NO:118; and SEQ ID NO:120.

30. An isolated polynucleotide encoding a polypeptide having an amino acid sequence selected from the group consisting of SEQ ID NO:4; SEQ ID NO:6; SEQ ID NO:10; SEQ ID NO:12; SEQ ID NO:14; SEQ ID NO:18; SEQ ID NO:20; SEQ ID NO:24; SEQ ID NO:26; SEQ ID NO:30; SEQ ID NO:32; SEQ ID NO:34; SEQ ID NO:38; SEQ ID NO:48; SEQ ID NO:50; SEQ ID NO:52; SEQ ID NO:56; SEQ ID NO:58; SEQ ID NO:70; SEQ ID NO:74; SEQ ID NO:76; and SEQ ID NO:78.
